(19) United States
(12) Patent Application Publication
(10) Pub. No.: US 2009/0209578 A1
(43) Pub. Date: Aug. 20, 2009

(54) CHEMICAL COMPOUNDS

(75) Inventors: Christopher Gordon Barber, Kent (GB); David Clive Blakemore, Kent (GB)

Correspondence Address: PFIZER INC;Steve T. Zelson, 150 EAST 42ND STREET, 5TH FLOOR - STOP 49, NEW YORK, NY, 10017-5612, US

(73) Assignee: PFIZER INC

(21) Appl. No.: 12/096,406

(22) PCT Filed: Nov. 27, 2006
(86) PCT No.: PCT/IB2006/003480

§ 371 Date: Dec. 12, 2008

Related U.S. Application Data
(60) Provisional application No. 60/748,867, filed on Dec. 8, 2005.

(51) Int. Cl. A61K 31/454; C07D 471/04; C07D 413/04; C07D 401/04; A61K 31/437; A61K 31/4545; A61P 31/12; A61P 31/18

(52) U.S. Cl. 514/303; 546/118; 546/209; 546/210; 546/211; 514/326; 514/316

(57) ABSTRACT

The present invention provides compounds of formula (I) wherein R¹, R², R³, R⁴, Het and m are as defined in the description. The compounds of the present invention are modulators, especially antagonists, of the activity of chemokine CCR5 receptors.

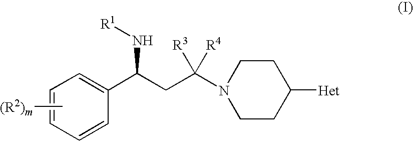

This invention relates to piperidine derivatives, to processes for their preparation, to compositions containing them and to their use.

More particularly, the present invention relates to the use of alpha-methyl piperidine derivatives in the treatment of a variety of disorders, including those in which the modulation of chemokine CCR5 receptors is implicated. Accordingly, the compounds of formula (I) are in particular useful in the treatment of HIV, such as HIV-1, and genetically related retroviral infections (and the resulting acquired immune deficiency syndrome, AIDS), inflammatory diseases, autoimmune diseases and pain.

The name “chemokine”, is a contraction of “chemotactic cytokines”. The chemokines comprise a large family of proteins which have in common important structural features and which have the ability to attract leukocytes. As leukocyte chemotactic factors, chemokines play an indispensable role in the attraction of leukocytes to various tissues of the body, a process which is essential for both inflammation and the body's response to infection. Because chemokines and their receptors are central to the pathophysiology of inflammatory and infectious diseases, agents which are active in modulating, preferably antagonizing, the activity of chemokines and their receptors, are useful in the therapeutic treatment of such inflammatory and infectious diseases.

The chemokine receptor CCR5 is of particular importance in the context of treating inflammatory and infectious diseases. CCR5 is a receptor for chemokines, especially for the macrophage inflammatory proteins (MIP) designated MIP-1α and MIP-1β, and for a protein which is regulated upon activation and is normal T-cell expressed and secreted (RANTES).

Acquired Immune Deficiency Syndrome (AIDS) causes a gradual breakdown of the body's immune system as well as progressive deterioration of the central and peripheral nervous systems. Since its initial recognition in the early 1980's, AIDS has spread rapidly and has now reached epidemic proportions within a relatively limited segment of the population. Intensive research has led to the discovery of the responsible agent, human T-lymphotropic retrovirus III (HTLV-III), now more commonly referred to as the human immunodeficiency virus or HIV.

HIV is a member of the class of viruses known as retroviruses. The retroviral genome is composed of RNA which is converted to DNA by reverse transcription. This retroviral DNA is then stably integrated into a host cell's chromosome and, employing the replicative processes of the host cells, produces new retroviral particles and advances the infection to other cells. HIV appears to have a particular affinity for the human T-4 lymphocyte cell which plays a vital role in the body's immune system. HIV infection of these white blood cells depletes this white cell population. Eventually, the immune system is rendered inoperative and ineffective against various opportunistic diseases such as, among others, pneumocystic carini pneumonia, Kaposi's sarcoma, and cancer of the lymph system.

Although the exact mechanism of the formation and working of the HIV virus is not understood, identification of the virus has led to some progress in controlling the disease. For example, the drug azidothymidine (AZT) has been found effective for inhibiting the reverse transcription of the retroviral genome of the HIV virus, thus giving a measure of control, though not a cure, for patients afflicted with AIDS. The search continues for drugs that can cure or at least provide an improved measure of control of the deadly HIV virus.

CCR5 antagonists for the treatment of HIV infections have been described in, for example, WO 00/39125, EP 1 013 276, WO 03/084954 and WO 05/033107. CCR5 antagonists are described in co-pending (but unpublished) PCT patent application no. PCT/IB2006/001669.

It is desirable to provide compounds for treatment of HIV and other indications which have one or more of the following properties: are selective, have a rapid onset of action, are potent, are stable, are resistant to metabolism, or have other desirable drug-like properties.

According to one aspect of the invention there is provided a compound of formula (I):

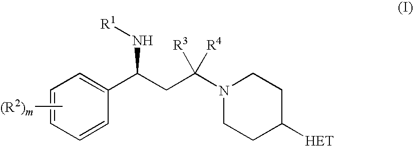

or a pharmaceutically acceptable salt, solvate or derivative thereof, wherein:

### R¹ is COR⁵, CO₂R⁵, CONR⁶R⁷;

R² is halogen, cyano, CF₃, C₁₋₄alkyl or C₁₋₄alkyloxy;
R³ is C₁₋₄ alkyl;
R⁴ is H or C₁₋₄alkyl;
R⁵ is C₁₋₆alkyl, C₃₋₇cycloalkyl or C₃₋₇cycloalkylC₁₋₂alkyl, wherein said alkyl and cycloalkyl are substituted by 0 to 3 halogen atoms; or a 4 to 7-membered saturated heterocycle containing one 0 or one S atom, wherein said S atom is substituted by 0 to 2 oxo groups;
R⁶ is C₁₋₆alkyl;
R⁷ is H or C₁₋₆alkyl;
m is 0, 1 or 2; and

HET is a
(i) a 5 membered monocylic aromatic heterocycle containing from 1 to 4 heteroatoms selected from O, S and N, which is substituted by 0 to 3 groups independently selected from C₁₋₄ alkyl, C₃₋₆cycloalkyl,
C₁₋₄alkyloxy or C₁₋₄alkyloxyC₁₋₄alkyl; or
(ii) a tetrahyrodroimadazopyridine of formula

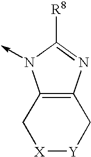

wherein:
R⁸ is methyl or ethyl substituted by 0 to 3 fluorine atoms;
X and Y are selected from CH₂ and NR⁹ such that one of X and Y is CH₂ and the other is NR⁹; and
R⁹ is COR⁶, CO₂R⁶ or CONR⁶R⁷
   With the proviso that:
   (i) R⁵ is not a tertiary alkyl group; and
   (ii) HET is not a 1,2,4-triazole or a 1,3,4-triazole.

The term “alkyl” refers to a straight-chain or branched-chain saturated aliphatic hydrocarbon radical containing the specified number of carbon atoms. Examples of alkyl radicals include, but are not limited to, methyl, ethyl, n-propyl, isopropyl, n-butyl, isobutyl, sec-butyl, tert-butyl, n-pentyl, isoamyl, n-hexyl.

The term “alkyloxy” refers to a group —OR in which R is an alkyl as defined above.

The term “cycloalkyl” refers to a carbocyclic ring containing the specified number of carbon atoms. Examples of carbocyclic rings include cyclopropyl, cyclobutyl, cyclopentyl, cyclohexyl and cycloheptyl.

The term “halogen” refers to fluorine, chlorine, bromine or iodine.

When a heterocycle contains one or more nitrogen atoms, N-oxides are included within the scope of the invention.

In one embodiment of the invention, HET is a 5 membered monocylic aromatic heterocycle containing from 1 to 4 heteroatoms selected from O, S and N, which is substituted by 0 to 3 groups independently selected from C₁₋₄ alkyl, C₃₋₆cycloalkyl, C₁₋₄alkyloxy or C₁₋₄alkyloxyC₁₋₄alkyl.

In another embodiment, HET in formula (I) is selected from the following moieties:

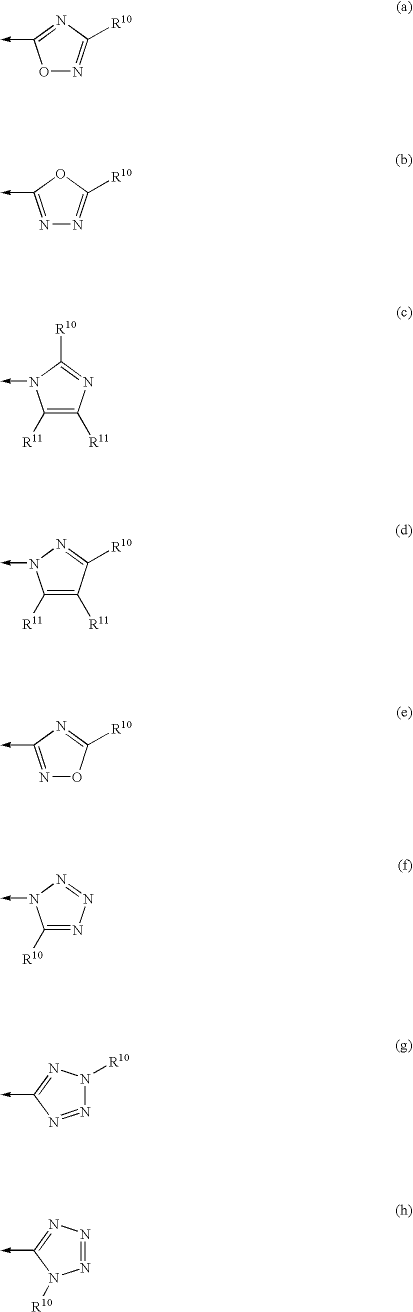

wherein R¹⁰ and each R¹¹ are independently selected from H, C₁₋₄ alkyl, C₃₋₆cycloalkyl, C₁₋₄alkyloxy and C₁₋₄alkyloxyC₁₋₄alkyl.

It will be appreciated that each of HET structures (a) to (h) above form yet further, separate, embodiments of the invention.

In another embodiment, R¹⁰ is C₁₋₄ alkyl, C₃₋₆cycloalkyl, C₁₋₄alkyloxy or C₁₋₄alkyloxy-C₁₋₄alkyl; and R¹¹ is H.

In another embodiment, R¹⁰ is C₁₋₄ alkyl.

In another embodiment, HET is a tetrahydroimadazopyridine to define compounds of formula (1A)

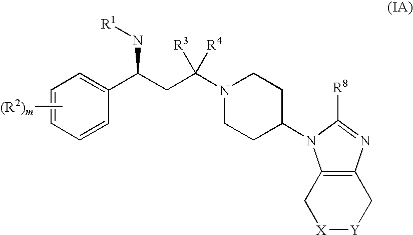

wherein R¹ to R⁴ are as defined above, R⁸ is methyl or ethyl substituted by 0 to 3 fluorine atoms; one of X and Y are selected from CH₂ and NR⁹ such that one of X and Y is CH₂ and the other is NR⁹; and
R⁹ is COR⁶, CO₂R⁶ or CONR⁶R⁷, wherein R⁶ and R⁷ are as defined above.
In one embodiment, R⁸ is methyl.

In another embodiment, HET is a tetrahydroimadazopyridine of formula

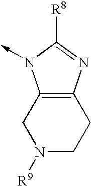

wherein R⁸ and R⁹ is as defined in the first aspect of the invention.

In another embodiment, HET is a tetrahyrdoimazopyridine of formula

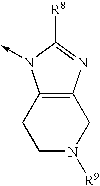

wherein R⁸ and R⁹ are as defined in the first aspect of the invention.

In another embodiment, R⁹ is COR⁶ or CO₂R⁶.

In another embodiment, R⁶ is methyl, ethyl, n-propyl or isopropyl.

In another embodiment, R⁵ is 1,1-dioxo-tetrahydrothiopyran or tetrahydropyran.

In another embodiment, R¹ is COR⁵ or CO₂R⁵.

In another embodiment, R¹ is COR⁵ or CO₂R⁵ wherein R⁵ is C₁₋₄alkyl or C₃₋₇cycloalkyl and wherein the cycloalkyl is optionally substituted by 0 to 2 fluoro atoms.

In another embodiment, R₂ is halogen and in another embodiment it is fluorine.

In another embodiment, m is 0 or 1.

In another embodiment, m is 0.

In another embodiment, m is 1.

In another embodiment, R³ is methyl.

In another embodiment, R⁴ is H.

In another embodiment, R¹ is COR⁵ or CO₂R⁵ wherein R⁵ is C₁₋₄alkyl; m is 0 or 1; R² is halogen; R³ is methyl; R⁴ is H; R⁸ is methyl; and R⁹ is COR⁶ or CO₂R⁶, wherein R⁶ is C₁₋₄alkyl.

The compounds of the following examples are within the scope of formula (I): 5, 6, 7, 8, 9A, 9B, 10, 11, 12, 13A, 13B, 14, 21, 22 to 31, 47 to 69, 72, 73, 76 to 80, 91 to 105, 109 to 112, 116, 117, 121, 122 and 125 to 131. The compounds of these examples have an IC50 in the cell fusion assay (described later) of less than 1.5 micro Molar.

The remaining examples (many of which also have activity in the cell fusion assay) have utility in the preparation of the above examples falling within formula (I) and form a further aspect of the invention.

It will be appreciated that novel intermediates herein which are used in the preparation of a compound of formula (I) form yet a further aspect of the invention.

The compounds of the following examples have activity of less than 5 nM in our cell fusion assay: compounds of examples 5, 6, 7, 9A, 10, 11, 12, 21 to 25, 47 to 58, 60 to 69, 72, 77 to 80, 92, 94, 96, 99, 100, 103, 104, 109, 111, 112, 121 and 127.

Most preferred are the following compounds of examples 56, 58, 62, 66, 92, 94 and 104, that is:
N-{(1S)-3-[4-(5-acetyl-2-methyl-4,5,6,7-tetrahydro-3H-imidazo[4,5-c]pyridin-3-yl)piperidin-1-yl]-1-phenylbutyl}butanamide;
N-[(1S)-3-[4-(5-acetyl-2-methyl-4,5,6,7-tetrahydro-3H-imidazo[4,5-c]pyridin-3-yl)piperidin-1-yl]-1-(3-fluorophenyl)butyl]-2-methylpropanamide;
N-[(1S)-3-[4-(5-acetyl-2-methyl-4,5,6,7-tetrahydro-3H-imidazo[4,5-c]pyridin-3-yl)piperidin-1-yl]-1-(3-fluorophenyl)butyl]propanamide;
ethyl 3-{1-[(3S)-3-(acetylamino)-3-(3-fluorophenyl)-1-methylpropyl]piperidin-4-yl}-2-methyl-3,4,6,7-tetrahydro-5H-imidazo[4,5-c]pyridine-5-carboxylate;
methyl 2-methyl-1-{1-[(3S)-1-methyl-3-phenyl-3-(propionylamino)propyl]piperidin-4-yl}-1,4,6,7-tetrahydro-5H-imidazo[4,5-c]pyridine-5-carboxylate; N-[(1S)-3-[4-(5-acetyl-2-methyl-4,5,6,7-tetrahydro-1H-imidazo[4,5-c]pyridin-1-yl)piperidin-1-yl]-1-(3-fluorophenyl)butyl]-2-methylpropanamide; and
N-[(1S)-3-[4-(5-acetyl-2-methyl-4,5,6,7-tetrahydro-1H-imidazo[4,5-c]pyridin-1-yl)piperidin-1-yl]-1-(3-fluorophenyl)butyl]propanamide;
   or a pharmaceutically acceptable salts, solvates or derivatives thereof.

It is to be understood that the invention covers all combinations of particular embodiments of the invention as described hereinabove, consistent with the definition of the compounds of formula (I).

The invention includes the compounds of formula (I) and pharmaceutically acceptable salts, solvates or derivatives thereof (wherein derivatives include complexes, prodrugs, polymorphs and crystal habits thereof, and isotopes, as well as salts and solvates thereof) and reference to compounds of formula (I) should be construed accordingly.

Pharmaceutically acceptable salts of the compounds of formula (I) include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002), incorporated herein by reference.

The compounds of formula (I) may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of formula (I) may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of formula (I) may also exist in multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The compounds of formula (I) may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970), incorporated herein by reference.

As indicated, so-called ‘prodrugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association), both incorporated herein by reference.

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985), incorporated herein by reference.

Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include:

(i) where the compound of formula (I) contains a methyl group, an hydroxymethyl derivative thereof (—CH₃->-CH₂OH);
(ii) where the compound of formula (I) contains an alkoxy group, an hydroxy derivative thereof (—OR->-OH);
(iii) where the compound of formula (I) contains a tertiary amino group, a secondary amino derivative thereof (—NR¹R²—>—NHR¹ or —NHR²);
(iv) where the compound of formula (I) contains a secondary amino group, a primary derivative thereof (—NHR¹—>—NH₂);
(v) where the compound of formula (I) contains a phenyl moiety, a phenol derivative thereof (-Ph->-PhOH); and
(vi) where the compound of formula (I) contains an amide group, a carboxylic acid derivative thereof (—CONH₂->COOH).

The compounds of formula (I) contain one or more asymmetric carbon atoms, which are depicted in formula (I) below by an asterisk.

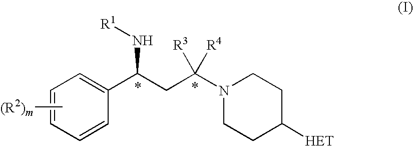

The bonds from an asymmetric carbon in compounds of the present invention may be depicted herein using a solid line (—), a zigzag line ( [symbol] ), a solid wedge ( [symbol] ), or a dotted wedge ( [symbol] ). The use of either a solid or dotted wedge to depict bonds from an asymmetric carbon atoms is meant to indicate that only the stereoisomer shown is meant to be included.

The compounds of formula (I) also contain more than one asymmetric carbon atom. In those compounds, the use of a solid line to depict bonds from asymmetric carbon atoms is meant to indicate that all possible stereoisomers are meant to be included, unless it is clear from the context that a specific stereoisomer is intended. In particular, in the following examples a single stereoisomer is formed, but its absolute configuration is not defined: examples 2, 3, 15 to 69, 72, 73, 81 to 84, 87 to 104, 106A, 106B, 107 to 110, 113A, 113B, 114 to 117, 118A, 118B, 119 to 122, 125, 126, 132, to 134. In these examples the bonds from the asymmetric carbon atoms are indicated by the use of a solid wedge and a solid line and indicate that a single stereoisomer of undefined absolute configuration is present.

The use of a zigzag line to depict bonds from one or more asymmetric carbon atoms in a compound of the invention and the use of a solid or dotted wedge to depict bonds from other asymmetric carbon atoms in the same compound is meant to indicate that a mixture of diastereomers is present.

Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism.

Consistent with the definition of formula (I) included within the scope of the present invention are all stereoisomers, diastereomers, geometric isomers and tautomeric forms of the compounds of formula (I), including compounds exhibiting more than one type of isomerism, and mixtures of one or more thereof. Also included are acid addition or base salts wherein the counterion is optically active, for example, d-lactate or 1-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

The present invention also includes all pharmaceutically acceptable isotopically-labelled compounds of formula (I) wherein one or more atoms are replaced by atoms having the same atomic number, but an atomic mass or mass number different from the atomic mass or mass number which predominates in nature.

In the general processes, and schemes, that follow: AcOH is acetic acid; DCC is N,N′-dicyclohexylcarbodiimide; DCM is dichloromethane; DIPEA is diisopropylethylamine; DMF is N,N-dimethylformamide; EDCl is 1-ethyl-3-(3-dimethylaminopropyl)carbodiimide; Et₃N is triethylamine; EtOAc is ethyl acetate; EtOH is ethanol; HATU is O-(7-Azabenzotriazol-1-yl)-N,N,N′,N′-tetramethyluronium hexafluorophosphate; HBTU is O-Benzotriazol-1-yl-N,N,N′,N′-tetramethyluronium-hexafluorophosphate; HCl is hydrogen chloride; HOBT is 1-Hydroxybenzotriazole; MeI is methyl iodide; MeOH is methanol; THF is tetrahydrofuran; rt is room temperature.

The compounds of formula (I) may be prepared by any process used for preparing analogous compounds.

All R variatiables, HET and m are as defined above and PG¹ represents a suitable N-protecting group known to those skilled in the art.

It will be appreciated by those skilled in the art that, as illustrated in the schemes that follow, it may be necessary or desirable at any stage in the synthesis of compounds of formula (I) to protect one or more sensitive groups in the molecule so as to prevent undesirable side reactions. In particular, it may be necessary or desirable to protect amino groups. The protecting groups used in the preparation of compounds of formula (I) may be used in conventional manner. See, for example, those described in ‘Protective Groups in Organic Synthesis’ by Theodora W Green and Peter G M Wuts, third edition, (John Wiley and Sons, 1999), in particular chapter 7, pages 494-653 (“Protection for the Amino Group”), incorporated herein by reference, which also describes methods for the removal of such groups.

The amino protecting groups t-butoxycarbonyl (Boc), 9-fluorenylmethoxycarbonyl (Fmoc), benzyloxycarbonyl (Cbz), methylformate, benzyl and acetyl are of particular use in the preparation of compounds of formula (I) and intermediates thereto.

The compounds of formula (V) may be prepared as depicted in Scheme 1

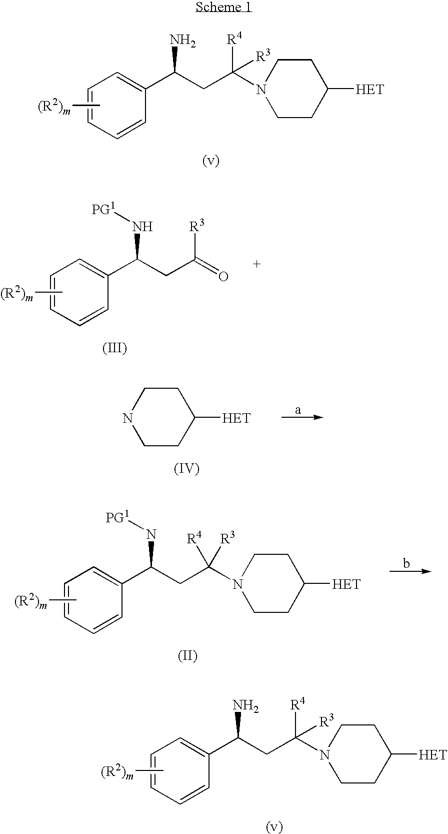

a. Reductive amination is carried out using the standard literature conditions, preferably in the presence of titanium tetraisopropoxide using a reducing agent (preferably sodium cyanoborohydride or sodium triacetoxyborohydride) in a suitable solvent such as ethanol or dichloromethane/methanol at room temperature. Separation of diastereoisomers can be carried out at this stage by, for example, flash chromatography on silica.
b. Deprotection of the amine is carried out as follows:
   Removal of a Boc group is carried out under acidic conditions e.g. hydrogen chloride dissolved in ether/methanol or ethyl acetate solution at 0° C. to room temperature or trifluoroacetic acid in dichloromethane.
   Removal of a CBz group is carried out by hydrogenolysis e.g. using a suitable catalyst (e.g. palladium hydroxide or palladium on carbon) under a hydrogen atmosphere and in a suitable solvent such as methanol or ethanol or using ammonium formate and palladium hydroxide in a suitable solvent, such as methanol or ethanol, under reflux. Scheme 1 step (a) above illustrates the preparation of compounds of formula (II) where R⁴ is H. Where R⁴ is C₁₋₄ alkyl in formula (II), the following alternative conditions may be used.

The following is alternative conditions for the preparation of compounds of Formula (I) wherein R⁴ is C₁₋₄ alkyl.

The same precursors amine and ketone respectively of formulae (III) and (IV) are used but the imine is formed but reacted with —CN. The resultant cyanoamine is then reacted with an organometallic reagent to displace the CN with the R⁴ of the organometallic reagent. More particularly the cyanoamine is formed using a source of cyanide such as diethylaluminium cyanide in the presence of a suitable Lewis acid such as titanium tetraisopropoxide in a suitable solvent such as dichloromethane at room temperature. The cyanoamine is then converted to the dialkylamine using an alkylorganometallic (e.g. Grignard reagent) such as alkylmagnesium chloride or bromide or alkylithium in a suitable solvent such as diethyl ether or tetrahydrofuran as a suitable temperature around zero degrees centigrade.

Compounds of formula (I) may be prepared according to scheme 2.

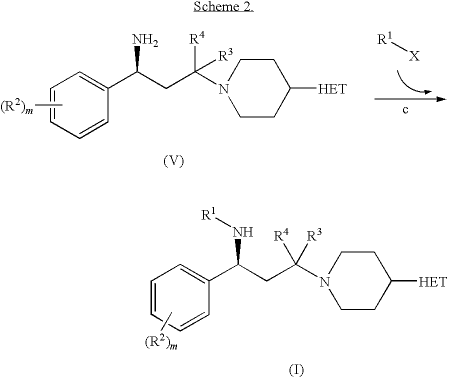

X represents a leaving group such as Cl or Br or a group (such as OH) capable of being converted to a leaving group in the presence of a suitable coupling agent,
c. When X═Cl, amine (V) is reacted with R¹Cl such as the acid chloride R⁵COCl or chloroformate in the presence of a base such as triethylamine or diisopropylethylamine in a suitable solvent such as dichloromethane or toluene at a temperature between 0° C. and room temperature.
   When X═OH, amine (V) is reacted with R¹OH such as the acid R⁵CO₂H in the presence of a coupling agent such as EDCl.HCl, HBTU, HATU, DCC or preferentially EDCl.MeI in a suitable solvent such as dichloromethane or DMF. In the presence of EDCl.HCl or EDCl.MeI, HOBT is optionally added. In the case where the amine is present as a hydrochloride salt, a suitable base such as triethylamine or diisopropylethylamine is added. The reaction is typically carried out at room temperature.

Compounds of formula (I) may alternatively be prepared as shown in scheme 3.

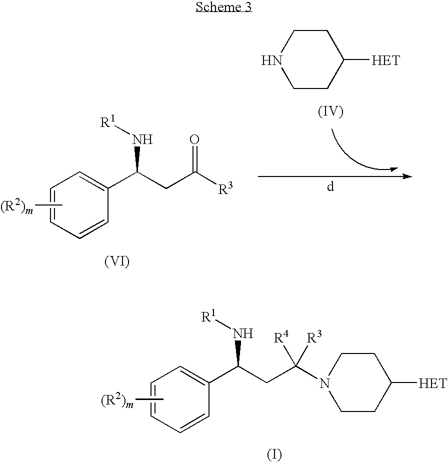

d. Reductive amination may be carried out in the presence of titanium tetraisopropoxide using a reducing agent (preferably sodium cyanoborohydride or alternatively sodium triacetoxyborohydride) in a suitable solvent such as ethanol or dichloromethane/methanol at room temperature. Separation of diastereoisomers can be carried out at this stage by, for example, flash chromatography on silica.

In scheme 3 step (d) above R⁴ is hydrogen. For formation of formula (I) where R⁴ is alkyl, the ketone and amine precursors formulae (VI) and (IV) respectively are reacted under the “alternative conditions” referenced in scheme 1.

Various piperidine HETs according to general formula (IV) may be prepared according to schemes 4 to 10 below.

A piperidine HET of formula (XIII) can be formed as follows in scheme 4.

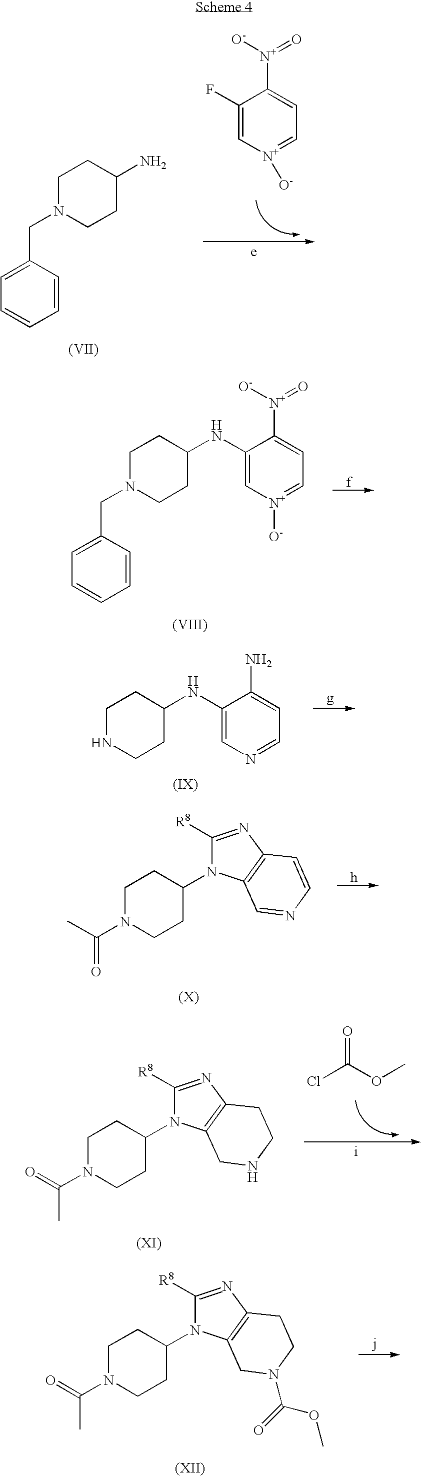

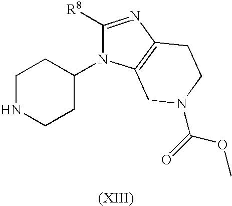

e. Amine (VII) is reacted with 3-Fluoro-4-nitropyridine 1-oxide in a solvent such as acetonitrile at a temperature typically between 0° C. and room temperature to give (VIII).
f. Hydrogenation of (VIII) is typically carried out in a suitable solvent system such as ethanol/water/acetic acid in the presence of a suitable catalyst such as Degussa E101 5% palladium on carbon under a hydrogen atmosphere at a suitable temperature typically around 40° C.
g. Ring closure is carried out by reacting amine (IX) with an acetylating agent to introduce R⁴, (such as acetic anhydride to introduce methyl) at elevated temperature typically around 100° C.
h. Hydrogenation of (X) is carried out using a suitable catalyst such as platinum oxide in an acidic solvent system such as ethanol/5N hydrochloric acid under a hydrogen atmosphere (typically around 50 Psi) at a temperature of around 50° C.
i. Methyl carbamate formation is carried out by reacting (XI) with methyl chloroformate and a suitable base such as triethylamine or diisopropylethylamine in a suitable solvent such as dichloromethane at a temperature typically around −10° C. to room temperature.
j. The amide protecting group was removed under acidic conditions preferably by reaction with diluted hydrochloric acid (typically around 2N) at elevated temperature (preferably around 65° C.).

A piperidine HET of formula (XXIII) can be formed as follows in schemes 5 and 6.

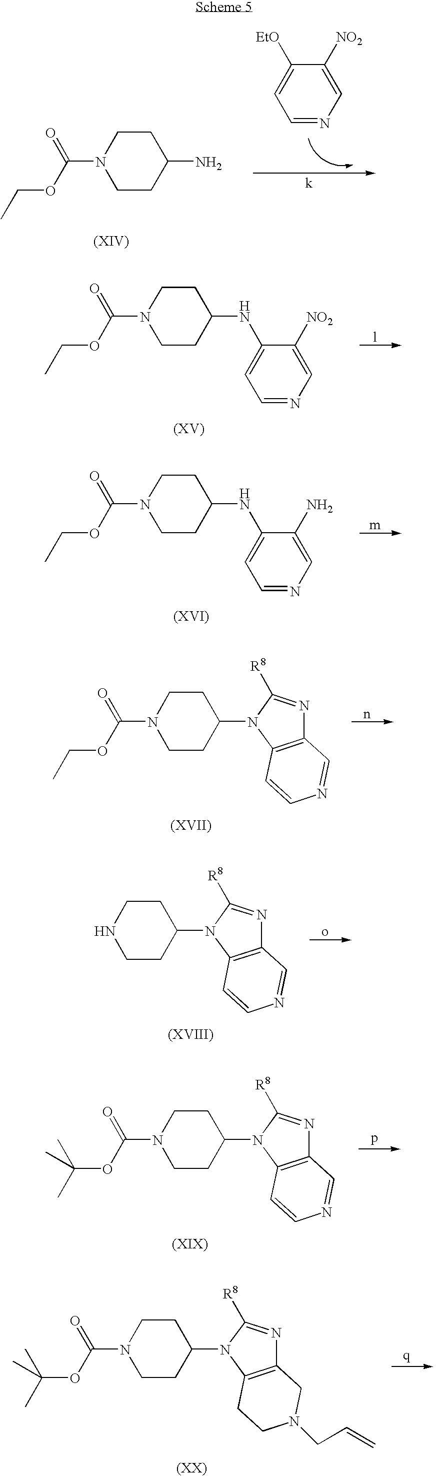

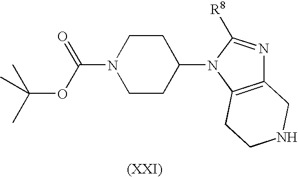

k. (XIV) is reacted with 4-ethoxy-3-nitropyridine hydrochloride in the presence of a suitable base such as triethylamine or diisopropylethylamine in a suitable solvent such as acetonitrile at elevated temperature (preferably around 90° C.).
l. Reduction of the nitro group of (XV) is typically carried out in a suitable solvent system such as methanol or ethanol in the presence of a suitable catalyst such as palladium on carbon under a hydrogen atmosphere at a suitable temperature typically around room temperature.
m. Ring closure is carried out by reacting amine (XVI) with an acetylating agent such as acetic anhydride at elevated temperature typically around 100° C. to reflux.
n. Removal of the ethyl carbamate protecting group is carried out under basic conditions preferably by refluxing with an ethanolic solution of sodium or potassium hydroxide.
o. BOC protection of (XVIII) is carried out under standard literature conditions such as by reaction with di-t-butyl dicarbonate with or without the presence of a suitable base such as triethylamine or diisopropylethylamine in a solvent such as dichloromethane typically at room temperature.
p. Alkylation of the pyridyl nitrogen is carried out in a suitable solvent such as ethanol with allyl bromide and sodium iodide at elevated temperature (preferably reflux) and subsequent reduction of the ring is carried out in the presence of a suitable reducing agent such as sodium borohydride at room temperature.
q. Removal of the allyl groups is carried out using N,N′-dimethylbarbituric acid and tetrakis(triphenylphosphine)palladium in a solvent such as dichloromethane at room temperature to reflux or using an acid such as methanesulphonic acid and palladium on carbon in a solvent such as water at reflux or using tris(triphenylphosphine)rhodium chloride in acetonitrile/water at reflux.

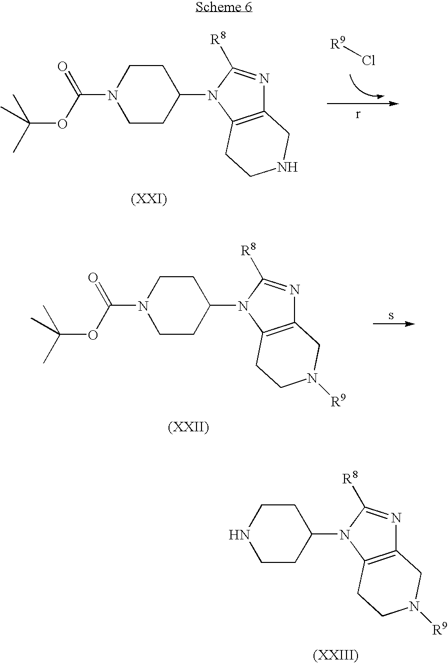

r. Amine (XXI) is reacted with R⁹Cl (such as R⁶COCl or chloroformate in the presence of a base such as triethylamine or diisopropylethylamine in a suitable solvent such as dichloromethane or toluene at a temperature between 0° C. and room temperature.
s. Removal of the Boc group may be carried out under acidic conditions e.g. hydrogen chloride dissolved in ether/methanol or ethyl acetate solution at 0° C. to room temperature or with trifluoroacetic acid in dichloromethane.

Monocyclic piperidine Hets of general formula (IV) can be formed using the principles set out in schemes 7 to 9, and 14 to 16 below with appropriate modification

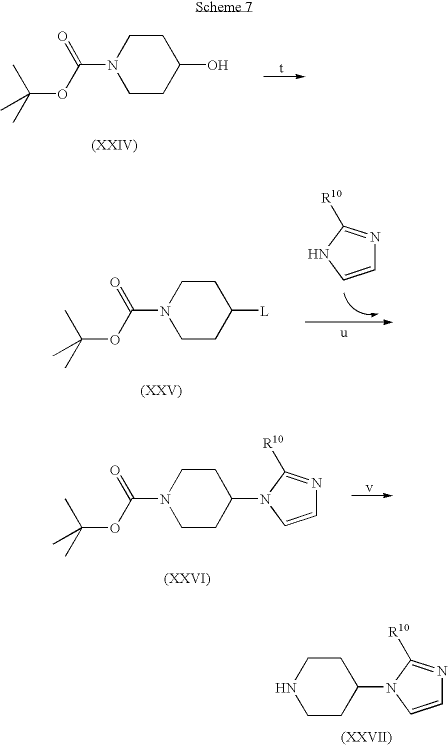

Where R¹⁰ is as defined above.

L represents a suitable leaving group such as mesylate, tosylate, chloride or bromide and is preferably mesylate.
t. The alcohol group of (XXIV) is converted to a leaving group such as a mesylate, tosylate, chloride, bromide or iodide. For example, the mesylate may be formed using methane sulphonyl chloride in the presence of a suitable base such as triethylamine or diisopropylethylamine in a solvent such as dichloromethane at a temperature between −78° C. and 0° C. and preferably around 0° C.
u. (XXV) is refluxed with an imidazole in a suitable solvent such as acetonitrile in the presence of a base such as potassium carbonate or cesium carbonate.
v. Removal of the Boc group may be carried out under acidic conditions e.g. hydrogen chloride dissolved in ether/methanol or ethyl acetate solution at 0° C. to room temperature or with trifluoroacetic acid in dichloromethane.

Examples of further routes for forming the piperidine Hets of general formula (IV) are illustrated more specifically in schemes 8 and 9.

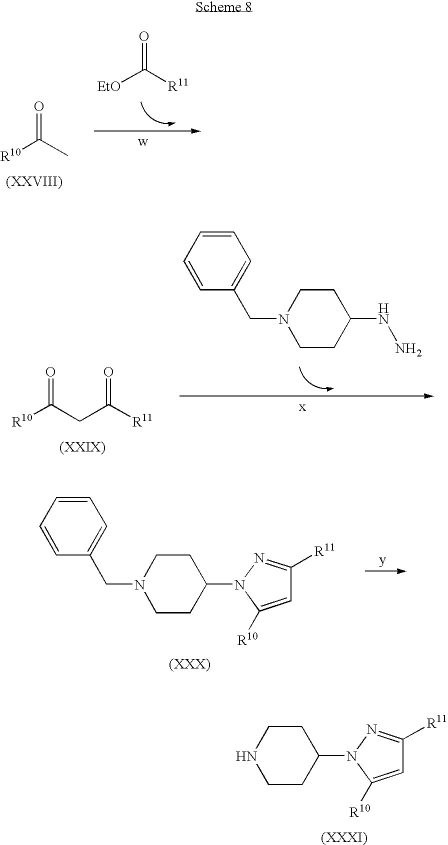

w. An alkylacetate and ketone (XXVIII) are reacted together in the presence of a suitable base such as potassium tert-butoxide in a suitable solvent such as THF at a temperature of between 0° C. and reflux (preferably around 60° C.).
x. 1-benzyl-4-hydrazinopiperidine is reacted with diketone (XXIX) in a solvent such as ethanol at a suitable temperature, typically room temperature.
y. Removal of the benzyl group is carried out by hydrogenolysis e.g. using a suitable catalyst (palladium hydroxide, palladium on carbon) under a hydrogen atmosphere in a suitable solvent such as methanol or ethanol or using ammonium formate and palladium hydroxide in a suitable solvent such as methanol or ethanol under reflux.

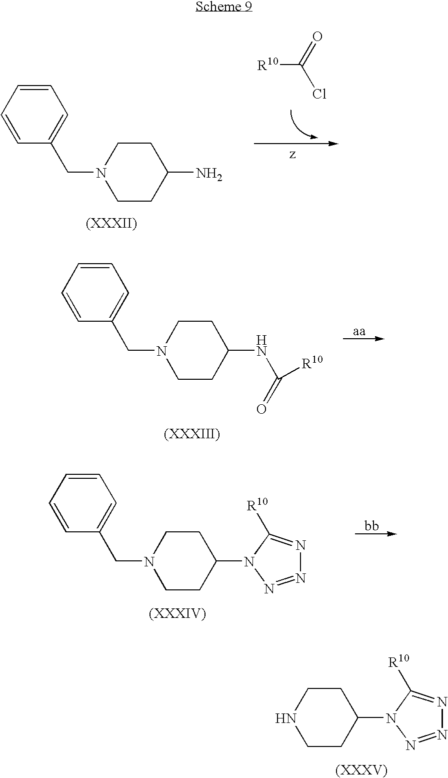

z. Amine (XXXII) is reacted with R¹⁰COCl in the presence of a base such as triethylamine or diisopropylethylamine in a suitable solvent such as dichloromethane or toluene at a temperature between 0° C. and room temperature.
aa. Amide (XXXIII) is reacted with a chlorinating agent such as phosphorus pentachloride in a solvent such as dichloromethane at a temperature of 0° C. to room temperature. The mixture is cooled to low temperature (preferentially around −5° C.) and trimethylsilylazide is added. The mixture is allowed to warm to room temperature.
bb. Removal of the benzyl group is carried out by hydrogenolysis e.g. using a suitable catalyst (palladium hydroxide, palladium on carbon) under a hydrogen atmosphere in a suitable solvent such as methanol or ethanol or using ammonium formate and palladium hydroxide in a suitable solvent such as methanol or ethanol under reflux.

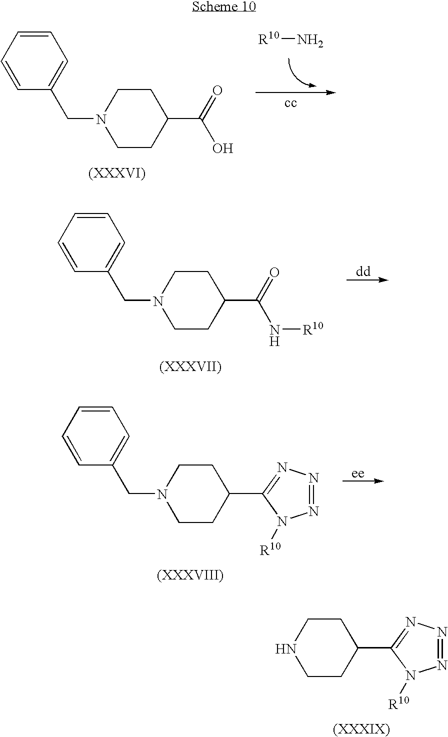

cc. Acid (XXXVI) is reacted with R¹⁰NH₂ in the presence of a coupling agent such as EDCl.HCl HBTU, HATU, DCC or preferentially EDCl.MeI in a suitable solvent such as dichloromethane or DMF. In the presence of EDCl.HCl or EDCl.MeI, HOBT is optionally added. The reaction is typically carried out at room temperature.
dd. Amide (XXXVII) is reacted with a chlorinating agent such as phosphorus pentachloride in a solvent such as dichloromethane at a temperature of 0° C. to room temperature. The mixture is cooled to low temperature (preferentially around −5° C.) and trimethylsilylazide is added. The mixture is allowed to warm to room temperature.
ee. Removal of the benzyl group is carried out by hydrogenolysis e.g. using a suitable catalyst (palladium hydroxide, palladium on carbon) under a hydrogen atmosphere in a suitable solvent such as methanol or ethanol or using ammonium formate and palladium hydroxide in a suitable solvent such as methanol or ethanol under reflux.

Conversion from an tetrahydroimadazopyridine N substituted CO₂R⁶ to a substituted COR⁶ is illustrated in scheme 11. Compounds (XLII) may then be deprotected as shown generally in scheme 1(b) to form a compound of formula (V).

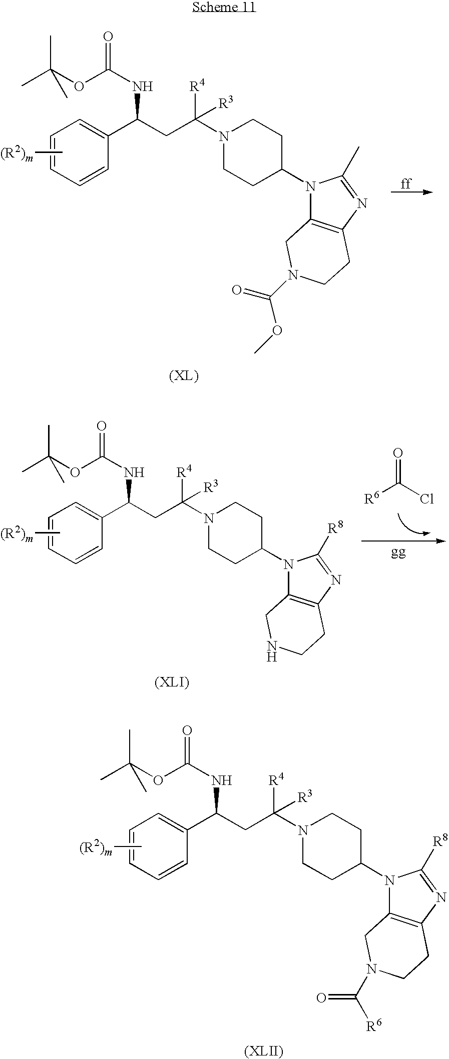

ff. Methyl carbamate (XL) is reacted with a suitable base such as sodium or potassium hydroxide in a suitable solvent such as isopropanol at elevated temperature preferentially reflux to give amine (XLI).
gg. Amine (XLI) is reacted with an acid chloride or chloroformate in the presence of a base such as triethylamine or diisopropylethylamine in a suitable solvent such as dichloromethane or toluene at a temperature between 0° C. and room temperature.
For substitution of the urea (CONR⁶R⁷) on the tetrahyroimadazole, the R⁶COCl in step gg would be replaced by NR⁶R⁷COCl.

The compounds of formula (III) may be prepared according to Scheme 12, wherein R^{a} represents hydrogen or a C₁-C₄ alkyl group (preferably methyl or ethyl),

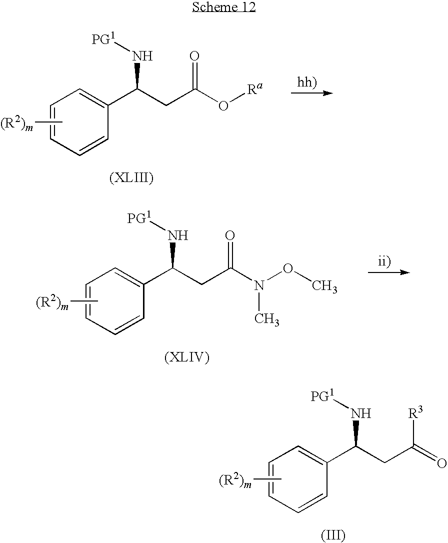

hh) Weinreb amide (XLIV) is formed as follows:
When R^{a} is an alkyl group, ester (XLIII) is reacted with N,O-dimethylhydroxylamine hydrochloride and a Grignard reagent, preferentially iso-propylmagnesium bromide or chloride, in a solvent such as THF at low temperature (typically around −10° C.).
When R^{a} is a hydrogen, acid (XLIII) (typically formed from ester (XLIII), where R^{a} is alkyl, by hydrolysis under basic conditions such as with lithium hydroxide or sodium hydroxide in MeOH or THF/water) is reacted with N,O-dimethylhydroxylamine hydrochloride under standard amide coupling conditions. For example, the reaction may be carried out in the presence of a coupling agent such as EDCl.HCl, EDCl.MeI, HBTU, HATU, DCC and a base such as Et₃N or DIPEA in a suitable solvent such as DCM or DMF. In the presence of EDCl.HCl or EDCl.MeI, HOBT is also added. The reaction is typically carried out at rt.
ii) Alkyl ester (III) is formed by reaction of Weinreb amide (XLIV) with an (R³) Grignard reagent such as methylmagnesium bromide or methylmagnesium chloride or with an (R³) lithium such as methyllithium at low temperature (typically −78° C.) in a solvent such as THF or diethylether.

The compounds of formula (VI), may be prepared as shown in Scheme 13, wherein X represents a leaving group such as Cl, Br or OH, and R^{a} is as defined in scheme 12.

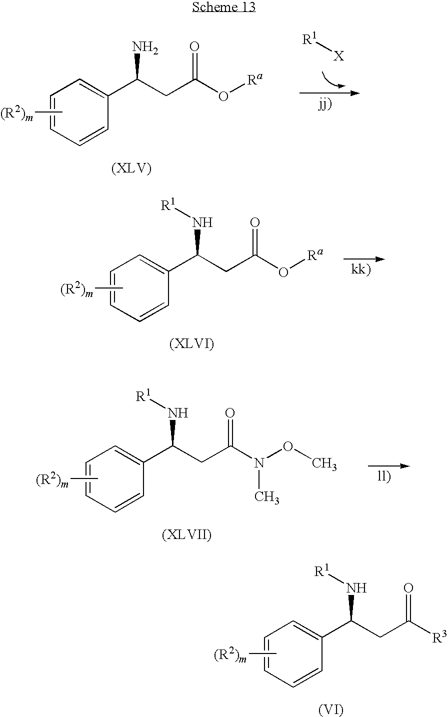

jj) Conversion of (XX) to (XXI) may be accomplished using standard amide formation conditions:
When X═Cl, (XX) is reacted with the R¹Cl in the presence of a base such as Et₃N or DIPEA in a suitable solvent such as DCM or toluene at a temperature between 0° C. and rt.
When X═OH, (XX) is reacted with R¹OHin the presence of a coupling agent such as EDCl.HCl, EDCl.MeI, HBTU, HATU, DCC in a suitable solvent such as DCM or DMF. In the presence of EDCl.HCl or EDCl.MeI, HOBT is optionally added. In the case where the amine is present as a hydrochloride salt, a suitable base such as Et₃N or DIPEA is also used. The reaction is typically carried out at rt.
kk) (XLVII) may be formed by reaction of (XLVI) with N,O-dimethylhydroxylamine hydrochloride and a Grignard reagent, preferentially iso-propylmagnesium bromide or chloride, in a solvent such as THF at −10° C.
ll) (VI) may be formed by reaction of (XLVI) with an (R³) Grignard reagent such as methylmagnesium bromide or methylmagnesium chloride or with an (R³)lithium reagent such as methyllithium at −78° C. in a solvent such as THF or diethylether.

Piperidine oxadiazole compounds of general formula (IV) can be prepared according to schemes 14 to 16 below.

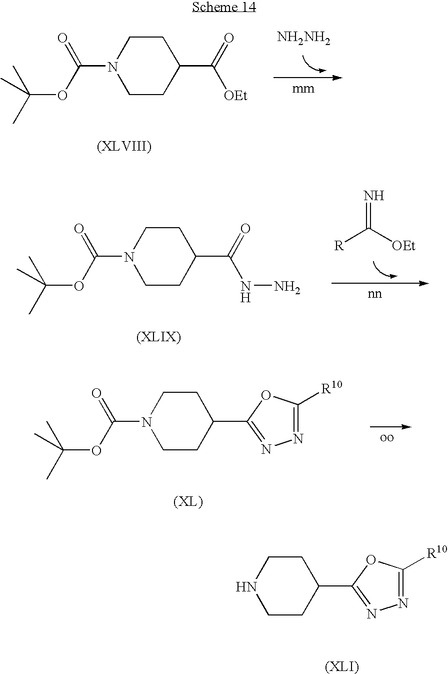

mm. Ester (XLVIII) is heated typically to reflux with hydrazine hydrate in a suitable solvent such as methanol or ethanol.
nn. (XLIX) is heated typically to reflux with an imidate in a suitable solvent such as ethanol to give the 1,3,4-triazole (XL).
oo. Removal of the Boc group may be carried out under acidic conditions e.g. hydrogen chloride dissolved in ether/methanol or ethyl acetate solution at 0° C. to room temperature or with trifluoroacetic acid in dichloromethane.

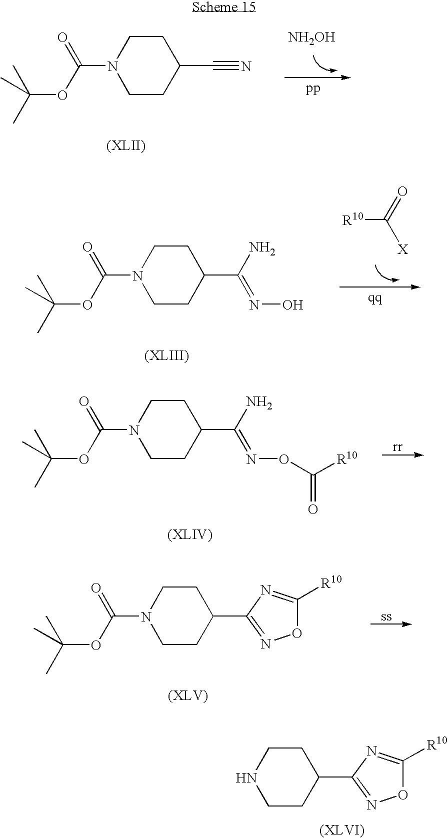

pp. Nitrile (XLII) is refluxed with hydroxylamine hydrochloride in the presence of a suitable base such as sodium carbonate in a suitable solvent such as methanol/water.

qq. (XLIII) is reacted with an acid chloride in a suitable solvent such as dichloromethane in the presence of a suitable base such as triethylamine or diisopropylethylamine optionally in the presence of catalytic 4-dimethylaminopyridine at a temperature of 0° C. to room temperature. Alternatively, (XLIII) is reacted with an acid in the presence of a suitable coupling agent such as CDI in a suitable solvent such as dichloromethane at a suitable temperature typically room temperature.
rr. (XLIV) is refluxed in a suitable solvent such as dioxane or toluene to effect ring closure.
ss. Removal of the Boc group may be carried out under acidic conditions e.g. hydrogen chloride dissolved in ether/methanol or ethyl acetate solution at 0° C. to room temperature or with trifluoroacetic acid in dichloromethane.

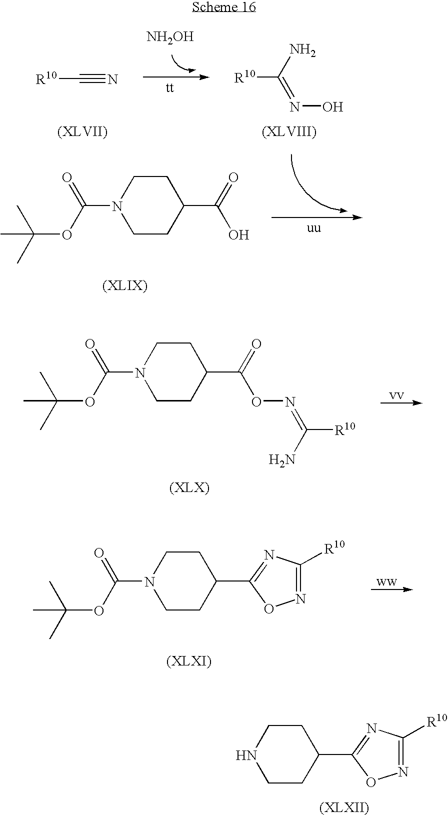

tt. Nitrile (XLVII) is refluxed with hydroxylamine hydrochloride in the presence of a suitable base such as sodium carbonate in a suitable solvent such as methanol/water.
uu. (XLVIII) is coupled with acid (XLIX) in the presence of a suitable coupling agent such as CDI in a suitable solvent such as dichloromethane at a suitable temperature typically room temperature.
vv. (XLX) is refluxed in a suitable solvent such as dioxane or toluene to effect ring closure.
ww. Removal of the Boc group may be carried out under acidic conditions e.g. hydrogen chloride dissolved in ether/methanol or ethyl acetate solution at 0° C. to room temperature or with trifluoroacetic acid in dichloromethane

It will be appreciated by those skilled in the art that certain of the procedures described in the schemes for the preparation of compounds of formula (I) or intermediates thereto may not be applicable to some of the possible substituents.

It will be further appreciated by those skilled in the art that it may be necessary or desirable to carry out the transformations described in the schemes in a different order from that described, or to modify one or more of the transformations, to provide the desired compound of formula (I).

The compounds of formula (I) and their pharmaceutically acceptable salts, solvates and derivatives are useful because they have pharmacological activity in animals, including humans. More particularly, they are useful in the treatment of a disorder in which the modulation, in particular antagonism of CCR5 receptors is implicated. Disease states of particular interest include HIV, retroviral infections genetically related to HIV, AIDS, inflammatory diseases, autoimmune diseases and pain.

The compounds of this invention may be used for treatment of respiratory disorders, including adult respiratory distress syndrome (ARDS), bronchitis, chronic bronchitis, chronic obstructive pulmonary disease, cystic fibrosis, asthma, emphysema, rhinitis, chronic sinusitis, sarcoidosis, farmer's lung, nasal polyposis, fibroid lung or idiopathic interstitial pneumonia.

Other conditions that may be treated are those triggered, affected or are in any other way correlated with T-cell trafficking in different organs. It is expected that the compounds of this invention may be useful for the treatment of such conditions and in particular, but not limited to, conditions for which a correlation with CCR5 or CCR5 chemokines has been established, and more particularly, but not limited to, the following: multiple sclerosis; Behcet's disease, Sjogren's syndrome or systemic sclerosis; arthritis, such as rheumatoid arthritis, spondyloarthropathies, gouty arthritis, osteoarthritis, systemic lupus erythematosus, and juvenile arthritis; and graft rejection, in particular, but not limited to, solid organ transplants, such as heart, lung, liver, kidney and pancreas transplants (e.g. kidney and lung allografts), and graft versus host rejection; inflammatory bowel disease, including Crohn's disease and ulcerative colitis; inflammatory lung conditions; endometriosis; renal diseases, such as glomerular disease (e.g. glomerulonephritis); fibrosis, such as liver, pulmonary and renal fibrosis; encephalitis, such as HIV encephalitis; chronic heart failure; myocardial infarction; hypertension; stroke; ischaemic heart disease; atherosclerotic plaque; restenosis; obesity; psoriasis; atopic dermatitis; CNS diseases, such as AIDS related dementias and Alzheimer's disease; anaemia; chronic pancreatitis; Hashimoto's thyroiditis; type I diabetes; cancer, such as non-Hodgkin's lymphoma, Kaposi's sarcoma, multiple myeloma, melanoma and breast cancer; pain, such as nociceptive pain and neuropathic pain (e.g. peripheral neuropathic pain); and stress response resulting from surgery, infection, injury or other traumatic insult.

Infectious diseases where modulation of the CCR5 receptor is implicated include acute and chronic hepatitis B Virus (HBV) and hepatitis C Virus (HCV) infection; bubonic, septicemic, and pneumonic plague; pox virus infection, such as smallpox; toxoplasmosis infection; mycobacterium infection; trypanosomal infection such as Chagas' Disease; pneumonia; and cytosporidiosis.

The following set out possible applications of chemokines and chemokine receptor blockers: Cascieri, M. A., and Springer, M. S., “The chemokine/chemokine receptor family: potential and progress for therapeutic intervention”, Curr. Opin. Chem. Biol., 4(4), 420-7 (August 2000); Ribeiro and Horuk, “The Clinical Potential of Chemokine Receptor Antagonists”, Pharmacology and Therapeutics 107 (2005) p44-58.

Accordingly, in another aspect the invention provides a compound of formula (I) or a pharmaceutically acceptable salt, solvate or derivative thereof for use as a medicament.

In another aspect the invention provides a compound of formula (I) or a pharmaceutically acceptable salt, solvate or derivative thereof, for the treatment of a disorder in which the modulation of CCR5 receptors is implicated.

In another aspect the invention provides the use of a compound of formula (I) or of a pharmaceutically acceptable salt, solvate or derivative thereof, in the manufacture of a medicament for the treatment of a disorder in which the modulation of CCR5 receptors is implicated.

In another aspect the invention provides a method of treatment of a disorder in which the modulation of CCR5 receptors is implicated which comprises administering to a patient in need thereof (e.g a human patient or an animal patient) a therapeutically effective amount of a compound of formula (I) or a pharmaceutically acceptable salt, solvate or derivative thereof.

The compounds of formula (I) are useful in the treatment of the diseases, disorders or conditions mentioned above; diseases of particular interest include HIV, retroviral infections genetically related to HIV, AIDS, inflammatory diseases, autoimmune diseases and pain.

For the avoidance of doubt, references herein to “treatment” include references to curative, palliative and prophylactic treatment.

Compounds of formula (I) intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of formula (I) or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form. Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995), incorporated herein by reference.

Suitable modes of administration include oral, parenteral, topical, inhaled/intranasal, rectal/intravaginal, and ocular/aural administration.

The compounds of formula (I) may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of formula (I) may also be used in fast-dissolving, fast-disintegrating dosage forms such as those described in Expert Opinion in Therapeutic Patents, 11 (6), 981-986, by Liang and Chen (2001), incorporated herein by reference.

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose.

Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980), incorporated herein by reference.

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864, incorporated herein by reference. Details of other suitable release technologies such as high energy dispersions and osmotic and coated particles are to be found in Pharmaceutical Technology On-line, 25(2), 1-14, by Verma et al (2001), incorporated herein by reference. The use of chewing gum to achieve controlled release is described in WO 00/35298, incorporated herein by reference.

The compounds of formula (I) may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of formula (I) may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

The compounds of formula (I) may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999), incorporated herein by reference.

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of formula (I) can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 μg to 10 mg of the compound of the invention. The overall daily dose will typically be in the range 1 μg to 200 mg which may be administered in a single dose or, more usually, as divided doses throughout the day.

The compounds of formula (I) may be administered rectally or vaginally, for example, in the form of a suppository, pessary, vaginal ring or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of formula (I) may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of formula (I) may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148, incorporated herein by reference.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, having a weight of about 65 to 70 kg, the total daily dose of a compound of the invention is typically in the range 1 to 10,000 mg, such as 10 to 1,000 mg, for example 25 to 500 mg, depending, of course, on the mode of administration, the age, condition and weight of the patient, and will in any case be at the ultimate discretion of the physician. The total daily dose may be administered in single or divided doses.

Accordingly in another aspect the invention provides a pharmaceutical composition comprising a compound of formula (I) or a pharmaceutically acceptable salt, solvate or derivative thereof together with one or more pharmaceutically acceptable excipients, diluents or carriers.

The compounds of formula (I) and their pharmaceutically acceptable salts, solvates and derivatives may be administered alone or as part of a combination therapy. Thus included within the scope of the present invention are embodiments comprising co-administration of, and compositions which contain, in addition to a compound of the invention, one or more additional therapeutic agents.

Such multiple drug regimens, often referred to as combination therapy, may be used in the treatment and prevention of any of the diseases or conditions mediated by or associated with CCR5 chemokine receptor modulation, particularly infection by human immunodeficiency virus, HIV. The use of such combination therapy is especially pertinent with respect to the treatment and prevention of infection and multiplication of the human immunodeficiency virus, HIV, and related pathogenic retroviruses within a patient in need of treatment or one at risk of becoming such a patient. The ability of such retroviral pathogens to evolve within a relatively short period of time into strains resistant to any monotherapy which has been administered to said patient is well known in the literature. A recommended treatment for HIV is a combination drug treatment called Highly Active Anti-Retroviral Therapy, or HAART. HAART combines three or more HIV drugs. Thus, the methods of treatment and pharmaceutical compositions of the present invention may employ a compound of the invention in the form of monotherapy, but said methods and compositions may also be used in the form of combination therapy in which one or more compounds of formula (I) are co-administered in combination with one or more additional therapeutic agents such as those described in detail further herein.

The therapeutic agents that may be used in combination with the compounds of the present invention include, but are not limited to, those useful as HIV protease inhibitors (PIs), non-nucleoside reverse transcriptase inhibitors (NNRTIs), nucleoside/nucleotide reverse transcriptase inhibitors (NRTIs), CCR5 antagonists, agents which inhibit the interaction of gp120 with CD4, other agents which inhibit the entry of HIV into a target cell (such as fusion inhibitors), inhibitors of HIV integrase, RNaseH inhibitors, prenylation inhibitors, maturation inhibitors which act by interfering with production of the HIV capsid protein, compounds useful as anti-infectives, and others as described below.

It will be appreciated by a person skilled in the art, that a combination drug treatment, as described herein above, may comprise two or more compounds having the same, or different, mechanism of action. Thus, by way of illustration only, a combination may comprise a compound of the invention and: one or more NRTIs; one or more NRTIs and a PI; one or more NRTIs and another CCR5 antagonist; a PI; a PI and an NNRTI; an NNRTI; and so on.

Examples of PIs include, but are not limited to, amprenavir (141W94), CGP-73547, CGP-61755, DMP-450 (mozenavir), nelfinavir, ritonavir, saquinavir (invirase), lopinavir, TMC-126, atazanavir, palinavir, GS-3333, KN I-413, KNI-272, LG-71350, CGP-61755, PD 173606, PD 177298, PD 178390, PD 178392, U-140690, ABT-378, DMP-450, AG-1776, MK-944, VX-478, indinavir, tipranavir, TMC-114, DPC-681, DPC-684, fosamprenavir calcium (Lexiva), benzenesulfonamide derivatives disclosed in WO 03/053435, R-944, Ro-03-34649, VX-385, GS-224338, OPT-TL3, PL-100, PPL-100, SM-309515, AG-148, DG-35-VIII, DMP-850, GW-5950X, KNI-1039, L-756423, LB-71262, LP-130, RS-344, SE-063, UIC-94-003, Vb-19038, A-77003, BMS-182193, BMS-186318, SM-309515, JE-2147, GS-9005.

Examples of NRTIs include, but are not limited to, abacavir, GS-840, lamivudine, adefovir dipivoxil, beta-fluoro-ddA, zalcitabine, didanosine, stavudine, zidovudine, tenofovir disoproxil fumarate, amdoxovir (DAPD), SPD-754, SPD-756, racivir, reverset (DPC-817), MIV-210 (FLG), beta-L-Fd4C (ACH-126443), MIV-310 (alovudine, FLT), dOTC, DAPD, entecavir, GS-7340, emtricitabine (FTC).

Examples of NNRTIs include, but are not limited to, efavirenz, HBY-097, nevirapine, TMC-120 (dapivirine), TMC-125, etravirine, delavirdine, DPC-083, DPC-961, capravirine, rilpivirine, 5-{[3,5-Diethyl-1-(2-hydroxyethyl)-1H-pyrazol-4-yl]oxy}isophthalonitrile or pharmaceutically acceptable salts, solvates or derivatives thereof; GW-678248, GW-695634, MIV-150, calanolide, and tricyclic pyrimidinone derivatives as disclosed in WO 03/062238.

Examples of CCR5 antagonists include, but are not limited to, TAK-77; SC-351125; ancriviroc (formely known as SCH-C); vicriviroc (formely known as SCH-D); PRO-140; maraviroc; apliviroc (formely known as GW-873140, Ono-4128, AK-602); AMD-887; CMPD-167; methyl 1-endo{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate or pharmaceutically acceptable salts, solvates or derivatives thereof; methyl 3-endo{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carboxylate or pharmaceutically acceptable salts, solvates or derivatives thereof; ethyl 1-endo{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate or pharmaceutically acceptable salts, solvates or derivatives thereof; and N-{(1S)-3-[3-endo-(5-Isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide) or pharmaceutically acceptable salts, solvates or derivatives thereof.

Examples of entry and fusion inhibitors include, but are not limited to, BMS-806, BMS-488043, 5-{(1S)-2-[(2R)-4-Benzoyl-2-methyl-piperazin-1-yl]-1-methyl-2-oxo-ethoxy}-4-methoxy-pyridine-2-carboxylic acid methylamide and 4-{(1S)-2-[(2R)-4-Benzoyl-2-methyl-piperazin-1-yl]-1-methyl-2-oxo-ethoxy}-3-methoxy-N-methyl-benzamide, enfuvirtide (T-20), sifuvirtide, SP-01A, T1249, PRO542, AMD-3100, soluble CD4, compounds disclosed in JP 2003171381, and compounds disclosed in JP 2003119137.

Examples of inhibitors of HIV integrase include, but are not limited to, L-000870810 GW-810781, 1,5-naphthyridine-3-carboxamide derivatives disclosed in WO 03/062204, compounds disclosed in WO 03/047564, compounds disclosed in WO 03/049690, and 5-hydroxypyrimidine-4-carboxamide derivatives disclosed in WO 03/035076, MK-0518 (5-(1,1-dioxo-1,2-thiazinan-2-yl)-N-(4-fluorobenzyl)-8-hydroxy-1,6-naphthyridine-7-carboxamide- disclosed in WO 03016315), GS-9137 (JTK-303).

Examples of prenylation inhibitors include, but are not limited to, HMG CoA reductase inhibitors, such as statins (e.g. atorvastatin).

Examples of maturation inhibitors include 3-O-(3′,3′-dimethylsuccinyl) betulic acid (otherwise known as PA-457) and alpa-HGA.

Anti-infectives that may be used in combination with the compounds of the present invention include antibacterials and antifungals. Examples of antibacterials include, but are not limited to, atovaquone, azithromycin, clarithromycin, trimethoprim, trovafloxacin, pyrimethamine, daunorubicin, clindamycin with primaquine, fluconazole, pastill, ornidyl, eflornithine pentamidine, rifabutin, spiramycin, intraconazole-R51211, trimetrexate, daunorubicin, recombinant human erythropoietin, recombinant human growth hormone, megestrol acetate, testerone, and total enteral nutrition. Examples of antifungals include, but are not limited to, anidulafungin, C31G, caspofungin, DB-289, fluconazaole, itraconazole, ketoconazole, micafungin, posaconazole, and voriconazole.

There is also included within the scope the present invention, combinations of a compound of formula (I), or a pharmaceutically acceptable salt, solvate or derivative thereof, together with one or more additional therapeutic agents independently selected from the group consisting of:

Proliferation inhibitors, e.g. hydroxyurea.

Immunomodulators, such as AD-439, AD-519, alpha interferon, AS-101, bropirimine, acemannan, CL246,738, EL10, FP-21399, gamma interferon, granulocyte macrophage colony stimulating factor (e.g. sargramostim), IL-2, immune globulin intravenous, IMREG-1, IMREG-2, imuthiol diethyl dithio carbamate, alpha-2 interferon, methionine-enkephalin, MTP-PE, remune, rCD4, recombinant soluble human CD4, interferon alfa-2, SK&F106528, soluble T4 thymopentin, tumor necrosis factor (TNF), tucaresol, recombinant human interferon beta, interferon alfa n-3.

Tachykinin receptor modulators (e.g. NK1 antagonists) and various forms of interferon or interferon derivatives.

Other chemokine receptor agonists/antagonists such as CXCR4 antagonists (e.g AMD070 and AMD3100) or CD4 antagonists (e.g. TNX-355).

Agents which substantially inhibit, disrupt or decrease viral transcription or RNA replication such as inhibitors of tat (transcriptional trans activator) or nef (negative regulatory factor).

Agents which substantially inhibit, disrupt or decrease translation of one or more proteins expressed by the virus (including, but not limited to, down regulation of protein expression or antagonism of one or more proteins) other than reverse transcriptase, such as Tat or Nef.

Agents which influence, in particular down regulate, CCR5 receptor expression; chemokines that induce CCR5 receptor internalisation such MIP-1α, MIP-1β, RANTES and derivatives thereof; examples of such agents include, but are not limited to, immunosupressants, such as calcineurin inhibitors (e.g. tacrolimus and cyclosporin A); steroids; agents which interfere with cytokine production or signalling, such as Janus Kinase (JAK) inhibitors (e.g. JAK-3 inhibitors, including 3-{(3R,4R)-4-methyl-3-[methyl-(7H-pyrrolo[2,3-d]pyrimidin-4-yl)-amino]-piperidin-1-yl}-3-oxo-propionitrile) and pharmaceutically acceptable salts, solvates or derivatives thereof; cytokine antibodies (e.g. antibodies that inhibit the interleukin-2 (IL-2) receptor, including basiliximab and daclizumab);

Agents which interfere with cell activation or cell cycling, such as rapamycin.

In addition to the requirement of therapeutic efficacy, which may necessitate the use of therapeutic agents in addition to the compounds of formula (I), there may be additional rationales which compel or highly recommend the use of a combination of a compound of the invention and another therapeutic agent, such as in the treatment of diseases or conditions which directly result from or indirectly accompany the basic or underlying CCR5 chemokine receptor modulated disease or condition. For example, where the basic CCR5 chemokine receptor modulated disease or condition is HIV infection and multiplication it may be necessary or at least desirable to treat Hepatitis C Virus (HCV), Hepatitis B Virus (HBV), Human Papillomavirus (HPV), neoplasms, and other conditions which occur as the result of the immune-compromised state of the patient being treated. Other therapeutic agents may be used with the compounds of formula (I), e.g., in order to provide immune stimulation or to treat pain and inflammation which accompany the initial and fundamental HIV infection.

Accordingly, therapeutic agents for use in combination with the compounds of formula (I) and their pharmaceutically acceptable salts, solvates and derivatives also include:

Agents useful in the treatment of hepatitis, such as interferons, pegylated interferons (e.g. peginterferon alfa-2a and peginterferon alfa-2b), long-acting interferons (e.g. albumin-interferon alfa); TLR7 inhibitors; reverse transcriptase inhibitors, such as lamivudine and emtricitabine; IMP dehydrogenase inhibitors such as ribavirin and viramidine; polymerase inhibitors (including NS5B polymerase inhibitors) such as valopicitabine, HCV-086, HCV-796 purine nucleoside analogues as disclosed in WO 05/009418, and imidazole derivatives as disclosed in WO 05/012288; alpha glucosidase inhibitors such as celgosivir; interferon enhancers such as EMZ-702; serine protease inhibitors such as BILN-2061, SCH-6, VX-950, aza-peptide-based macrocyclic derivatives as disclosed in WO 05/010029 and those disclosed in WO 05/007681; caspase inhibitors such as IDN-6566; HCV replicon inhibitors such as arylthiourea derivatives as disclosed in WO 05/007601.

Agents useful in the treatment of AIDS related Kaposi's sarcoma, such as interferons, daunorubicin, doxorubicin, paclitaxel, metallo-matrix proteases, A-007, bevacizumab, BMS-275291, halofuginone, interleukin-12, rituximab, porfimer sodium, rebimastat, COL-3.

Agents useful in the treatment of cytomegalovirus (CMV), such as fomivirsen, oxetanocin G, cidofovir, cytomegalovirus immune globin, foscarnet sodium, Isis 2922, valacyclovir, valganciclovir, ganciclovir.

Agents useful in the treatment of herpes simplex virus (HSV), such as acyclovir, penciclovir, famciclovir, ME-609.

Further combinations for use according to the invention include combination of a compound of formula (I), or a pharmaceutically acceptable salt, solvate or derivative thereof with a CCR1 antagonist, such as BX-471; a beta adrenoceptor agonist, such as salmeterol; a corticosteroid agonist, such fluticasone propionate; a LTD4 antagonist, such as montelukast; a muscarinic antagonist, such as tiotropium bromide; a PDE4 inhibitor, such as cilomilast or roflumilast; a COX-2 inhibitor, such as celecoxib, valdecoxib or rofecoxib; an alpha-2-delta ligand, such as gabapentin or pregabalin; a beta-interferon, such as REBIF; a TNF receptor modulator, such as a TNF-alpha inhibitor (e.g. adalimumab).

There is also included within the scope the present invention, combinations of a compound of formula (I), or a pharmaceutically acceptable salt, solvate or derivative thereof, together with one or more additional therapeutic agents which slow down the rate of metabolism of the compound of the invention, thereby leading to increased exposure in patients. Increasing the exposure in such a manner is known as boosting. This has the benefit of increasing the efficacy of the compound of the invention or reducing the dose required to achieve the same efficacy as an unboosted dose. The metabolism of the compounds of formula (I) includes oxidative processes carried out by P450 (CYP450) enzymes, particularly GYP 3A4 and conjugation by UDP glucuronosyl transferase and sulphating enzymes. Thus, among the agents that may be used to increase the exposure of a patient to a compound of the present invention are those that can act as inhibitors of at least one isoform of the cytochrome P450 (CYP450) enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP 3A4 include, but are not limited to, ritonavir, saquinavir or ketoconazole.

In the above-described combinations, the compound of formula (I) or a pharmaceutically acceptable salt, solvate or derivative thereof and other therapeutic agent(s) may be administered, in terms of dosage forms, either separately or in conjunction with each other; and in terms of their time of administration, either simultaneously or sequentially. Thus, the administration of one component agent may be prior to, concurrent with, or subsequent to the administration of the other component agent(s).

Accordingly, in a further aspect the invention provides a pharmaceutical composition comprising a compound of formula (I) or a pharmaceutically acceptable salt, solvate or derivative thereof and one or more additional therapeutic agents.

The invention is illustrated by the following Preparations and Examples in which the following further abbreviations may be used:

0.88 ammonia=concentrated ammonium hydroxide solution
APCI=atmospheric pressure chemical ionisation
DMSO=dimethyl sulphoxide
ES=electrospray ionisation
HRMS=high resolution mass spectrum
LCMS=liquid chromatography-mass spectroscopy;
LRMS=low resolution mass spectrum
MS=mass spectrum
NMR=nuclear magnetic resonance
eq.=equivalent
h=hour
min=minute
m.p.=melting point

### Preparation 1

### N-(1-Benzylpiperidin-4-yl)-4-nitropyridin-3-amine 1-oxide

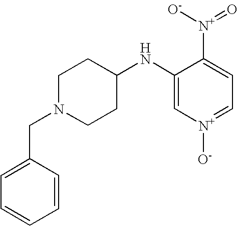

3-Fluoro-4-nitropyridine 1-oxide (33 g, 210 mmol) was added to a stirring solution of 4-amino-1-benzyl-piperidine (40 g, 210 mmol) and N,N-diisopropylethylamine (36.6 mL, 210 mmol) in acetonitrile (400 mL) at 0° C. The mixture was allowed to warm slowly to room temperature and stirred for 16 hours. The resulting precipitate was filtered off, washed with acetonitrile and then dried in vacuo to afford the title compound as a yellow solid, 62.6 g.

LRMS: m/z APCI-329 [MH]⁺

### Preparation 2

### N³-Piperidin-4-ylpyridine-3,4-diamine

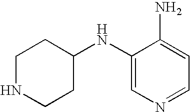

The amine of Preparation 1 (31.3 g, 95 mmol) was dissolved in a mixture of ethanol (250 mL), water (125 mL) and acetic acid (62.5 mL) and then hydrogenated at 40° C., 90 Psi over Degussa E101 5% palladium on carbon (3.12 g) for 24 hours. The cooled mixture was filtered through Arbocel® and the filtrate evaporated under reduced pressure to afford the title compound as a white solid.

LRMS: m/z APCI 193 [MH]⁺

### Preparation 3

### 3-(1-Acetylpiperidin-4-yl)-2-methyl-3H-imidazo[4,5-c]pyridine

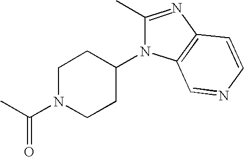

The amine of Preparation 2 (36.5 g, 190 mmol) and acetic anhydride (179 mL, 1900 mmol) were heated together at 100° C. for 24 hours. After cooling, ethanol (100 mL) was added and the mixture was concentrated under reduced pressure. The residue was dissolved in water and basified with sodium hydroxide pellets. The resulting mixture was washed with ethyl acetate (3×250 mL). The aqueous was extracted with dichloromethane (5×200 mL) and then by continuous extraction with dichloromethane. The combined dichloromethane fractions were heated with decolourising charcoal and dried (MgSO₄) to afford the title compound as a white solid, 42.3 g.

LRMS: m/z APCI-259 [MH]⁺

### Preparation 4

### 3-(1-Acetylpiperidin-4-yl)-2-methyl-4,5,6,7-tetrahydro-3H-imidazo[4,5-c]pyridine

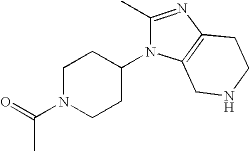

The amide of Preparation 3 (21.1 g, 81.8 mmol) was dissolved in a mixture of ethanol (330 mL) and 5N hydrochloric acid (33 mL). The mixture was hydrogenated over platinum oxide (2.6 g) at 50° C. and 50Psi for 2 days. The reaction mixture was filtered through Arbocel® and then evaporated under reduced pressure to afford the title compound as a white solid, 30.8 g

LRMS: m/z APCI-263 [MH]⁺

### Preparation 5

### Methyl 3-(1-acetylpiperidin-4-yl)-2-methyl-3,4,6,7-tetrahydro-5H-imidazo[4,5-c]pyridine-5-carboxylate

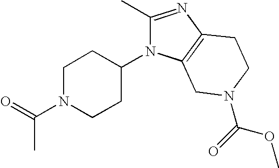

The amide of Preparation 4 (10 g, 29.8 mmol) was suspended in dichloromethane (150 mL) and cooled to −10° C. under nitrogen. Triethylamine (12.5 mL, 89.4 mmol) was added to the mixture and then after a further 10 mins, methyl chloroformate (2.3 mL, 29.8 mmol) was added dropwise maintaining the temperature below −10° C. The reaction mixture was allowed to warm to room temperature and stirred for 16 hours. Dichloromethane (150 mL) was added and the mixture was washed with water (2×150 mL), brine, dried (MgSO₄) and the solvent evaporated under reduced pressure. The residue was purified by column chromatography on silica gel, eluting with dichloromethane:methanol:0.88 ammonia (95:5:0.5) to afford a white solid, 6.1 g.

LRMS: m/z APCI-321 [MH]⁺

### Preparation 6

### Methyl 2-methyl-3-piperidin-4-yl-3,4,6,7-tetrahydro-5H-imidazo[4,5-c]pyridine-5-carboxylate

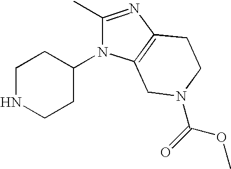

The carbamate of Preparation 5 (9.2 g, 29 mmol) and 2N hydrochloric acid (100 mL) were heated at 65° C. for 24 hours. The mixture was allowed to cool and then basified by addition of sodium hydroxide pellets. The aqueous layer was evaporated under reduced pressure to around 20 mL volume and the mixture was extracted with dichloromethane (4×150 mL). The combined fractions were dried (MgSO₄) and the solvent evaporated under reduced pressure to give a white solid, 7.4 g.

LRMS: m/z APCI-279 [MH]⁺

### Preparation 7

### Ethyl 4-[(3-nitropyridin-4-yl)amino]piperidine-1-carboxylate

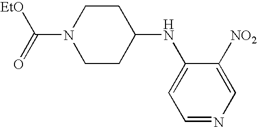

Ethyl aminopiperidine carboxylate (33.7 mL, 196.42 mmol) was dissolved in acetonitrile (240 mL) and ethoxy 3-nitropyridine hydrochloride (42.5 g, 208.2 mmol)) was added followed by triethylamine (30 mL, 208.2 mmol). The mixture was heated to go ° C. under nitrogen for 72 hours. After cooling, the solvent was removed in vacuo and the residue was dissolved in ethyl acetate (200 mL), washed with sodium hydrogen carbonate (2×200 mL), dried (MgSO₄) and the solvent removed under reduced pressure. The residue was triturated with ether to give the title compound as a yellow solid, 24 g.

LRMS: m/z APCI-295 [MH]⁺

### Preparation 8

### Ethyl 4-[(3-aminopyridin-4-yl)amino]piperidine-1-carboxylate

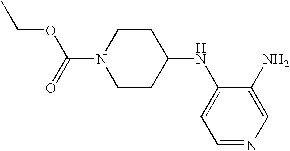

The carbamate of Preparation 7 (24 g, 81.6 mmol) was dissolved in ethanol (300 mL) and hydrogenated at room temperature, 40 Psi over 10% palladium on carbon for 16 hours. The mixture was filtered through Arbocel® and the filtrate evaporated under reduced pressure to give the title compound as a brown solid, 22 g.

LRMS: m/z APCI-265 [MH]⁺

### Preparation 9

### Ethyl 4-(2-methyl-1H-imidazo[4,5-c]pyridin-1-yl)piperidine-1-carboxylate

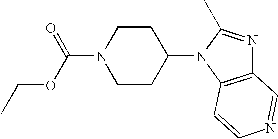

The carbamate of Preparation 8 (23 g, 87.1 mmol) was dissolved in acetic anhydride (250 mL) and the mixture was refluxed for 16 hours under nitrogen. The mixture was cooled to 0° C. and quenched by careful addition of methanol (250 mL). The solvent was removed in vacuo and the residue was partitioned between saturated sodium carbonate solution (200 mL) and dichloromethane (200 mL). The layers were separated and the aqueous was further extracted with dichloromethane (200 mL). The combined organic fractions were dried (MgSO₄) and the solvent removed under reduced pressure. The residue was triturated with acetonitrile/diisopropylether (9:1) to give the title compound as a brown solid, 12.5 g. The filtrate was evaporated under reduced pressure and the residue was purified by column chromatography on silica gel using an elution gradient of 100% dichloromethane to 90:10 dichloromethane:methanol to afford further title compound as a brown solid, 7.3 g.

LRMS: m/z APCI-289 [MH]⁺

### Preparation 10

### 2-Methyl-1-piperidin-4-yl-1H-imidazo[4,5-c]pyridine

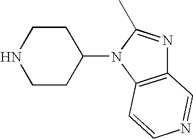

The carbamate of Preparation 9 (14.2 g, 49.12 mmol) was dissolved in ethanol (80 mL) and a solution of potassium hydroxide (9.48 g, 167.01 mmol) in water (30 mL) was added. The mixture was refluxed for 70 hours under nitrogen. After cooling to room temperature, water (200 mL) was added and the mixture was extracted with dichloromethane (4×300 mL). The combined organic fractions were dried (MgSO₄) and the solvent removed in vacuo. The residue was purified by column chromatography on silica gel using an elution gradient of 100% dichloromethane to 90:10:1 dichloromethane:methanol:0.88 ammonia to give the title compound as a solid, 8.5 g.

LRMS: m/z ES 217 [MH]⁺

### Preparation 11

### tert-Butyl 4-(2-methyl-1H-imidazo[4,5-c]pyridin-1-yl)piperidine-1-carboxylate

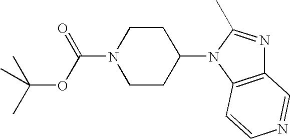

The amine of Preparation 10 (7.5 g, 34.72 mmol) was dissolved in dichloromethane (400 mL) and di-t-butyl dicarbonate (7.58 g, 34.72 mmol) was added. The mixture was stirred at room temperature for 16 hours under nitrogen and then washed with saturated sodium hydrogen carbonate solution (2×300 mL). The organic layer was dried (MgSO₄) and the solvent removed in vacuo to give the title compound as a solid, 10.9 g.

LRMS: m/z ES 317 [MH]⁺

### Preparation 12

### Ethyl 4-(5-allyl-2-methyl-4,5,6,7-tetrahydro-1H-imidazo[4,5-c]pyridin-1-yl)piperidine-1-carboxylate

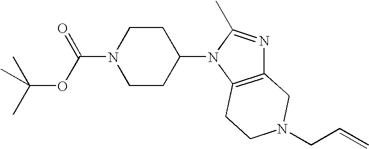

The carbamate of Preparation 11 (9.5 g, 30.1 mmol), allyl bromide (2.6 mL, 30.1 mmol) and sodium iodide (4.5 g, 30.1 mmol) were dissolved in ethanol (250 mL) and refluxed under nitrogen for 16 hours. The mixture was allowed to cool to room temperature and sodium borohydride (2.28 g, 60.1 mmol) was added portionwise. After stirring for 1 hour, water (100 mL) was added and the solvent was removed in vacuo. The residue was dissolved in ethyl acetate (200 mL), washed with water (2×200 mL), dried (MgSO₄) and the solvent removed under reduced pressure to give the title compound as an oil, 10.5 g.

LRMS: m/z APCI-361 [MH]⁺

### Preparation 13

### tert-Butyl 4-(2-methyl-4,5,6,7-tetrahydro-1H-imidazo[4,5-c]pyridin-1-yl)piperidine-1-carboxylate

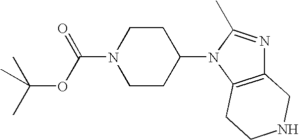

The carbamate of Preparation 12 (10.5 g, 29.16 mmol), 1,3-dimethyl barbituric acid (13.65 g, 87.5 mmol) and tetrakis(triphenylphosphine)palladium(0) (505 mg, 0.44 mmol) were dissolved in dichloromethane (500 mL) and the mixture was refluxed for 4 hours. After cooling, the solvent was removed under reduced pressure and the residue was triturated with diethylether. The resulting solid was purified by column chromatography on silica gel using an elution gradient of dichloromethane:methanol:0.88 ammonia (90:10:1 to 80:20:3) to give the title compound as a white solid, 6.4 g.

LRMS: m/z APCI-321 [MH]⁺

### Preparation 14

### Methyl 1-[1-(tert-butoxycarbonyl)piperidin-4-yl]-2-methyl-1,4,6,7-tetrahydro-5H-imidazo[4,5-c]pyridine-5-carboxylate

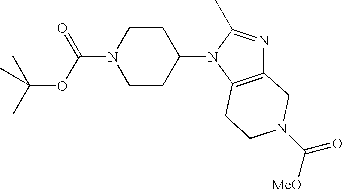

The carbamate of Preparation 13 (6.4 g, 20 mmol) was dissolved in dichloromethane (250 mL) at 0° C. under nitrogen and triethylamine (4.18 mL, 30 mmol) was added. Methyl chloroformate (1.55 mL, 20 mmol) was added dropwise maintaining the temperature below 4° C. After 2 hours at 0° C., the mixture was warmed to room temperature and stirred for a further 16 hours. The mixture was washed with saturated sodium hydrogen carbonate solution (2×200 mL), the organic layer was dried (MgSO₄) and the solvent removed under reduced pressure to give the title compound as a solid, 7 g.

LRMS: m/z APCI-379 [MH]⁺

### Preparation 15

### Methyl 2-methyl-1-piperidin-4-yl-1,4,6,7-tetrahydro-5H-imidazo[4,5-c]pyridine-5-carboxylate

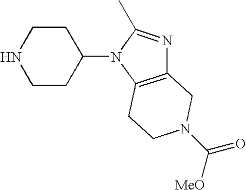

The carbamate of Preparation 14 (7 g, 18.5 mmol) was dissolved in ethyl acetate (100 mL) and 2N ethereal hydrochloric acid (37 mL) was added. The mixture was stirred at room temperature for 16 hours and then the mixture was concentrated in vacuo to give the title compound as a white solid, 5.9 g.

LRMS: m/z APCI-279 [MH]+

### Preparation 16

### Ethyl 1-[1-(tert-butoxycarbonyl)piperidin-4-yl]-2-methyl-1,4,6,7-tetrahydro-5H-imidazo[4,5-c]pyridine-5-carboxylate

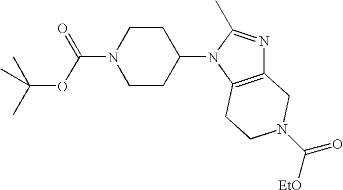

The carbamate of Preparation 13 (2.88 g, 9 mmol) was dissolved in dichloromethane (250 mL) at 0° C. under nitrogen and triethylamine (1.88 mL, 13.5 mmol) was added. Ethyl chloroformate (0.86 mL, 9 mmol) was added dropwise maintaining the temperature below 4° C. The mixture was allowed to warm to room temperature and stirred for a further 16 hours. The mixture was washed with saturated sodium hydrogen carbonate solution (2×50 mL), the organic layer was dried (MgSO₄) and the solvent removed under reduced pressure to give the title compound as a brown solid, 3.03 g.

LRMS: m/z APCI-393 [MH]+

### Preparation 17

### tert-Butyl 4-(5-acetyl-2-methyl-4,5,6,7-tetrahydro-1H-imidazo[4,5-c]pyridin-1-yl)piperidine-1-carboxylate

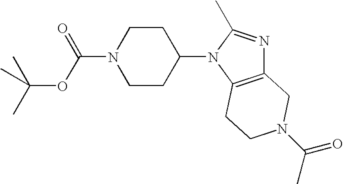

The carbamate of Preparation 13 (2.88 g, 9 mmol) was dissolved in dichloromethane (250 mL) at 0° C. under nitrogen and triethylamine (1.88 mL, 13.5 mmol) was added. Acetyl chloride (0.64 mL, 9 mmol) was added dropwise and the mixture was allowed to warm to room temperature and stirred for a further 16 hours. The mixture was washed with saturated sodium hydrogen carbonate solution (2×50 mL), the organic layer was dried (MgSO₄) and the solvent removed under reduced pressure to give the title compound as a brown solid, 3.02 g (93%).

LRMS: m/z APCI-363 [MH]⁺

### Preparation 18

### Ethyl 2-methyl-1-piperidin-4-yl-1,4,6,7-tetrahydro-5H-imidazo[4,5-c]pyridine-5-carboxylate

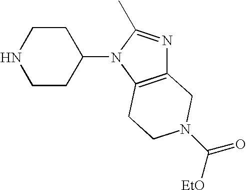

The procedure of Preparation 15 was followed reacting the carbamate of Preparation 16 with ethereal hydrogen chloride to give the title compound as the hydrochloride salt which was converted to the free-base as a colourless oil in 97% yield.

LRMS: m/z APCI-293 [MH]⁺

### Preparation 19

### 5-Acetyl-2-methyl-1-piperidin-4-yl-4,5,6,7-tetrahydro-1H-imidazo[4,5-c]pyridine

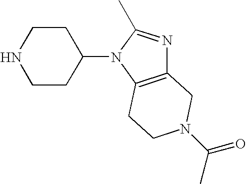

The procedure of Preparation 15 was followed reacting the compound of Preparation 17 with ethereal hydrogen chloride to give the title compound as the hydrochloride salt which was converted to the free-base as a colourless oil (80%).

LRMS: m/z APCI-263 [MH]⁺

### Preparation 20

### 4-(2-isoPropyl-1H-imidazol-1-yl)piperidine

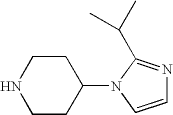

Methanesulphonyl chloride (0.27 mL, 3.4 mmol) was added dropwise to a stirring mixture of 1-tertbutoxycarbonyl-4-hydroxypiperidine (457 mg, 2.27 mmol) and triethylamine (0.95mL, 6.8 mmol) in dichloromethane (10 mL) at 0° C. under nitrogen. After 20 mins, the mixture was allowed to warm to room temperature and stirred for a further 30 mins. Water (10 mL) was added and the organic layer was separated and the solvent evaporated under reduced pressure. The residue was taken up in acetonitrile (10 mL) and 2-isopropylimidazole (250 mg, 2.27 mmol) and cesium carbonate (880 mg, 22.7 mmol) were added. The mixture was refluxed for 3 days and then allowed to cool. The mixture was filtered and the solvent was removed in vacuo. The residue was filtered through a silica plug using ethyl acetate and the solvent was removed under reduced pressure. The residue was dissolved in methanol (5 mL) and ethereal hydrogen chloride (5 mL of a 2N solution) was added. The mixture was stirred overnight, the solvent was removed in vacuo and the residue was chromatographed on silica gel using an elution gradient of dichloromethane to 94:6:0.6 dichloromethane:methanol:0.88 ammonia as eluant to afford the title compound as a white solid, 28 mg.

LRMS: m/z APCI 194 [MH]⁺

### Preparation 21

### 4-(2-Methyl-1H-imidazol-1-yl)piperidine

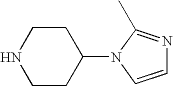

The procedure of Preparation 20 was followed reacting 2-methylimidazole with 1-tertbutyoxycarbonyl-4-methanesulphonyloxypiperidine to give the title compound as a white solid, 53 mg.

LRMS: m/z APCI 166 [MH]⁺

### Preparation 22

### 5-Methylhexane-2,4-dione

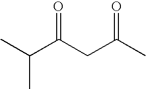

A mixture of 3-methyl-2-butanone (3.57 mL, 33.3 mmol) and ethyl acetate (9.8 mL, 99.9 mmol) in THF (30 mL) was added dropwise to a stirring solution of potassium tert-butoxide (50 mL of a 1N solution in THF, 50 mmol) in THF (100 mL) at 60° C. under nitrogen. The mixture was stirred at this temperature for 3 hours and then cooled to room temperature and stirred for a further 16 hours. Dilute hydrochloric acid (30 mL of a 2N solution) was added and the mixture was extracted with diethylether (3×20 mL). The combined organic fractions were washed with brine, dried (MgSO₄) and the solvent was removed in vacuo. The residue was chromatographed on silica gel using 98:2 pentane:ether as eluant to afford the title compound as a yellow oil, 2.83 g.

LRMS: m/z APCI 129 [MH]⁺

### Preparation 23

### 1-Benzyl-4-(5-isopropyl-3-methyl-1H-Pyrazol-1-yl)piperidine

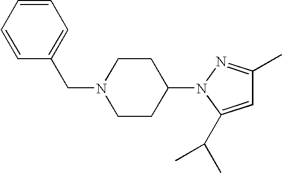

1-benzyl-4-hydrazinopiperidine (5.6 g, 27.3 mmol) (preparation 47) in ethanol (20 mL) was added dropwise to a stirring solution of the compound of Preparation 22 (2.33 g, 18.2 mmol) in ethanol (100 mL) at room temperature and the mixture was stirred for 16 hours. The solvent was removed in vacuo and the residue was chromatographed on silica gel using 98:2:0.2 dichloromethane:methanol:0.88 ammonia as eluant to give the title compound as a yellow oil, 1.4 g.

LRMS: m/z APCI-298 [MH]⁺

### Preparation 24

### 4-(5-isoPronyl-3-methyl-1H-pyrazol-1-yl)piperidine

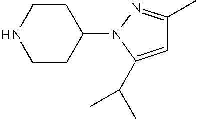

A mixture of the benzyl amine of Preparation 23 (1.4 g, 4.7 mmol), ammonium formate (1.48 g, 23.5 mmol) and palladium hydroxide (0.15 g) in ethanol (60 mL) was stirred at 50° C. for 4 hours. The cooled mixture was filtered through Arbocel® and the filtrate evaporated under reduced pressure to give a yellow oil, 1.03 g.

LRMS: m/z APCI-208 [MH]⁺

### Preparation 25

### N-(1-Benzylpiperidin-4-yl)-2-methylpropanamide

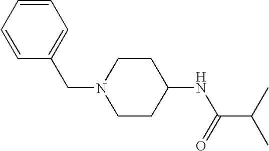

Isobutyryl chloride (0.33 mL, 3.16 mmol) was added dropwise to a stirring solution of 4-amino-1-benzylpiperidine (0.54 mL, 2.63 mmol) and triethylamine (0.44 mL, 3.16 mmol) in dichloromethane (5 mL) at 0° C. under nitrogen. After 6 hours, the reaction mixture was diluted with dichloromethane (15 mL), washed with saturated sodium hydrogen carbonate solution (15 mL), dried (MgSO₄) and the solvent removed in vacuo to give the title compound as a white solid, 673 mg.

LRMS: m/z APCI-261 [MH]⁺

### Preparation 26

### 1-Benzyl-4-(5-isopropyl-1H-tetrazol-1-yl)piperidine

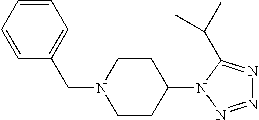

Phosphorus pentachloride (263 mg, 1.27 mmol) was added to a stirring solution of the compound of Preparation 25 (300 mg, 1.15 mmol) in dichloromethane (4 mL) at 5° C. under nitrogen. The mixture was allowed to warm to room temperature and stirred for 5 hours. The mixture was cooled to −5° C. and azido trimethylsilane (0.31 ml, 2.3 mmol) was added dropwise. The mixture was allowed to warm to room temperature and stirred for 16 hours. The mixture was cooled to 0° C. and saturated sodium hydrogen carbonate solution (5 mL) was added dropwise. The two layers were separated and the organic layer was washed with water (5 mL), brine, dried (MgSO₄) and the solvent removed under reduced pressure to give the title compound as a white solid, 275 mg (83%).

LRMS: m/z APCI-286 [MH]⁺

### Preparation 27

### 4-(5-isoPropyl-1H-tetrazol-1-yl)piperidine

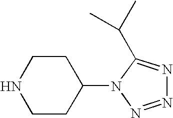

The procedure of Preparation 24 was followed reacting the compound of Preparation 26 with ammonium formate and palladium hydroxide to give the title compound as a white solid in 81% yield.

LRMS: m/z APCI 196 [MH]⁺

### Preparation 28

### 1-Benzylpiperidine-4-carboxylic acid

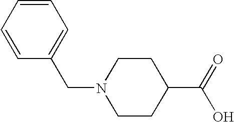

Sodium hydroxide solution (4.12 ml of a 1N solution, 4.12 mmol) was added to a stirring solution of ethyl 1-benzylpiperidine-4-carboxylate (1 g, 4.04 mmol) in ethanol (6 mL). After 24 hours, extra sodium hydroxide solution (4 mL) was added and the mixture was stirred for a further 24 hours. Dilute hydrochloric acid (4.06 ml of a 2N solution) was added to the mixture and the solvent was removed in vacuo. The residue was extracted with hot isopropyl alcohol (20 mL) and the solvent was evaporated in vacuo to low volume and allowed to stand at room temperature. The resulting solid was filtered off, washed with isopropyl alcohol and dried in vacuo to give the title compound as a white solid, 337 mg.

LRMS: m/z ES 220 [MH]⁺

### Preparation 29

### 1-Benzyl-N-isopropylpiperidine-4-carboxamide

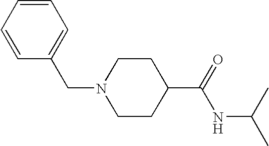

The acid of Preparation 28 (337 mg, 1.54 mmol) and isopropylamine (91 mg, 1.54 mmol) were dissolved in dichloromethane (10 mL) and N-ethyl-N′-(3-dimethylaminopropyl)carbodiimide methiodide (549 mg, 1.85 mmol) and 1-hydroxy benzotriazole monohydrate (10 mg, 0.077 mmol) were added. The mixture was stirred for 3 hours under nitrogen and then diluted with dichloromethane (10 mL) and washed with saturated sodium hydrogen carbonate solution (10 mL). The organic layer was washed with brine, dried (MgSO₄) and the solvent removed in vacuo to give the title compound as a white solid, 381 mg.

LRMS: m/z APCI-261 [MH]⁺

### Preparation 30

### 1-Benzyl-4-(1-isopropyl-1H-tetrazol-5-yl)piperidine

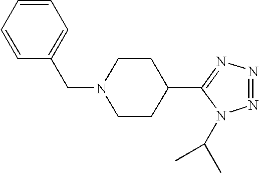

The procedure of Preparation 26 was followed reacting the compound of Preparation 29 with phosphorus pentachloride and subsequently with azido trimethylsilane to give the title compound as a white solid in 42% yield.

LRMS: m/z ES 286 [MH]⁺

### Preparation 31

### 4-(1-isoPropyl-1H-tetrazol-5-yl)piperidine

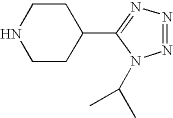

The procedure of Preparation 24 was followed reacting the compound of Preparation 30 with ammonium formate and palladium hydroxide to give the title compound as a white solid in quantitative yield.

LRMS: m/z ES 196 [MH]⁺

### Preparation 32

### 4-(1H-imidazol-1-yl)piperidine

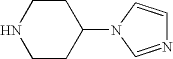

The procedure of Preparation 15 was followed reacting tert-butyl 4-(1H-imidazol-1-yl)piperidine-1-carboxylate (formed using process of preparation 20) with ethereal hydrogen chloride to give the title compound, the hydrochloride salt, as a white solid, 200 mg.

LRMS: m/z APCI 152 [MH]⁺

### Preparation 33

### 4-(3-isopropyl-1,2,4-oxadiazol-5-yl)piperidine

The above titled compound may be prepared as follows below. Reflux isobutyronitrile with hydroxylamine hydrochloride and sodium carbonate in methanol/water to form N-hydroxy-isobutyramide of general formula (XLVIII) in scheme 13. This compound can then be coupled piperidine 1,4-dicarboxylic acid mono-tert buytl ester (XLIX, scheme 13) using CDI in dichloromethane at room temperature to form the amide ester (representative of general formula XLX). This compound can then be refluxed with dioxane to form 4-(3-isopropyl-[1,2,4]oxadiazol-5-yl)piperidine (representative of general formula XLXI). The t-boc group can then be subsequently removed by adding hydrogen chloride in ether\methanol at room temperature to form the title compound.

### Preparation 34

### 4-(3-methyl-1,2,4-oxadiazol-5-yl)piperidine

The process of preparation 33 was followed but acetonitrile was substituted for isobutyronitrile.

### Preparation 35

### 4-(3-ethyl-1,2,4-oxadiazol-5-yl)piperidine

The process of preparation 33 was followed but propionnitrile was substituted for isobutyronitrile.

### Preparation 36

### 4-(3-secbutyl-1,24-oxadiazol-5-yl)piperidine

The process of preparation 33 was followed but secpentanyl nitrile precursor was substituted for isobutyronitrile.

### Preparation 37

### Ethyl (3S)-3-[(cyclobutylcarbonyl)amino]-3-phenylpropanoate

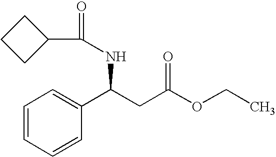

Cyclobutane carboxylic acid chloride (10 mL, 87.4 mmol) was added dropwise to an ice-cooled solution of ethyl (3S)-3-amino-3-phenylpropanoate (20 g, 87.4 mmol) (prepared in accordance with preparation 4 of WO 0039125) and Et₃N (30.5 mL, 219 mmol) in DCM (300 mL). Once addition was complete, the reaction was stirred at rt for 18 h. The mixture was diluted with water (200 mL) and the layers separated. The organic solution was washed with water (2×200 mL), dried (MgSO₄) and evaporated under reduced pressure to afford the title compound as a crystalline solid, 21.5 g.

LRMS: m/z APCI-276 [MH]⁺

### Preparation 38

### Ethyl (3S)-3-[(tert-butoxycarbonyl)amino]-3-Phenylpropanoate

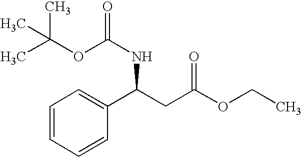

Et₃N (53.4 mL, 383 mmol) and di-tert-butyl dicarbonate (75.95 g, 348 mmol) were added to an ice-cooled solution of ethyl (3S)-3-amino-3-phenylpropanoate (80 g, 348 mmol) (refer to preparation 4 of WO 0039125) in THF (500 mL) and the mixture stirred at rt for 18 h. The mixture was diluted with EtOAc (500 mL), then washed with water (500 mL), 10% citric acid solution (3×500 mL), water (500 mL) and brine (500 mL). The organic solution was dried (MgSO₄) and evaporated under reduced pressure to afford the title compound as a white solid in quantitative yield.

LRMS: m/z APCI-294 [MH]⁺

### Preparation 39

### tert-Butyl {(1S)-3-[methoxy(methyl)amino]-3-oxo-1-phenylpropyl}carbamate

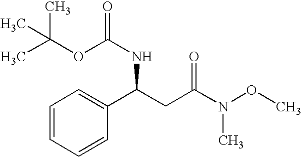

iso-Propylmagnesium chloride (250 ml of a 2N solution in diethyl ether, 500 mmol) was added dropwise to a solution of the ester from preparation 2 (47.74 g, 160 mmol) and N,O-dimethylhydroxylamine hydrochloride (24.19 g, 248 mmol) in THF (500 mL) at −10° C. under nitrogen, so as to maintain the temperature below −5° C. during the addition. The mixture was stirred for 1.5 h and then left at 4° C. for 18 h. Additional iso-propylmagnesium chloride (60 mL, 120 mmol) and N,O-dimethylhydroxylamine hydrochloride (7.80 g, 80 mmol) were added and the reaction stirred for a further 2 h at 0° C. The reaction was quenched by the careful addition of saturated ammonium chloride solution (200 ml), followed by water (200 mL). The layers were separated, the aqueous phase extracted with EtOAc (500 mL), and the organic solutions washed with water (250 mL) and brine (250 mL), dried (MgSO₄) and evaporated under reduced pressure. The residual oil was purified by column chromatography on silica gel using EtOAc:pentane (5:95) to afford the title compound as a golden oil, 26.8 g.

LRMS: m/z APCI-309 [MH]⁺

### Preparation 40

### tert-Butyl {(1S)-1-(3-fluorophenyl)-3-[methoxy(methyl)amino]-3-oxopropyl}carbamate

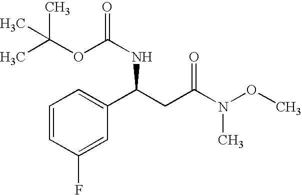

iso-Propylmagnesium chloride (242.1 mL of a 2N solution in diethyl ether, 484.2 mmol) was added dropwise to a stirred solution of methyl (3S)-3-[(tert-butoxycarbonyl)amino]-3-(3-fluorophenyl)propanoate (EP 1013276 preparation 30) (24 g, 80.7 mmol) and N,O-dimethylhydroxylamine hydrochloride (23.6 g, 242.2 mmol) in THF (240 mL) at −10° C. under nitrogen. The temperature was maintained below −5° C. during the addition. The mixture was stirred for 1.5 h and then left at 4° C. for 18 h. The mixture was cooled to −10° C. and saturated ammonium chloride solution (60 mL) was added. After stirring for 15 min, dilute hydrochloric acid (150 mL of a 2N solution) was added and the mixture was extracted with EtOAc (3×100 mL). The combined organic fractions were washed with brine, dried (MgSO₄) and the solvent removed under reduced pressure to give the desired compound as a yellow oil, 28 g.

LRMS: m/z APCI-327 [MH]⁺

### Preparation 41

### N-{(1S)-3-[Methoxy(methyl)amino]-3-oxo-1-phenylpropyl}cyclobutanecarboxamide

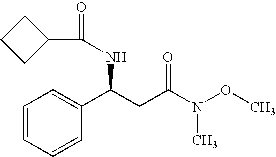

Isopropyl magnesium chloride (310 mL, 2M in THF, 620 mmol) was added dropwise to an ice-cooled solution of the ester from preparation 1 (21.5 g, 78 mmol) and N,O-dimethylhydroxylamine hydrochloride (30.5 g, 310 mmol) in THF (400 mL), so as to maintain the internal temperature below 4° C. Once addition was complete, the mixture was stirred at 0° C. for an hour. The reaction was quenched by the addition of ammonium chloride solution (400 mL) and the mixture extracted with EtOAc (300 mL). The organic extracts were dried (MgSO₄) and evaporated under reduced pressure to afford the title compound as a crystalline solid, 21.4 g.

LRMS: m/z APCI-291 [MH]⁺

### Preparation 42

### tert-Butyl [(1S)-3-oxo-1-phenylbutyl]carbamate

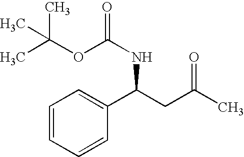

Methylmagnesium bromide (3M in ether, 90.7 mL, 272.1 mmol) was added dropwise to a stirred solution of the amide from preparation 3 (27.97 g, 90.7 mmol) in THF (250 mL) at −78° C. under nitrogen. The mixture was stirred at this temperature for 6 h and then left at 4° C. for 16 h. The mixture was cooled to −78° C. and saturated ammonium chloride solution (100 mL) was added dropwise. The mixture was then allowed to warm to rt and water (500 mL) was added. The mixture was extracted with EtOAc (3×400 mL) and the combined organic fractions were washed with water (500 mL) and brine (500 mL), dried (MgSO₄) and the solvent removed under reduced pressure to give the title compound as a solid, 21.58 g.

LRMS: m/z ES 264 [MH]⁺

### Preparation 43

### tert-Butyl [(1S)-1-(3-fluorophenyl)-3-oxobutyl]carbamate

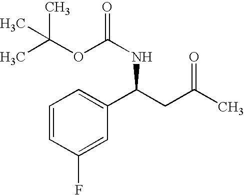

Methylmagnesium chloride (80.7 mL of a 3N solution in THF, 242.1 mmol) was added dropwise to a stirred solution of the amide from preparation 4 (26.3 g, 80.7 mmol) in THF (250 mL) at −78° C. under nitrogen. The mixture was stirred at this temperature for 2 h and then left at 4° C. for 16 h. The mixture was cooled to −78° C. and saturated ammonium chloride solution (15 mL) was added dropwise. The mixture was allowed to warm to rt and dilute hydrochloric acid (80 mL of a 2N solution) was added. The mixture was extracted with EtOAc (3×100 mL) and the combined organic fractions were washed with brine, dried (MgSO₄) and the solvent removed under reduced pressure to give the title compound as a white solid, 21.8 g.

LRMS: m/z APCI-282 [MH]⁺

### Preparation 44

### N-[(1S)-3-Oxo-1-phenylbutyl]cyclobutanecarboxamide

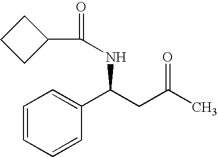

The title compound was obtained as a white foam in quantitative yield, from the compound of preparation 5, following the procedure described in preparation 7.

LRMS: m/z APCI-246 [MH]⁺

### Preparation 45

### tert-Butyl 2-(1-benzylpiperidin-4-ylidene)hydrazinecarboxylate

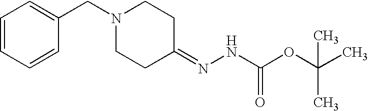

tert-Butyl carbazate (27.88 g, 211 mmol) and glacial acetic acid (26 mL) were added to solution of 1-benzyl-4-piperidone (40 g, 211 mmol) in DCM (400 mL) and the mixture stirred at rt for 18 h. The mixture was carefully neutralised by the addition of saturated sodium bicarbonate solution, and the layers separated. The organic phase was washed with further saturated sodium bicarbonate solution (200 mL), water (200 mL) and brine (200 mL). The solution was dried (MgSO₄) and evaporated under reduced pressure to provide the title compound in quantitative yield.

LRMS: m/z APCI-304 [MH]⁺

### Preparation 46

### tert-Butyl 2-(1-benzylpiperidin-4-yl)hydrazinecarboxylate

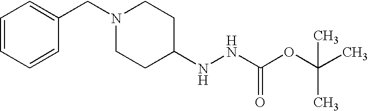

Sodium borohydride (23.95 g, 633 mmol) was added portionwise to an ice-cooled solution of the compound from preparation 11 (64.02 g, 211 mmol) in DCM (500 mL) and acetic acid (285 mL). The mixture was allowed to warm to rt and stirred for 24 h. The mixture was re-cooled in an ice-bath, carefully quenched with water, and the mixture concentrated under reduced pressure to remove the organic solvent. The solution was diluted with DCM (200 mL) and basified using solid sodium hydroxide. The layers were separated, the aqueous phase extracted with DCM (2×200 mL) and the combined organic solutions washed with water (300 mL), dried (MgSO₄) and evaporated under reduced pressure to afford the crude title compound, 63.0 g.

LRMS: m/z APCI-306 [MH]⁺

### Preparation 47

### 1-Benzyl-4-hydrazinopiperidine dihydrochloride

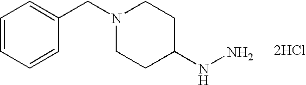

HCl gas was bubbled through an ice-cooled solution of the compound from preparation 12 (63.0 g, 206 mmol) in MeOH (500 mL), and once saturated the solution was stirred at rt for 72 h. The solution was re-saturated with HCl gas periodically, and the mixture allowed to stir at rt for a further 36 h. The solution was evaporated under reduced pressure to afford the crude title compound as a white solid.

LRMS: m/z APCI-206 [MH]⁺

### Preparation 48

### 1-Benzyl-4-(3-isopropyl-5-methyl-4H-1,2,4-triazol-4-yl)piperidine

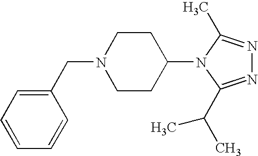

Phosphorous pentachloride (59.8 g, 287 mmol) was added portionwise over 20 min to an ice-cooled solution of the compound from preparation 9 (57.8 g, 220 mmol) in DCM (500 mL), the solution was allowed to warm to rt and stirred for 2 h. The solution was re-cooled in an ice-bath, a slurry of acetic hydrazide (48.94 g, 661 mmol) in tert-amyl alcohol (300 mL) added and the mixture allowed to stir at rt for 16 h. The solution was concentrated under reduced pressure and the residue re-suspended in toluene (250 mL) and dioxan (250 mL) and 4-toluenesulphonic acid (1.4 g, 7.36 mmol) added. The mixture was heated under reflux for 5 h, then allowed to cool. The mixture was concentrated under reduced pressure, the residue partitioned between water (400 mL) and DCM (400 mL) and the layers separated. The aqueous layer was washed with DCM (10 mL) then basified to pH 8 using solid potassium hydroxide. The aqueous solution was extracted with DCM (1 L in total), the organic extracts dried (MgSO₄) and evaporated under reduced pressure. The residual oil was crystallised from diethyl ether to afford the title compound as a white solid, 10.4 g.

The ethereal solution was concentrated under reduced pressure and the residue purified by column chromatography on silica gel using an elution gradient of EtOAc:MeOH (100:0 to 80:20) to afford additional compound, 28.6 g.

LRMS: m/z APCI-299 [MH]⁺

### EXAMPLE 1

### Methyl 3-(1-{(3S)-3-[(tert-butoxycarbonyl)amino]-1-methyl-3-phenylpropyl}piperidin-4-yl)-2-methyl-3,4,6,7-tetrahydro-5H-imidazo[4,5-c]pyridine-5-carboxylate

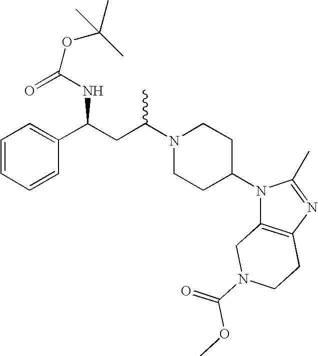

tert-Butyl [(1S)-3-oxo-1-phenylbutyl]carbamate (3.73 g, 14.2 mmol) (preparation 42) and the compound of Preparation 6 (5.12 g, 18.4 mmol) were dissolved in ethanol (70 mL) under nitrogen and titanium tetraisopropoxide (20.9 mL, 70.8 mmol) was added. The mixture was stirred for 24 hours and then sodium cyanoborohydride (1.33 g, 21.2 mmol) was added. The mixture was stirred for 3 days. Saturated sodium hydrogen carbonate solution (150 mL) was added and after 15 mins the mixture was diluted with ethyl acetate (150 mL) and filtered through Celite®. The resulting solution was washed with water (150 mL), brine, dried (MgSO₄) and the solvent was evaporated under reduced pressure. The residue was purified by column chromatography on silica gel, eluting with dichloromethane:methanol:0.88 ammonia (97.5:2.5:0.25) to afford a white solid, 3.89 g (52%).

LRMS: m/z APCI-526 [MH]⁺

### EXAMPLES 2 AND 3

### Methyl 3-(1-{(3S)-3-[(tert-butoxycarbonyl)amino]-1-methyl-3-phenylpropyl}piperidin-4-yl)-2-methyl-3,4,6,7-tetrahydro-5H-imidazo[4,5-c]pyridine-5-carboxylate

The compound of Example 1 (2.23 g) was purified by column chromatography on silica gel, eluting with 99:1:0.1 dichloromethane:methanol:0.88 ammonia increasing to 97:3:0.3 dichloromethane:methanol:0.88 ammonia. 741 mg of the first eluting diastereoisomer (Rf=0.15 in 95:5:0.5 dichloromethane:methanol:0.88 ammonia) and 857 mg of the second eluting diastereoisomer (Rf=0.125 in 95:5:0.5 dichloromethane:methanol:0.88 ammonia) were isolated.

Example 2: For Diastereoisomer 1: LRMS: m/z APCI-526 [MH]⁺; ¹H-NMR (CD₃OD, 400 MHz): δ 7.36-7.28 (4H, m), 7.22 (1H, m), 4.67-4.63 (2H, m), 3.94 (1H, m), 3.76-3.63 (2H, m), 3.73 (3H, s), 2.90 (1H, m), 2.80 (1H, m), 2.68-2.50 (4H, m), 2.38 (3H, s), 2.33 (1H, m), 2.15-1.82 (5H, m), 1.66 (1H, m), 1.41 (9H, s), 1.32 (1H, m), 1.01 (3H, d); LRMS: m/z APCI 526 [MH]⁺

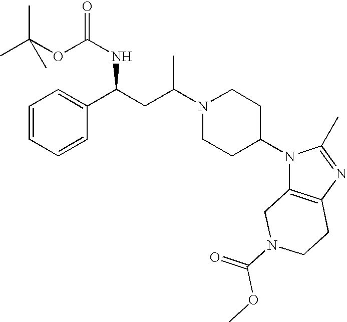

Example 3: For Diastereoisomer 2: LRMS: m/z APCI-526 [MH]⁺; ¹H-NMR (CD₃CD, 400 MHz): δ 7.36-7.28 (4H, m), 7.22 (1H, m), 4.72-4.61 (2H, m), 3.97 (1H, m), 3.78-3.63 (2H, m), 3.74 (3H, s), 2.99 (1H, m), 2.77 (1H, m), 2.62 (1H, m), 2.61-2.46 (3H, m), 2.41 (1H, m), 2.38 (3H, s), 2.14-1.82 (5H, m), 1.61 (1H, m), 1.40 (9H, s), 1.32 (1H, m), 1.04 (3H, d);

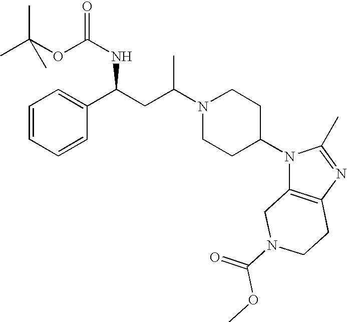

### EXAMPLE 4

### Methyl 3-{1-[(3S)-3-amino-1-methyl-3-phenylpropyl]piperidin-4-yl}-2-methyl-3,4,6,7-tetrahydro-5H imidazo[4,5-c]pyridine-5-carboxylate

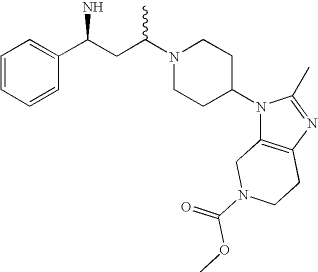

The compound of Example 1 (3.89 g, 7.40 mmol) was dissolved in ethyl acetate (150 mL) and 2N ethereal hydrochloric acid (20 mL) was added. The mixture was stirred at room temperature for 16 hours and the mixture was concentrated in vacuo to give the title compound as a white solid, 4.2 g.

LRMS: m/z APCI-426 [MH]⁺

### EXAMPLES 5 TO 8

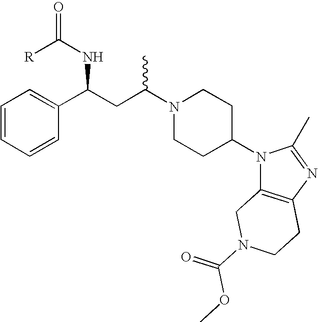

The compound of Example 4 (1 eq.) and the appropriate acid (1.05 eq.) were dissolved in Dimethylformamide (20 mLmmol⁻¹) at room temperature. 1-hydroxy benzotriazole monohydrate (1.2 eq.) and 1-(3-dimethylamino-3-ethylcarbodiimide hydrochloride (1.2 eq.) were added to the mixture followed by N,N-diisopropylethylamine (5 eq.) and the reaction was stirred at room temperature under nitrogen for 24-48 hours. The solvent was removed under reduced pressure and the residue was dissolved in dichloromethane (3 mL), washed with saturated sodium hydrogen carbonate (3 mL) and the solvent removed in vacuo. The residue was purified by column chromatography on silica gel using dichloromethane:methanol:0.88 ammonia as eluant.

| Ex. No. | Data |
|---|---|
| 5 | R = 2,2-difluorocyclobutyl; |
| | LRMS: APCI m/z 544 [MH]⁺ |
| 6 | R = 4,4-difluorocyclohexyl; |
| | LRMS: APCI m/z 572 [MH]⁺ |
| 7 | R = cyclopropylmethyl; |
| | LRMS: APCI m/z 508 [MH]⁺ |
| 8 | R = 1,1-dioxo-tetrahydrothiopyran-4-yl |
| | (Org. Preperations and Procedures |
| | International, 1977, 9, 94-6) |
| | LRMS: APCI m/z 586 [MH]⁺ |

2,2-Difluorocyclobutylcarboxylic acid was obtained as described in Syn. Comm., 2005, 35, 657-662

4,4-Difluorocyclohexylcarboxylic acid was obtained as described in WO 9727185, preparation 9(a).

### EXAMPLES 9 TO 14

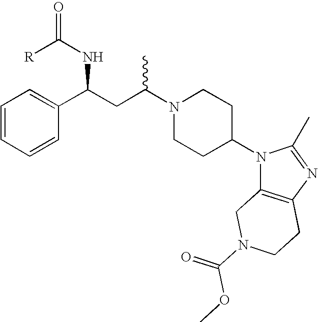

The appropriate acid chloride (1.05 eq) was added to a solution of example 4 (1 eq.) and triethylamine (5 eq.) in N,N-dimethylformamide (10 mLmmol⁻¹), and the reaction stirred at room temperature for 18 hours. The reaction was concentrated under reduced pressure, the residue partitioned between dichloromethane and 10% sodium bicarbonate solution and this mixture shaken for 30 minutes. The layers were separated, the aqueous solution extracted with dichloromethane and the combined organic extracts evaporated under reduced pressure to afford the title compound.

| Ex. No. | Data |
|---|---|
| 9A | R = methyl; |
| 9B | Diastereoisomers separated; |
| | LRMS: APCI m/z 508 [MH]⁺ for both. |
| 10 | R = cyclobutyl; |
| | LRMS: APCI m/z 508 [MH]⁺ |
| 11 | R = isopropyl; |
| | LRMS: APCI m/z 496 [MH]⁺ |
| 12 | R = cyclopropyl; |
| | LRMS: m/z 494 [MH]⁺ |
| 13A | R = methoxy; |
| 13B | Diastereoisomers separated; |
| | LRMS: APCI m/z 484 [MH]⁺ for both. |
| 14 | R = isopropoxy; LRMS: APCI m\z 512 [MH]⁺ |

### EXAMPLE 15

tent-Butyl {(1S)-3-[4-(2-methyl-4,5,6,7-tetrahydro-3H-imidazo[4,5-c]pyridin-3-yl)piperidin-1-yl]-1-phenylbutyl}carbamate

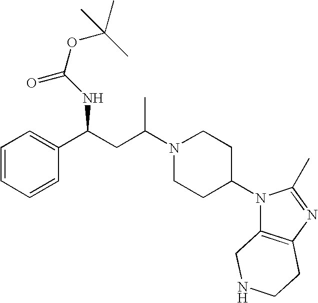

The compound of Example 2 (730 mg, 1.39 mmol) and sodium hydroxide (8 mL of a 2N solution, 16 mmol) were heated to reflux in isopropanol (6 mL) for 48 hours. The mixture was cooled and the two layers were separated. The aqueous layer was extracted with isopropanol (6 mL) and the combined isopropanol layers were evaporated under reduced pressure. The residue was purified by column chromatography on silica gel using 92:8:0.8 dichloromethane:methanol:0.88 ammonia as eluant to afford the title compound as a white solid, 467 mg.

LRMS: m/z ES 468 [MH]⁺

### EXAMPLE 16

### tert-Butyl {(1S)-3-[4-(2-methyl-4,5,6,7-tetrahydro-3H-imidazo[4,5-c]pyridin-3-yl)piperidin-1-yl]-1-phenylbutyl}carbamate

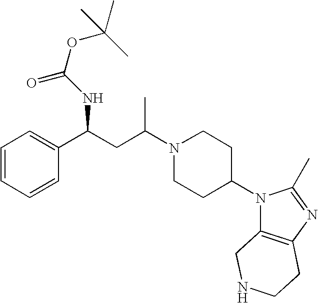

The compound of Example 3 (1.07 g, 2.03 mmol) and sodium hydroxide (11 mL of a 2N solution, 22 mmol) were heated to reflux in isopropanol (9 mL) for 48 hours. The mixture was cooled and the two layers were separated. The aqueous layer was extracted with isopropanol (6 mL) and the combined isopropanol layers were evaporated under reduced pressure. The residue was purified by column chromatography on silica gel using 92:8:0.8 dichloromethane:methanol:0.88 ammonia as eluant to afford the title compound as a white solid, 705 mg.

LRMS: m/z ES 468 [MH]⁺

### EXAMPLE 17

### tert-Butyl {(1S)-3-[4-(5-isobutyryl-2-methyl-4,5,6,7-tetrahydro-3H-imidazo[4,5-c]pyridin-3-yl)piperidin-1-yl]-1-phenylbutyl}carbamate

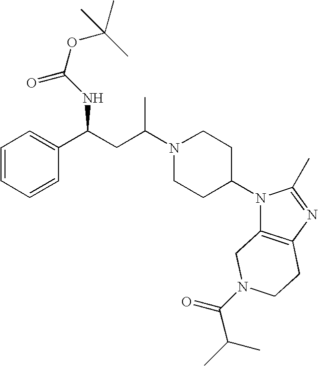

Isobutyryl chloride (0.13 mL, 1.2 mmol) was added to a stirring mixture of the compound of Example 15 (467 mg, 1 mmol) and potassium carbonate (166 mg, 1.2 mmol) in isopropanol (5 mL) at room temperature. After 3 hours, the reaction mixture was filtered and the solvent was removed in vacuo. The residue was purified by column chromatography on silica gel using ethyl acetate:methanol:0.88 ammonia (95:5:0.5) as eluant to afford the title compound as a white solid, 469 mg.

LRMS: m/z ES 538 [MH]⁺

### EXAMPLE 18

### tert-Butyl {(1S)-3-[4-(5-isobutyryl-2-methyl-4,5,6,7-tetrahydro-3H-imidazo[4,5-c]pyridin-3-yl)piperidin-1-yl]-1-phenylbutyl}carbamate

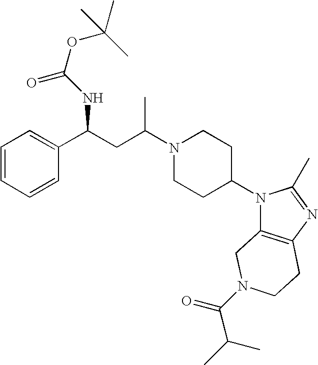

Isobutyryl chloride (0.14 mL, 1.3 mmol) was added to a stirring mixture of the compound of Example 16 (505 mg, 1.08 mmol) and potassium carbonate (179 mg, 1.3 mmol) in isopropanol (5 mL) at room temperature. After 3 hours, the reaction mixture was filtered and the solvent was removed in vacuo. The residue was purified by column chromatography on silica gel using ethyl acetate:methanol:0.88:ammonia (95:5:0.5) as eluant to afford the title compound as a white solid, 525 mg.

LRMS: m/z ES 538 [MH]⁺

### EXAMPLE 19

### (1S)-3-[4-(5-isoButyryl-2-methyl-4,5,6,7-tetrahydro-3H-imidazo[4,5-c]pyridin-3-yl)piperidin-1-yl]-1-phenylbutan-1-amine

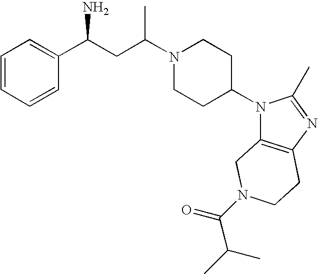

The compound of Example 17 (469 mg, 0.87 mmol) was dissolved in ethyl acetate (25 mL) and the mixture was cooled to 0° C. Hydrogen chloride gas was bubbled through the mixture for 20 minutes and then it was stirred for a further 1 hour. The mixture was evaporated under reduced pressure and triturated with pentane to give a white solid, 438 mg.

LRMS: m/z ES 438 [MH]⁺

### EXAMPLE 20

### (1S)-3-[4-(5-isoButyryl-2-methyl-4,5,6,7-tetrahydro-3H-imidazo[4,5-c]pyridin-3-yl)piperidin-1-yl]-1-phenylbutan-1-amine

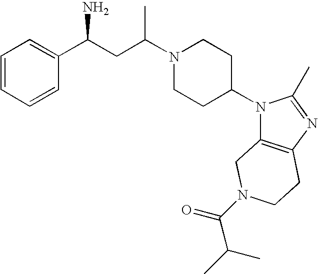

The compound of Example 18 (610 mg, 1.39 mmol) was dissolved in ethyl acetate (25 mL) and the mixture was cooled to 0° C. Hydrogen chloride gas was bubbled through the mixture for 20 minutes and then it was stirred for a further 1 hour. The mixture was evaporated under reduced pressure and triturated with pentane to give a white solid, 554 mg.

LRMS: m/z ES 438 [MH]⁺

### EXAMPLES 21 AND 22

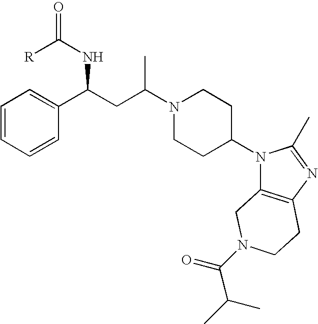

The free base of the compound of Example 19 (1 eq.) and the appropriate acid (1.1 eq.) were dissolved in dichloromethane (29 mLmmol⁻¹) at room temperature. 1-hydroxy benzotriazole monohydrate (0.05 eq.) and N-ethyl-N′-(3-dimethylaminopropyl)carbodiimide methiodide (1.2 eq.) were added to the mixture and the reaction was stirred at room temperature under nitrogen for 24-48 hours. Saturated sodium hydrogen carbonate (3 mL) was added to the mixture and the organic layer was evaporated under reduced pressure. The residue was purified by column chromatography on silica gel using dichloromethane:methanol:0.88 ammonia as eluant.

| Ex. No. | Data |
|---|---|
| 21 | R = 4,4-difluorocyclohexyl; |
| | LRMS: m/z ES 584 [MH]⁺ |
| 22 | R = Methylcyclopropyl; |
| | LRMS: m/z ES 542 [MNa]⁺ |

### EXAMPLES 23 AND 24

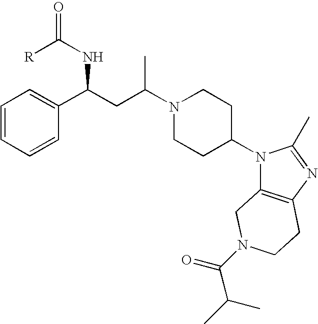

The appropriate acid chloride (1.06 eq) was added to a solution of the free base of the compound of Example 19 (1 eq.) and triethylamine (1.2 eq.) in N,N-dimethylformamide (10 mLmmol⁻¹), and the reaction stirred at room temperature under nitrogen for 24-48 hours. The reaction was concentrated under reduced pressure and the residue partitioned between dichloromethane and saturated sodium hydrogen carbonate solution. The layers were separated, the aqueous solution extracted with dichloromethane and the combined organic extracts evaporated under reduced pressure. The residue was purified by column chromatography on silica gel using dichloromethane:methanol:0.88 ammonia as eluant.

| Ex. No. | Data |
|---|---|
| 23 | R = isopropyl; |
| | LRMS: m/z ES 508 [MH]⁺; 530 [MNa]⁺ |
| 24 | R = cyclobutyl; |
| | LRMS: m/z ES 542 [MNa]⁺ |

### EXAMPLE 25

### 3,3-Difluoro-N-{(1S)-3-[4-(5-isobutyryl-2-methyl-4,5,6,7-tetrahydro-3H-imidazo[4,5-c]pyridin-3-yl)piperidin-1-yl]-1-phenylbutyl}cyclobutanecarboxamide

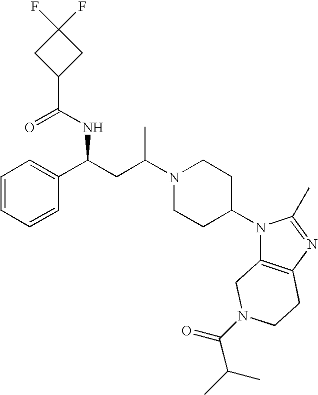

The free base of the compound of Example 19 (32 mg, 0.07 mmol) and 2,2-difluorocyclobutanecarboxylic acid (16 mg, 0.12 mmol) were dissolved in dichloromethane (2 mL) and polymer bound N-benzyl-N′-cyclohexylcarbodiimide (97 mg, 0.13 mmol) was added. The mixture was shaken for 3 days and then additional polymer bound N-benzyl-N′-cyclohexylcarbodiimide (200 mg) was added. After shaking for a further 24 hours, the resin was filtered off and the filtrate was washed with saturated sodium hydrogen carbonate. The organic layer was chromatographed through a silica plug using 95:5:0.5 dichloromethane:methanol:0.88 ammonia as eluent to give the title compound as a white solid, 28 mg.

LRMS: m/z ES 556 [MH]⁺

### EXAMPLES 26 TO 28

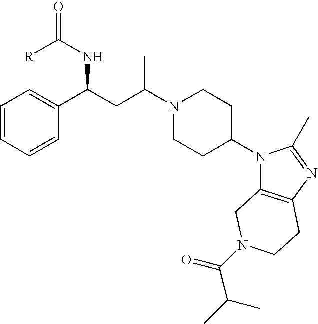

The free base of the compound of Example 20 (1 eq.) and the appropriate acid (1.1 eq.) were dissolved in dichloromethane (18 mLmmol⁻¹) at room temperature. 1-hydroxy benzotriazole monohydrate (0.05 eq.) and N-ethyl-N′-(3-dimethylaminopropyl)carbodiimide methiodide (1.2 eq.) were added to the mixture and the reaction was stirred at room temperature under nitrogen for 24-48 hours. Saturated sodium hydrogen carbonate (3 mL) was added to the mixture and the organic layer was evaporated under reduced pressure. The residue was purified by column chromatography on silica gel using dichloromethane:methanol:0.88 ammonia as eluant.

| Ex. No. | Data |
|---|---|
| 26 | R = 2,2-difluorocyclobutyl; |
| | LRMS: m/z ES 556 [MH]⁺; 578 [MNa]⁺ |
| 27 | R = methylcyclopropyl; |
| | LRMS: m/z ES 520 [MH]⁺; 542 [MNa]⁺ |
| 28 | R = 2-methylpropyl; |
| | LRMS: m/z ES 522 [MH]⁺; 544 [MNa]⁺ |

### EXAMPLES 29 TO 31

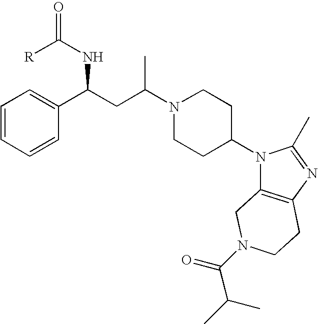

The appropriate acid chloride (1.06 eq) was added to a solution of the free base of the compound of Example 20 (1 eq.) and triethylamine (1.2 eq.) in N,N-dimethylformamide (10 mLmmol⁻¹), and the reaction stirred at room temperature under nitrogen for 24-48 hours. The reaction was concentrated under reduced pressure and the residue partitioned between dichloromethane and saturated sodium hydrogen carbonate solution. The layers were separated, the aqueous solution extracted with dichloromethane and the combined organic extracts evaporated under reduced pressure. The residue was purified by column chromatography on silica gel using dichloromethane:methanol:0.88 ammonia as eluant

| Ex. No. | Data |
|---|---|
| 29 | R = isopropyl; |
| | LRMS: m/z ES 508 [MH]⁺; 530 [MNa]⁺ |
| 30 | R = cyclobutyl; |
| | LRMS: m/z ES 520 [MH]⁺; 542 [MNa]⁺ |
| 31 | R = methyl; |
| | LRMS: m/z ES 480 [MH]⁺; 502 [MNa]⁺ |

### EXAMPLES 32 AND 33

### Methyl 3-{1-[(3S)-3-[(tert-butoxycarbonyl)amino]-3-(3-fluorophenyl)-1-methylpropyl]piperidin-4-yl}-2-methyl-3,4,6,7-tetrahydro-5H-imidazo[4,5-c]pyridine-5-carboxylate

tert-Butyl [(1S)-1-(3-fluorophenyl)-3-oxobutyl]carbamate (5.77 g, 20.5 mmol) (preparation 43) and the compound of Preparation 6 (7.42 g, 26.7 mmol) were dissolved in ethanol (50 mL) under nitrogen and titanium tetraisopropoxide (30.3 mL, 102.5 mmol) was added. The mixture was stirred for 3 days and then sodium cyanoborohydride (1.93 g, 30.75 mmol) was added. The mixture was stirred for 16 hours. Saturated sodium hydrogen carbonate solution (150 mL) was added and after 15 mins the mixture was diluted with dichloromethane (250 mL) and filtered through Celite®. The resulting solution was washed with water (150 mL), brine, dried (MgSO₄) and the solvent was evaporated under reduced pressure. The residue was purified several times by column chromatography on silica gel, eluting with dichloromethane:methanol:0.88 ammonia (99:1:0.1 to 98:2:0.2) to afford the two diastereoisomers.

### EXAMPLE 32

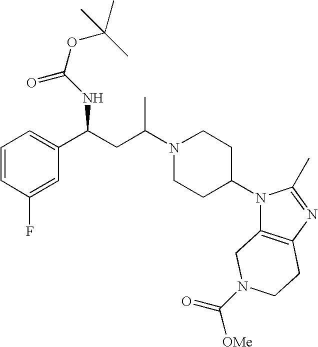

1.38 g of the compound of Example 32 (Rf=0.18 in 95:5:0.5 dichloromethane/methanol/0.88 ammonia)

### EXAMPLE 33

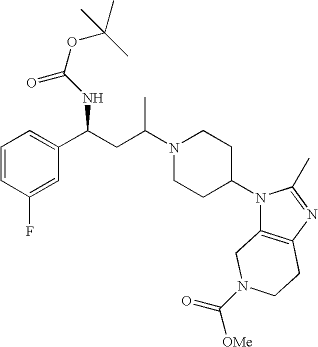

2.83 g of the compound of Example 33 (Rf=0.17 in 95:5:0.5 dichloromethane/methanol/0.88 ammonia) were isolated.

| Ex. No. | Data |
|---|---|
| 32 | LRMS: m/z ES 544 [MH]⁺ |
| | ¹H-NMR (CD₃OD, 400 MHz): δ 7.34 (1H, dt), |
| | 7.13 (1H, d), 7.05 (1H, m), 6.96 (1H, td), |
| | 4.66 (2H, m), 3.95 (1H, m), 378-3.62 (2H, m), |
| | 3.73 (3H, s), 2.92 (1H, m), 2.80 (1H, m), |
| | 2.74-2.49 (4H, m), 2.38 (3H, s), 2.33 (1H, td), |
| | 2.17-1.84 (5H, m), 1.65 (1H, m), 1.42 (9H, s), |
| | 1.34 (1H, m), 1.02 (3H, d). |
| 33 | LRMS: m/z ES 544 [MH]⁺ |
| | ¹H-NMR (CD₃OD, 400 MHz): δ 7.32 (1H, m), |
| | 7.16 (1H, d), 7.08 (1H, m), 6.95 (1H, td), |
| | 4.69 (2H, m), 3.97 (1H, m), 3.78-3.61 (2H, m), |
| | 3.74 (3H, s), 2.98 (1H, m), 2.75 (1H, m), |
| | 2.67-2.47 (4H, m), 2.39 (1H, m), 2.38 (3H, s), |
| | 2.13-1.84 (5H, m), 1.61 (1H, m), 1.40 (9H, s), |
| | 1.35 (1H, m), 1.04 (3H, d). |

### EXAMPLE 34

### tert-Butyl {(1S)-1-(3-fluorophenyl)-3-[4-(2-methyl-4,5,6,7-tetrahydro-3H-imidazo[4,5-c]pyridin-3-yl)piperidin-1-yl]butyl}carbamate

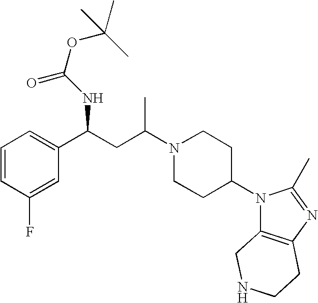

The compound of Example 32 (1.38 g, 2.5 mmol) and sodium hydroxide (13.6 mL of a 2N solution, 27 mmol) were heated to reflux in isopropanol (11 mL) for 24 hours. The mixture was cooled and the two layers were separated. The aqueous layer was extracted with isopropanol (20 mL) and the combined isopropanol layers were evaporated under reduced pressure. The residue was purified by column chromatography on silica gel using 95:5:0.5 dichloromethane:methanol:0.88 ammonia as eluant to afford the title compound as a white solid, 839 mg.

LRMS: m/z ES 486 [MH]⁺

### EXAMPLE 35

### tert-Butyl {(1S)-1-(3-fluorophenyl)-3-[4-(2-methyl-4,5,6,7-tetrahydro-3H-imidazo[4,5-c]pyridin-3-yl)piperidin-1-yl]butyl}carbamate

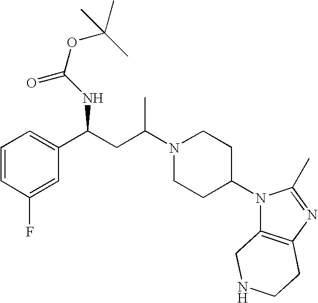

The compound of Example 33 (2.83 g, 5.2 mmol) and sodium hydroxide (28 mL of a 2N solution, 56 mmol) were heated to reflux in isopropanol (23 mL) for 24 hours. The mixture was cooled and the two layers were separated. The aqueous layer was extracted with isopropanol (30 mL) and the combined isopropanol layers were evaporated under reduced pressure. The residue was purified by column chromatography on silica gel using 95:5:0.5 dichloromethane:methanol: 0.88 ammonia as eluant to afford the title compound as a white solid, 1.98 g.

HRMS: Found. 486.3233

### EXAMPLE 36

### tert-Butyl[(1S)-3-[4-(5-acetyl-2-methyl-4,5,6,7-tetrahydro-3H-imidazo[4,5-c]pyridin-3-yl)piperidin-1-yl]-1-(3-fluorophenyl)butyl]carbamate

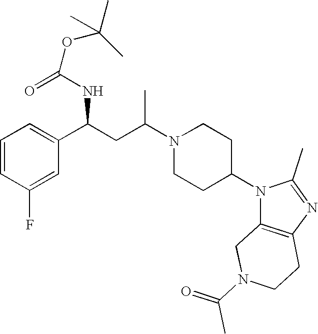

The compound of Example 34 (614 mg, 1.26 mmol) was dissolved in isopropanol (6 mL) and potassium carbonate (209 mg, 1.51 mmol) was added followed by acetyl chloride (0.11 mL, 1.51 mmol). The reaction was stirred at room temperature under nitrogen for 16 hours. Further potassium carbonate (174 mg) and acetyl chloride (0.09 mL) were added and the mixture was stirred for a further 24 hours. The reaction mixture was filtered and the filtrate was concentrated in vacuo. The residue was dissolved in ethyl acetate (10 mL) and washed with water (10 mL), saturated sodium hydrogen carbonate (10 mL) and brine (10 mL). The organic layer was dried (MgSO₄) and the solvent removed under reduced pressure to give a yellow solid, 616 mg.

LRMS: m/z ES 528 [MH]⁺

### EXAMPLE 37

### tert-Butyl [(1S)-3-[4-(5-acetyl-2-methyl-4,5,6,7-tetrahydro-3H-imidazo[4,5-c]pyridin-3-yl)piperidin-1-yl]-1-(3-fluorophenyl)butyl]carbamate

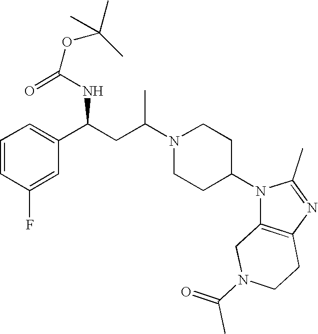

The procedure of Example 36 was followed reacting the compound of Example 35 with acetyl chloride to give the title compound as a yellow solid in 97% yield.

LRMS: m/z ES 528 [MH]⁺

### EXAMPLE 38

### tert-Butyl {(1S)-1-(3-fluorophenyl)-3-[4-(5-isobutyryl-2-methyl-4,5,6,7-tetrahydro-3H-imidazo[4,5-c]pyridin-3-yl)piperidin-1-yl]butyl}carbamate

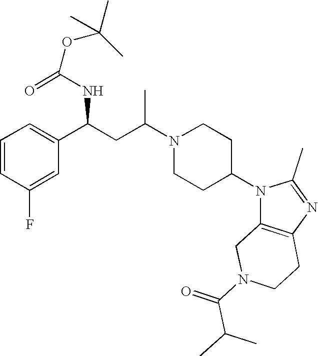

The procedure of Example 17 was followed reacting the compound of Example 34 with isobutyryl chloride to give the title compound as a yellow solid in 84% yield.

LRMS: m/z ES 556 [MH]⁺

### EXAMPLE 39

### tert-Butyl {(1S)-1-(3-fluorophenyl)-3-[4-(5-isobutyryl-2-methyl-4,5,6,7-tetrahydro-3H-imidazo[4,5-c]pyridin-3-yl)piperidin-1-yl]butyl}carbamate

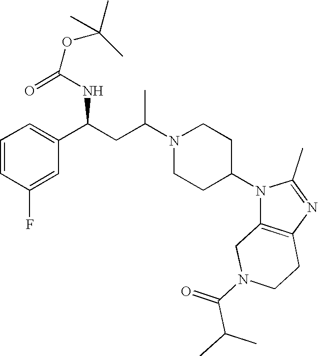

The procedure of Example 17 was followed reacting the compound of Example 35 with isobutyryl chloride to give the compound of example 39 as a yellow solid in 75% yield.

LRMS: m/z ES 556 [MH]⁺

### EXAMPLE 40

### Ethyl 3-{1-[(3S)-3-[(tert-butoxycarbonyl)amino]-3-(3-fluorophenyl)-1-methylpropyl]piperidin-4-yl}-2-methyl-3,4,6,7-tetrahydro-5H-imidazo[4,5-c]pyridine-5-carboxylate

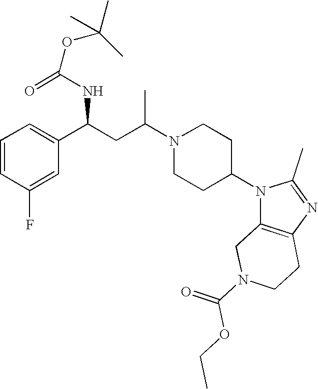

Ethyl chloroformate (0.016 mL, 0.161 mmol) was added to a stirring solution of the compound of Example 34 (65 mg, 0.134 mmol) and triethylamine (0.023 mL, 0.161 mmol) in dichloromethane (1 mL) under nitrogen. The reaction was stirred for 5 hours and then diluted with dichloromethane (10 mL) and washed with water (5 mL), saturated sodium hydrogen carbonate (5 mL), brine (5 mL) and dried (MgSO₄). The solvent was removed in vacuo and the residue was triturated with pentane to give a yellow solid, 58 mg (77%).

LRMS: m/z ES 558 [MH]⁺

### EXAMPLE 41

### Ethyl 3-{1-[(3S)-3-[(tert-butoxycarbonyl)amino]-3-(3-fluorophenyl)-1-methylpropyl]piperidin-4-yl}-2-methyl-3,4,6,7-tetrahydro-5H-imidazo[4,5-c]pyridine-5-carboxylate

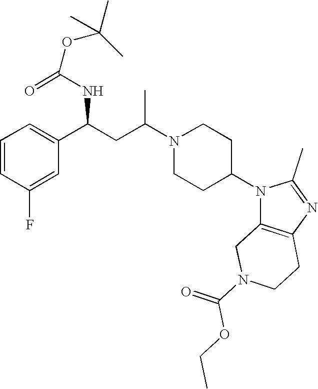

The procedure of Example 40 was followed reacting the compound of Example 35 with ethyl chloroformate to give the title compound as a yellow solid in 78% yield.

LRMS: m/z ES 558 [MH]⁺

### EXAMPLE 42

### (1S)-3-[4-(5-Acetyl-2-methyl-4,5,6,7-tetrahydro-3H-imidazo[4,5-c]pyridin-3-yl)piperidin-1-yl]-1-(3-fluorophenyl)butan-1-amine

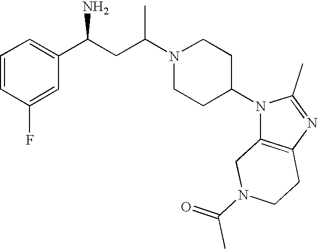

The procedure of Example 19 was followed reacting the compound of Example 36 with hydrogen chloride gas to give the title compound as a white solid in 92% yield.

LRMS: m/z ES 428 [MH]⁺

### EXAMPLE 43

### (1S)-3-[4-(5-Acetyl-2-methyl-4,5,6,7-tetrahydro-3H-imidazo[4,5-c]pyridin-3-yl)piperidin-1-yl]-1-phenylbutan-1-amine

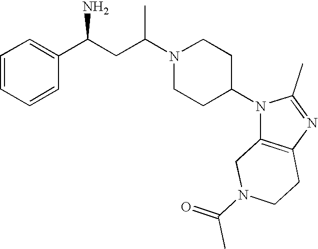

The procedure of Example 19 was followed reacting the compound of Example 132 with hydrogen chloride gas to give the title compound as a white solid in 92% yield.

LRMS: m/z ES 410 [MH]⁺

### EXAMPLE 44

### (1S)-1-(3-Fluorophenyl)-3-[4-(5-isobutyryl-2-methyl-4,5,6,7-tetrahydro-3H-imidazo[4,5-c]pyridin-3-yl)piperidin-1-yl]butan-1-amine

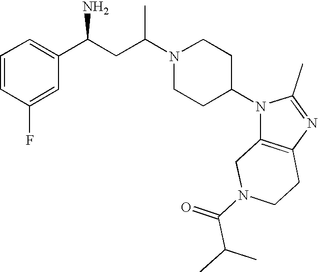

The procedure of Example 19 was followed reacting the compound of Example 38 with hydrogen chloride gas to give the title compound as a white solid in 80% yield.

LRMS: m/z ES 456 [MH]⁺

### EXAMPLE 45

### Ethyl 3-{1-[(3S)-3-amino-3-(3-fluorophenyl)-1-methylpropyl]piperidin-4-yl}-2-methyl-3,4,6,7-tetrahydro-5H-imidazo[4,5-c]pyridine-5-carboxylate

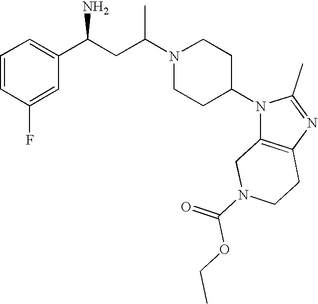

The procedure of Example 19 was followed reacting the compound of Example 40 with hydrogen chloride gas to give the title compound as a white solid in 55% yield.

LRMS: m/z ES 458 [MH]⁺

### EXAMPLE 46

### Methyl 3-{1-[(3S)-3-amino-3-(3-fluorophenyl)-1-methylpropyl]piperidin-4-yl}-2-methyl-3,4,6,7-tetrahydro-5H-imidazo[4,5-c]pyridine-5-carboxylate

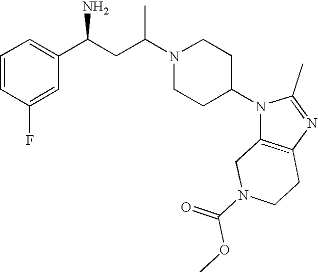

The procedure of Example 19 was followed reacting the compound of Example 32 with hydrogen chloride gas to give the title compound as a white solid in 93% yield.

LRMS: m/z ES 444 [MH]⁺

### EXAMPLES 47-56

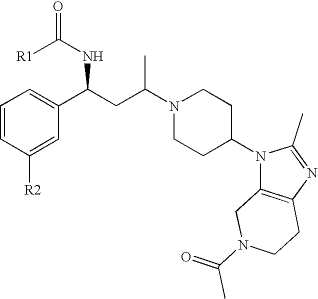

The procedure of Example 21 was followed reacting the compound of Example 42 or the compound of Example 43 with a set of acids:

| Ex. | | |
|---|---|---|
| No. | Starting Amine | Data |
| 47 | 42 | R1 = propyl; R2 = F |
| | | LRMS: m/z ES 498 [MH]⁺ |
| 48 | 42 | R1 = methylcyclopropyl; R2 = F |
| | | LRMS: m/z ES 510 [MH]⁺ |
| 49 | 42 | R1 = 2,2-difluorocyclobutyl; R2 = F |
| | | LRMS: m/z ES 546 [MH]⁺ |
| 50 | 42 | R1 = 2-methylpropyl; R2 = F |
| | | LRMS: m/z ES 512 [MH]⁺ |
| 51 | 42 | R1 = 4,4-difluorocyclohexyl; R2 = F |
| | | LRMS: m/z ES 574 [MH]⁺ |
| 52 | 42 | R1 = tetrahydropyran-4-yl; R2 = F |
| | | LRMS: m/z ES 540 [MH]⁺ |
| 53 | 43 | R1 = 2,2-difluorocyclobutyl; R2 = H |
| | | LRMS: m/z ES 528 [MH]⁺ |
| 54 | 43 | R1 = 4,4-difluorocyclohexyl; R2 = H |
| | | LRMS: m/z ES 556 [MH]⁺ |
| 55 | 43 | R1 = methylcyclopropyl; R2 = H |
| | | LRMS: m/z ES 492 [MH]⁺ |
| 56 | 43 | R1 = propyl; R2 = H; LRMS: m/z ES 480 [MH]⁺ |

### EXAMPLES 57-63

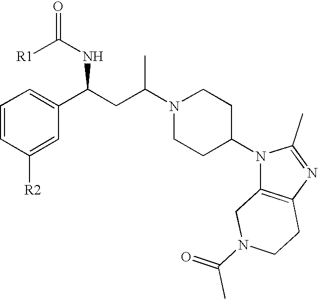

The procedure of Example 23 was followed reacting the compound of Example 42 or the compound of Example 43 with a set of acid chlorides:

| Ex. No. | Starting Amine | Data |
|---|---|---|
| 57 | 42 | R1 = cyclobutyl; R2 = F |
| | | LRMS: m/z ES 510 [MH]⁺ |
| 58 | 42 | R1 = isopropyl; R2 = F |
| | | LRMS: m/z ES 498 [MH]⁺ |
| 59 | 42 | R1 = methoxy; R2 = F |
| | | LRMS: m/z ES 486 [MH]⁺ |
| 60 | 42 | R1 = cyclopropyl; R2 = F |
| | | LRMS: m/z ES 496 [MH]⁺ |
| 61 | 42 | R1 = isopropoxy; R2 = F |
| | | LRMS: m/z ES 514 [MH]⁺ |
| 62 | 42 | R1 = ethyl; R2 = F |
| | | LRMS: m/z ES 484 [MH]⁺ |
| 63 | 43 | R1 = isopropoxy; R2 = H |
| | | LRMS: m/z ES 496 [MH]⁺ |

### EXAMPLES 64 AND 65

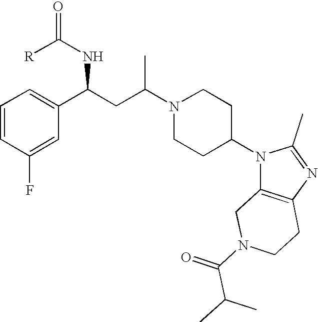

The procedure of Example 23 was followed reacting the compound of Example 44 with a set of acid chlorides:

| Ex. No. | Data |
|---|---|
| 64 | R = methyl; |
| | LRMS: m/z ES 498 [MH]⁺ |
| 65 | R = ethyl; |
| | LRMS: m/z ES 512 [MH]⁺ |

### EXAMPLE 66

### Ethyl 3-{1-[(3S)-3-(acetylamino)-3-(3-fluorophenyl)-1-methylpropyl]piperidin-4-yl}-2-methyl-3,4,6,7-tetrahydro-5H-imidazo[4,5-c]pyridine-5-carboxylate

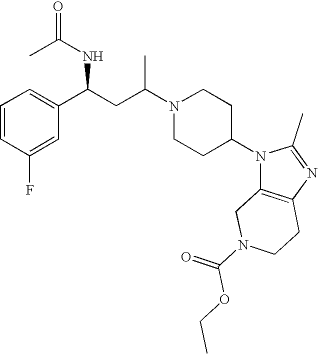

Acetyl chloride (0.0048 mL, 0.068 mmol) was added to a stirring solution of the compound of Example 45 (26 mg, 0.057 mmol) and triethylamine (0.0095 mL, 0.068 mmol) in dichloromethane (1.5 mL) and the mixture was stirred for 16 hours. Saturated sodium hydrogen carbonate solution (1 mL) was added to the mixture and the layers were separated. The aqueous layer was extracted with further dichloromethane (2×1 mL) and the combined organic fractions were dried (MgSO₄) and the solvent removed in vacuo. The residue was purified by column chromatography on silica gel using 95:5:0.5 dichloromethane:methanol:0.88 ammonia as eluant to afford the title compound as a white solid, 17.7 mg (63%).

LRMS: m/z ES 500 [MH]⁺

### EXAMPLES 67 AND 68

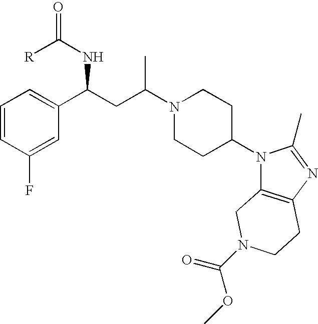

The procedure of Example 23 was followed reacting the compound of Example 46 with a set of acid chlorides:

| Ex. No. | Data |
|---|---|
| 67 | R = ethyl; |
| | LRMS: m/z ES 500 [MH]⁺ |
| 68 | R = isopropyl; |
| | LRMS: m/z ES 514 [MH]⁺ |

### EXAMPLE 69

### Methyl 3-{1-[(3S)-3-(butyrylamino)-3-(3-fluorophenyl)-1-methylpropyl]piperidin-4-yl}-2-methyl-3,4,6,7-tetrahydro-5H-imidazo[4,5-c]pyridine-5-carboxylate

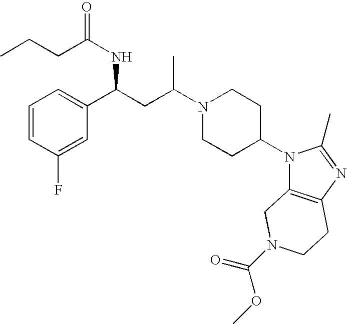

The procedure of Example 21 was followed reacting the compound of Example 46 with butyric acid to give the title compound as a white solid in 82% yield.

LRMS: m/z ES 514 [MH]⁺

### EXAMPLE 70

### Methyl 1-(1-{(3S)-3-[(tert-butoxycarbonyl)amino]-1-methyl-3-phenylpropyl}piperidin-4-yl)-2-methyl-1,4,6,7-tetrahydro-5H-imidazo[4,5-c]pyridine-5-carboxylate

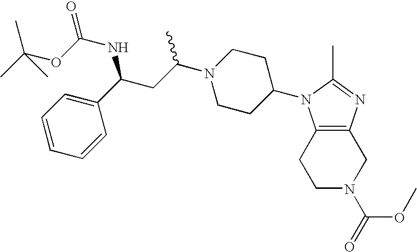

The free base of the compound of Preparation 15 (1 g, 3.6 mmol) and tert-butyl [(1S)-3-oxo-1-phenylbutyl]carbamate (473 mg, 1.8 mmol) (preparation 42) were dissolved in dichloromethane (20 mL) and titanium tetraisopropoxide (2.66 mL, 9.0 mmol) was added. The mixture was stirred at room temperature under nitrogen for 48 hours and then sodium cyanoborohydride (179 mg, 2.7 mmol) dissolved in methanol (2 mL) was added. After 4 hours, the mixture was poured onto a mixture of ice-cooled ethyl acetate (50 mL) and saturated sodium hydrogen carbonate (5 mL). The mixture was filtered through Arbocel®, the filtrate was washed with saturated sodium hydrogen carbonate (2×40 mL), dried (MgSO₄) and the solvent removed under reduced pressure. The residue was purified by column chromatography on silica gel using 98:2:0.2 dichloromethane:methanol:0.88 ammonia as eluant to give the title compound as a white solid, 847 mg (89%).

LRMS: m/z APCI 526 [MH]⁺

### EXAMPLE 71

### Methyl 1-{1-[(3S)-3-amino-1-methyl-3-phenylpropyl]piperidin-4-yl}-2-methyl-1,4,6,7-tetrahydro-5H-imidazo[4,5-c]pyridine-5-carboxylate

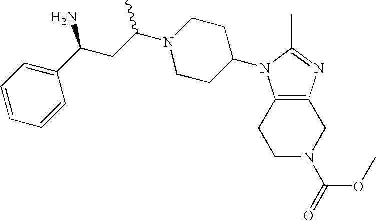

The procedure of Example 4 was followed reacting the compound of Example 70 with ethereal hydrogen chloride to give the title compound as the hydrochloride salt which was converted to the free base in 80% yield.

LRMS: m/z APCI 426 [MH]⁺

### EXAMPLES 72 AND 73

### Methyl 1-{1-[(3S)-3-(acetylamino)-1-methyl-3-phenylpropyl]piperidin-4-yl}-2-methyl-1,4,6,7-tetrahydro-5H-imidazo[4,5-c]pyridine-5-carboxylate

The compound of Example 71 (547 mg, 1.29 mmol) was dissolved in dichloromethane (10 mL) and triethylamine (0.27 mL, 1.94 mmol) was added. The mixture was cooled to 0° C. and put under nitrogen. Acetyl chloride (0.09 mL, 1.29 mmol) was added dropwise and the reaction allowed to warm to room temperature and stirred for 16 hours. Saturated sodium hydrogen carbonate (10 mL) was added and the mixture was extracted with dichloromethane (3×15 mL). The combined organic fractions were dried (MgSO₄) and the solvent removed in vacuo. The residue was purified by column chromatography on silica gel using an elution gradient of dichloromethane:methanol (98:2 to 95:5) to give 7 mg of the compound of Example 72 as a yellow solid (first eluting diastereoisomer), 280 mg of a mixture of diastereoisomers and 9 mg of the compound of Example 73 as a yellow solid (second eluting diastereoisomer):

For Example 72: ¹H-NMR (CD₃OD, 400 MHz): δ 7.36-7.29 (4H, m), 7.24 (1H, m), 5.08 (1H, m), 4.35 (2H, m), 3.97 (1H, m), 3.77-3.73 (2H, m), 3.71 (3H, m), 2.92-2.76 (4H, m), 2.68 (1H, m), 2.58 (1H, m), 2.37 (3H, s), 2.33 (1H, m), 2.21-2.00 (3H, m), 1.97 (3H, s), 1.87-1.78 (2H, m), 1.70 (1H, m), 1.04 (3H, d).

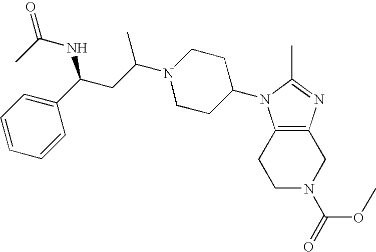

For Example 73: ¹H-NMR (CD₃OD, 400 MHz): δ 7.38-7.29 (4H, m), 7.24 (1H, m), 5.05 (1H, m), 4.36 (2H, m), 4.05 (1H, m), 3.80-3.69 (2H, m), 3.72 (3H, s), 3.07 (1H, m), 2.90-2.80 (3H, m), 2.75-2.60 (2H, m), 2.52 (1H, m), 2.39 (3H, s), 2.29-2.04 (3H, m), 1.96 (3H, s), 1.94-1.85 (2H, m), 1.73 (1H, m), 1.08 (3H, d).

LRMS: m/z APCI 468 [MH]⁺

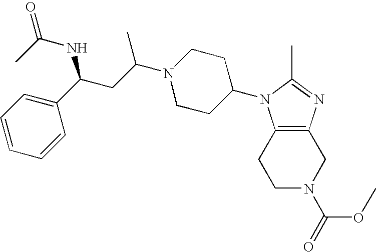

### EXAMPLE 74

### Methyl 1-{1-[(3S)-3-[(tert-butoxycarbonyl)amino]-3-(3-fluorophenyl)-1-methylpropyl]piperidin-4-yl}-2-methyl-1,4,6,7-tetrahydro-5H-imidazo[4,5-c]pyridine-5-carboxylate

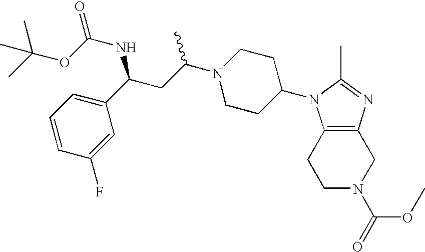

The procedure of Example 70 was followed reacting the compound of Preparation 15 with tert-butyl [(1S)-1-(3-fluorophenyl)-3-oxobutyl]carbamate (preparation 43) to give the title compound as an oil, in 63% yield.

LRMS: m/z ES 544 [MH]⁺

### EXAMPLE 75

### Methyl 1-{1-[(3S)-3-amino-3-(3-fluorophenyl)-1-methylpropyl]piperidin-4-yl}-2-methyl-1,4,6,7-tetrahydro-5H-imidazo[4,5-c]pyridine-5-carboxylate

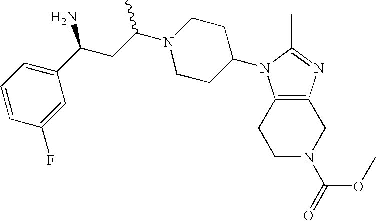

The procedure of Example 4 was followed reacting the compound of Example 74 with ethereal hydrogen chloride to give the title compound as the hydrochloride salt which was converted to the free base as a white solid in 80% yield.

LRMS: m/z ES 544 [MH]⁺

### EXAMPLES 76 TO 79

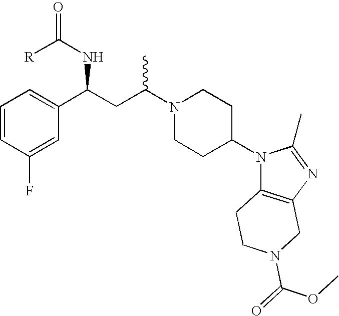

The appropriate acid chloride (1 eq) was added to a solution of the compound of Example 75 (1 eq.) and triethylamine (1.5 eq.) in dichloromethane (30 mLmmol⁻¹) at 0° C. under nitrogen. The reaction was allowed to warm to room temp and stirred for 18 hours. The reaction mixture was evaporated under reduced pressure, the residue partitioned between dichloromethane and 10% sodium bicarbonate solution and this mixture shaken for 30 minutes. The layers were separated, the aqueous solution extracted with dichloromethane and the combined organic extracts evaporated under reduced pressure to afford the title compound. The residue was purified by column chromatography on silica gel using dichloromethane:methanol as eluant to give the title compounds.

| Ex. No. | Data |
|---|---|
| 76 | R = methyl; |
| | LRMS: m/z ES 486 [MH]⁺ |
| 77 | R = isopropyl; |
| | LRMS: m/z ES 514 [MH]⁺ |
| 78 | R = cyclopropyl; |
| | LRMS: m/z ES 512 [MH]⁺ |
| 79 | R = cyclobutyl; |
| | LRMS: m/z ES 526 [MH]⁺ |

### EXAMPLE 80

### Methyl 1-{1-[(3S)-3-{[(3,3-difluorocyclobutyl)carbonyl]amino}-3-(3-fluorophenyl)-1-methylpropyl]piperidin-4-yl}-2-methyl-1,4,6,7-tetrahydro-5H-imidazo[4,5-c]pyridine-5-carboxylate

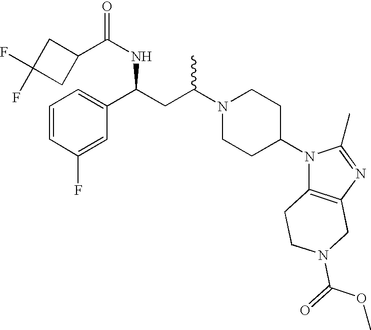

The compound of Example 75 (150 mg, 0.34 mmol) and 3,3-difluorocyclobutanecarboxylic acid (69 mg, 0.51 mmol) were dissolved in dichloromethane (10 mL) and 3-(diethoxyphosphoryloxy)-1,2,3-benzotriazin-4(3H)-one (153 mg, 0.51 mmol) was added followed by N,N-diisopropylethylamine (0.12 mL, 0.68 mmol). The mixture was stirred at room temperature under nitrogen for 16 hours. The reaction mixture was partitioned between dichloromethane (40 mL) and saturated sodium hydrogen carbonate solution (40 mL). The organic layer was dried (MgSO₄) and the solvent removed under reduced pressure. The residue was purified by column chromatography on silica gel using an elution gradient of dichloromethane to 95:5 dichloromethane:methanol as eluant to give the title compound as an oil, 160 mg (83%).

LRMS: m/z ES 562 [MH]⁺

### EXAMPLES 81 AND 82

### Methyl 1-(1-{(3S)-3-[(tert-butoxycarbonyl)amino]-1-methyl-3-phenylpropyl}piperidin-4-yl)-2-methyl-1,4,6,7-tetrahydro-5H-imidazo[4,5-c]pyridine-5-carboxylate

The compound of Example 74 (730 mg) was separated into its component diastereoisomers using a Gemini-Base column and diethylamine (0.05%) in water and diethylamine (0.05%) in acetonitrile as mobile phase. 273 mg of the compound of Example 81 (first eluting diastereoisomer) and 172 mg of the compound of Example 82 (second eluting diastereoisomer) were obtained as white solids.

For Example 81: LRMS: m/z ES 526 [MH]⁺; ¹H-NMR (CD₃OD, 400 MHz): δ 7.34-7.27 (4H, m), 7.21 (1H, m), 4.67 (1H, m), 4.37 (2H, m), 3.98 (1H, m), 3.79-3.73 (2H, m), 3.72 (3H, s), 2.98 (1H, m), 2.92-2.81 (2H, m), 2.76 (1H, m), 2.63 (1H, m), 2.52 (1H, m), 2.38 (3H, s), 2.36 (1H, m), 2.23-2.09 (2H, m), 1.97 (1H, m), 1.93-1.82 (2H, m), 1.59 (1H, m), 1.40 (9H, s), 1.02 (3H, d).

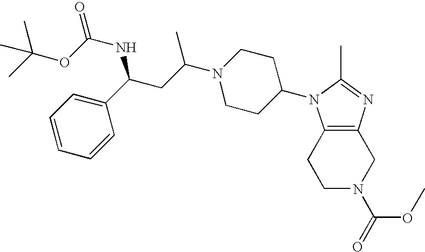

For Example 82: LRMS: m/z ES 526 [MH]⁺; ¹H-NMR (CD₃OD, 400 MHz): δ 7.34-7.26 (4H, m), 7.21 (1H, m), 4.80 (1H, m), 4.36 (2H, m), 3.96 (1H, m), 3.76-3.72 (2H, m), 3.71 (3H, s), 2.92 (1H, m), 2.87-2.81 (2H, m), 2.78 (1H, m), 2.68 (1H, m), 2.59 (1H, m), 2.38 (3H, s), 2.30 (1H, m), 2.24-1.93 (3H, m), 1.91-1.80 (2H, m), 1.63 (1H, m), 1.42 (9H, s), 0.98 (3H, d).

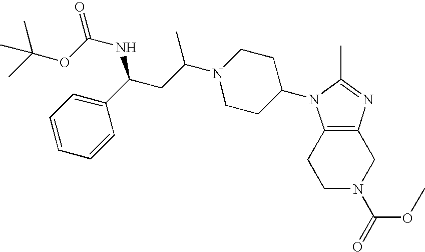

### EXAMPLE 83

### Methyl1-{1-[(3S)-3-amino-1-methyl-3-phenylpropyl]piperidin-4-yl}-2-methyl-1,4,6,7-tetrahydro-5H-imidazo[4,5-c]pyridine-5-carboxylate

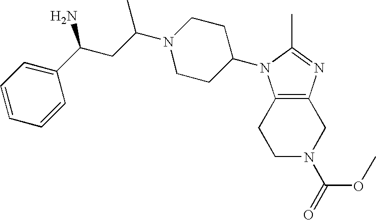

The procedure of Example 4 was followed reacting the compound of Example 81 with ethereal hydrogen chloride to give the title compound as the hydrochloride salt which was converted to the free-base as a white solid in 84% yield.

LRMS: m/z APCI 426 [MH]⁺

### EXAMPLE 84

### Methyl 1-{1-[(3S)-3-amino-1-methyl-3-phenylpropyl]piperidin-4-yl}-2-methyl-1,4,6,7-tetrahydro-5H-imidazo[4,5-c]pyridine-5-carboxylate

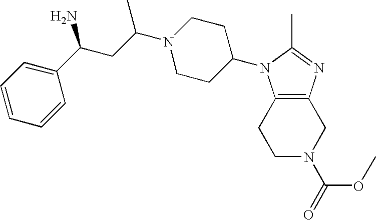

The procedure of Example 4 was followed reacting the compound of Example 82 with ethereal hydrogen chloride to give the title compound as the hydrochloride salt which was converted to the free-base as a white solid in 88% yield.

LRMS: m/z APCI 426 [MH]⁺

### EXAMPLE 85

### Ethyl 1-{1-[(3S)-3-[(tert-butoxycarbonyl)amino]-3-(3-fluorophenyl)-1-methylpropyl]piperidin-4-yl}-2-methyl-1,4,6,7-tetrahydro-5H-imidazo[4,5-c]pyridine-5-carboxylate

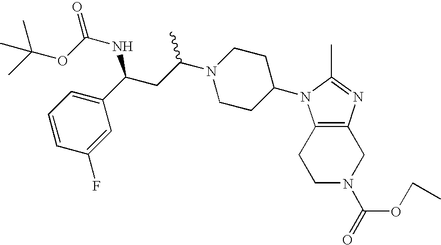

The procedure of Example 70 was followed reacting the compound of Preparation 18 with tert-butyl [(1S)-1-(3-fluorophenyl)-3-oxobutyl]carbamate (preparation 43) in the presence of titanium tetraisopropoxide and sodium cyanoborohydride to give the title compound as a white solid in 52% yield.

LRMS: m/z APCI 559 [MH]⁺

### EXAMPLE 86

### tert-Butyl [(1S)-3-[4-(5-acetyl-2-methyl-4,5,6,7-tetrahydro-1H-imidazo[4,5-c]pyridin-1-yl)piperidin-1-yl]-1-(3-fluorophenyl)butyl]carbamate

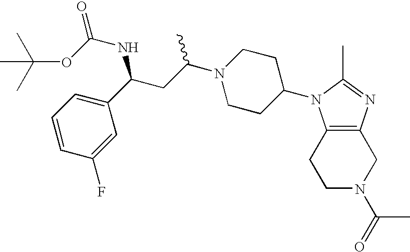

The procedure of Example 70 was followed reacting the compound of Preparation 19 with tert-butyl [(1S)-1-(3-fluorophenyl)-3-oxobutyl]carbamate (preparation 43) in the presence of titanium tetraisopropoxide and sodium cyanoborohydride to give the title compound as a white solid in 59% yield.

LRMS: m/z APCI 529 [MH]⁺

### EXAMPLES 87 AND 88

### Ethyl 1-{1-[(3S)-3-amino-3-(3-fluorophenyl)-1-methylpropyl]piperidin-4-yl}-2-methyl-1,4,6,7-tetrahydro-5H-imidazo[4,5-c]pyridine-5-carboxylate

The procedure of Example 4 was followed reacting the compound of Example 85 with ethereal hydrogen chloride to give the title compound as the hydrochloride salt which was converted to the free-base as a white solid in 72% yield.

LRMS: m/z ES 458 [MH]⁺

The compound was separated into its component diastereoisomers using a Chiralcel OD-H column with a mobile phase of 70:30:0.1 hexane/isopropanol/diethylamine to give the compound of Example 87 (first eluting diastereoisomer) as a white solid, 466 mg and the compound of Example 88 (second eluting diastereoisomer) as a white solid, 250 mg.

For Example 87: ¹H-NMR (CD₃OD, 400 MHz): δ 7.33 (1H, m), 7.18 (1H, d), 7.14 (1H, m), 6.96 (1H, m), 4.36 (2H, s), 4.15 (2H, q), 4.07 (1H, t), 3.96-3.87 (2H, m), 3.75 (2H, t), 3.01 (1H, m), 2.86-2.79 (2H, m), 2.74-2.52 (2H, m), 2.38 (3H, s), 2.32 (1H, m), 2.19-1.94 (3H, m), 1.92-1.81 (2H, m), 1.54 (1H, m), 1.26 (3H, t), 0.96 (3H, d).

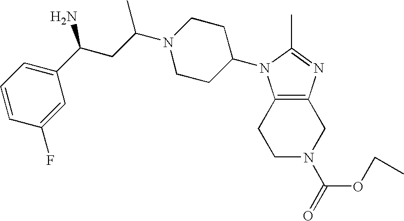

For Example 88: ¹H-NMR (CD₃OD, 400 MHz): δ 7.35 (1H, m), 7.17 (1H, d), 7.14 (1H, m), 6.98 (1H, m), 4.35 (2H, s), 4.15 (2H, q), 4.02 (1H, m), 3.99-3.86 (1H, m), 3.73 (2H, t), 2.88-2.74 (4H, m), 2.60 (1H, m), 2.51 (1H, m), 2.36 (3H, s), 2.30 (1H, m), 2.14-1.89 (3H, m), 1.86-1.77 (2H, m), 1.69 (1H, m), 1.26 (3H, t), 1.04 (3H, d).

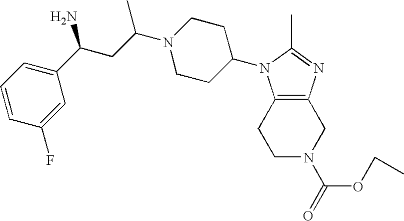

### EXAMPLES 89 AND 90

### (1S)-3-[4-(5-Acetyl-2-methyl-4,5,6,7-tetrahydro-1H-imidazo[4,5-c]pyridin-1-yl)piperidin-1-yl]-1-(3-fluorophenyl)butan-1-amine

The procedure of Example 4 was followed reacting the compound of Example 86 with ethereal hydrogen chloride to give the title compound as the hydrochloride salt which was converted to the free-base as a white solid in 69% yield.

LRMS: m/z ES 428 [MH]⁺

The compound was separated into its component diastereoisomers using a Chiralcel OD-H column with a mobile phase of 70:30:0.1 hexane/isopropanol/diethylamine to give the compound of Example 89 (first eluting diastereoisomer) as a white solid, 450 mg and the compound of Example 90 (second eluting diastereoisomer) as a white solid, 376 mg.

For Example 89: ¹H-NMR (CD₃OD, 400 MHz): δ 7.35 (1H, m), 7.19 (1H, d), 7.15 (1H, m), 6.98 (1H, m), 4.46+4.43 (1H, 2×d), 4.11 (1H, m), 3.99 (1H, m), 3.87 (1H, m), 3.79 (1H, m), 3.02 (1H, m), 2.91 (1H, m), 2.82 (1H, m), 2.76-2.53 (3H, m), 2.39+2.38 (3H, 2×s), 2.33 (1H, m), 2.18+2.14 (3H, 2×s), 2.17-1.95 (3H, m), 1.94-1.81 (2H, m), 1.57 (1H, m), 0.97 (3H, d).

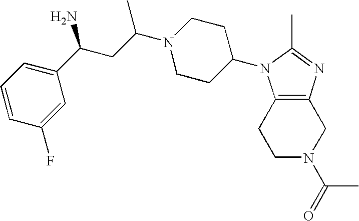

For Example 90: ¹H-NMR (CD₃OD, 400 MHz): δ 7.38 (1H, m), 7.19 (1H, d), 7.16 (1H, m), 7.01 (1H, m), 4.45+4.41 (2H, 2×s), 4.08 (1H, m), 4.00-3.89 (2H, m), 3.85 (1H, m), 3.76 (1H, m), 2.89-2.73 (4H, m), 2.62 (1H, m), 2.52 (1H, m), 2.37+2.36 (3H, 2×s), 2.30 (1H, m), 2.18+2.13 (3H, 2×s), 2.11-1.98 (2H, m), 1.94 (1H, m), 1.87-1.77 (2H, m), 1.72 (1H, m), 1.05+1.04 (3H, 2×d).

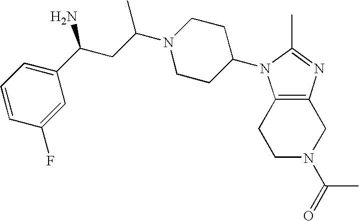

### EXAMPLES 91 TO 104

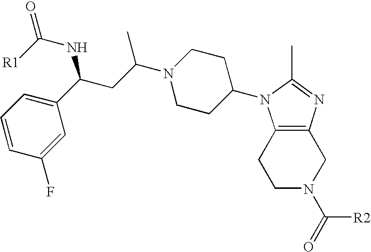

The procedure of Example 23 was followed reacting the amines below with a set of acid chlorides:

| Ex. No. | Starting Amine | Data |
|---|---|---|
| 91 | 83 | R1 = ethyl; R2 = methoxy |
| | | LRMS: m/z APCI 482 [MH]⁺ |
| 92 | 84 | R1 = ethyl; R2 = methoxy |
| | | LRMS: m/z APCI 482 [MH]⁺ |
| 93 | 89 | R1 = isopropyl; R2 = methyl |
| | | LRMS: m/z ES 498 [MH]⁺ |
| 94 | 90 | R1 = isopropyl; R2 = methyl |
| | | LRMS: m/z ES 498 [MH]⁺ |
| 95 | 87 | R1 = isopropyl; R2 = ethoxy |
| | | LRMS: m/z ES 528 [MH]⁺ |
| 96 | 88 | R1 = isopropyl; R2 = ethoxy |
| | | LRMS: m/z ES 528 [MH]⁺ |
| 97 | 87 | R1 = cyclopropyl; R2 = ethoxy |
| | | LRMS: m/z APCI 527 [MH]⁺ |
| 98 | 87 | R1 = ethyl; R2 = ethoxy |
| | | LRMS: m/z ES 515 [MH]⁺ |
| 99 | 88 | R1 = cyclopropyl; R2 = ethoxy |
| | | LRMS: m/z ES 527 [MH]⁺ |
| 100 | 88 | R1 = ethyl; R2 = ethoxy |
| | | LRMS: m/z ES 515 [MH]⁺ |
| 101 | 89 | R1 = cyclopropyl; R2 = methyl |
| | | LRMS: m/z ES 497 [MH]⁺ |
| 102 | 89 | R1 = ethyl; R2 = methyl |
| | | LRMS: m/z ES 485 [MH]⁺ |
| 103 | 90 | R1 = cyclopropyl; R2 = methyl |
| | | LRMS: m/z ES 497 [MH]⁺ |
| 104 | 90 | R1 = ethyl; R2 = methyl |
| | | LRMS: m/z ES [MH]⁺ |

### EXAMPLE 105

### N-{(1S)-3-[4-(3-isoPropyl-1,2,4-oxadiazol-5-yl)piperidin-1-yl]-1-phenylbutyl}cyclobutanecarboxamide

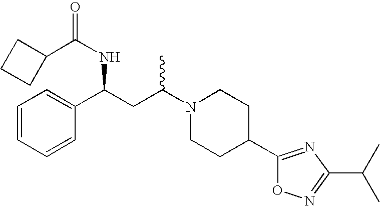

The procedure of Example 70 was followed reacting N-[(1S)-3-oxo-1-phenylbutyl]cyclobutanecarboxamide (preparation 44) with 4-(3-isopropyl-1,2,4-oxadiazol-5-yl)piperidine (preparation 33) in the presence of titanium tetraisopropoxide and sodium cyanoborohydride to give, after chromatography, the title compound as a white solid in 23% yield.

LRMS: m/z ES 425 [MH]⁺

### EXAMPLE 106A AND 106B

### tert-Butyl {(1S)-3-[4-(3-methyl-1,2,4-oxadiazol-5-yl)piperidin-1-yl]-1-phenylbutyl}carbamate

The procedure of example 70 was followed reacting tert-butyl [(1S)-3-oxo-1-phenylbutyl]carbamate (preparation 42) with 4-(3-methyl-1,2,4-oxadiazol-5-yl)piperidine (preparation 34) in the presence of titanium tetraisopropoxide and sodium cyanoborohydride. The residue was purified by column chromatography on silica gel using dichloromethane to 98:2:0.2 dichloromethane:methanol:0.88 ammonia as eluant to afford the first diastereoisomer, the compound of Example 106A, (1.5 g) as a yellow oil followed by some mixed fractions and then the second diastereoisomer, the compound of Example 106B (460 mg) as a yellow oil.

For Example 106A: LRMS: m/z APCI 415 [MH]⁺; ¹H-NMR (CD₃OD, 400 MHz): δ 7.33-7.18 (5H, m), 4.78 (1H, m), 3.01-2.83 (3H, m), 2.71 (1H, m), 2.68-2.52 (2H, m), 2.32 (3H, s), 2.29 (1H, m), 2.16-1.82 (5H, m), 1.60 (1H, m), 1.39 (9H, s), 0.94 (3H, d).

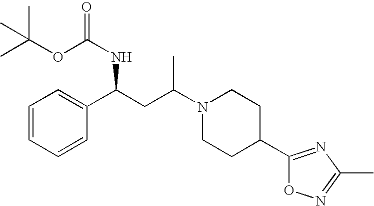

For Example 106B: LRMS: m/z APCI 415 [MH]⁺; ¹H-NMR (CD₃OD, 400 MHz): δ 7.32-7.17 (5H, m), 4.64 (1H, m), 3.00-2.88 (2H, m), 2.72 (1H, m), 2.65-2.46 (3H, m), 2.35 (1H, m), 2.33 (3H, s), 2.15-2.03 (2H, m), 1.99-1.82 (3H, m), 1.65 (1H, m), 1.39 (9H, s), 1.01 (3H, d).

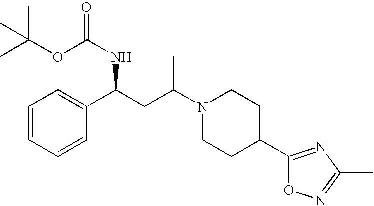

### EXAMPLE 107

### (1S)-3-[4-(3-Methyl-1,2,4-oxadiazol-5-yl)piperidin-1-yl]-1-phenylbutan-1-amine

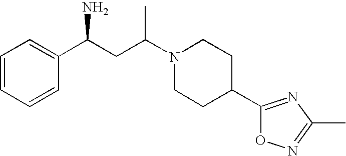

The procedure of Example 4 was followed reacting the compound of Example 106A with ethereal hydrogen chloride to give the title compound as the hydrochloride salt which was converted to the free-base, as a white solid in 40% yield.

LRMS: m/z APCI-315 [MH]⁺

### EXAMPLE 108

### (1S)-3-[4-(3-Methyl-1,2,4-oxadiazol-5-yl)piperidin-1-yl]-1-phenylbutan-1-amine

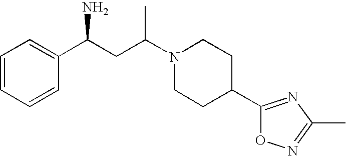

The procedure of Example 4 was followed reacting the compound of Example 106B with ethereal hydrogen chloride to give the title compound as the hydrochloride salt which was converted to the free-base as a white solid in 70% yield.

LRMS: m/z APCI 315 [MH]⁺

### EXAMPLE 109

### 4,4-Difluoro-N-{(1S)-3-[4-(3-methyl-1,2,4-oxadiazol-5-yl)piperidin-1-yl]-1-phenylbutyl}cyclohexanecarboxamide

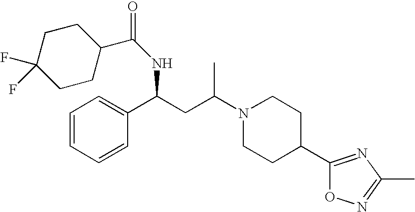

The procedure of Example 21 was followed reacting the compound of Example 107 with 4,4-Difluorocyclohexylcarboxylic acid to give the title compound as a colourless oil in 37% yield.

LRMS: m/z ES 461 [MH]⁺

### EXAMPLE 110

### 4,4-Difluoro-N-{(1S)-3-[4-(3-methyl-1,2,4-oxadiazol-5-yl)piperidin-1-yl]-1-phenylbutyl}cyclohexanecarboxamide

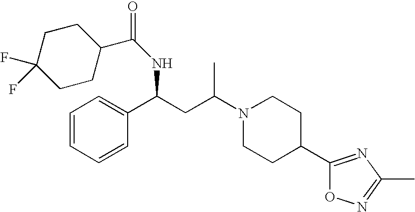

The procedure of Example 21 was followed reacting the compound of Example 108 with 4,4-Difluorocyclohexylcarboxylic acid to give the title compound as a colourless oil, 105 mg (71%).

LRMS: m/z ES 461 [MH]⁺

### EXAMPLE 111

### 4,4-Difluoro-N-{(1S)-3-[4-(2-isopropyl-1H-imidazol-1-yl)piperidin-1-yl]-1-phenylbutyl}cyclohexanecarboxamide

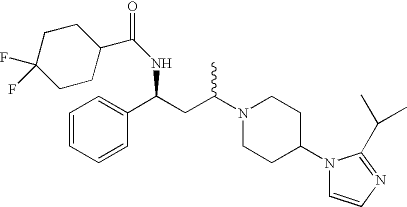

The procedure of Example 70 was followed reacting the compound of Example 136 with the compound of preparation 20 in the presence of titanium tetraisopropoxide and sodium cyanoborohydride to give the title compound as a white solid, 49 mg (72%).

LRMS: m/z APCI 487 [MH]⁺

### EXAMPLE 112

### 4,4-Difluoro-N-{(1S)-3-[4-(2-methyl-1H-imidazol-1-yl)piperidin-1-yl]-1-phenylbutyl}cyclohexanecarboxamide

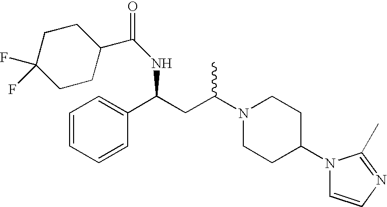

The procedure of Example 70 was followed reacting the compound of Example 136 with compound of preparation 21 in the presence of titanium tetraisopropoxide and sodium cyanoborohydride to give the title compound as a white solid in 61% yield.

LRMS: m/z APCI 459 [MH]⁺

### EXAMPLES 113A AND 113B

### tert-Butyl {(1S)-3-[4-(5-isopropyl-3-methyl-1H-pyrazol-1-yl)piperidin-1-yl]-1-phenylbutyl}carbamate

The procedure of Example 70 was followed reacting tert-butyl [(1S)-3-oxo-1-phenylbutyl]carbamate (preparation 42) with the compound of Preparation 24 in the presence of titanium tetraisopropoxide and sodium cyanoborohydride. The residue was purified by column chromatography on silica gel using an elution gradient of dichloromethane to 98:2:0.2 dichloromethane:methanol:0.88 ammonia as eluant to afford the first diastereoisomer, the compound of Example 113A (0.63 g) as a white solid followed by some mixed fractions and then the second diastereoisomer, the compound of Example 113B (0.30 g) as a white solid.

For Example 113A: LRMS: m/z APCI-455 [MH]⁺; ¹H-NMR (CD₃OD, 400 MHz): δ 7.34-7.27 (4H, m), 7.21 (1H, m), 5.81 (1H, m), 4.78 (1H, m), 3.98 (1H, m), 3.04-2.86 (2H, m), 2.77 (1H, m), 2.65-2.61 (2H, m), 2.36-2.16 (3H, m), 2.15 (3H, s), 2.02 (1H, m), 1.84-1.76 (2H, m), 1.63 (1H, ddd), 1.42 (9H, s), 1.22 (6H, dd), 0.97 (3H, d).

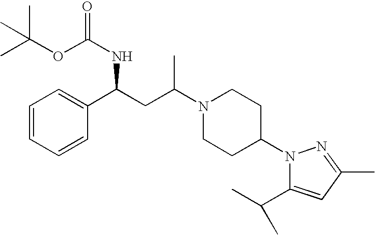

For Example 113B: LRMS: m/z APCI 455 [MH]⁺; ¹H-NMR (CD₃OD, 400 MHz): δ 7.36-7.27 (4H, m), 7.21 (1H, m), 5.82 (1H, m), 4.66 (1H, m), 3.99 (1H, m), 3.03-2.93 (2H, m), 2.74 (1H, m), 2.64-2.45 (2H, m), 2.40-2.20 (3H, m), 2.17 (3H, s), 1.98 (1H, m), 1.86-1.73 (2H, m), 1.57 (1H, m), 1.39 (9H, s), 1.22 (6H, dd), 1.01 (3H, s).

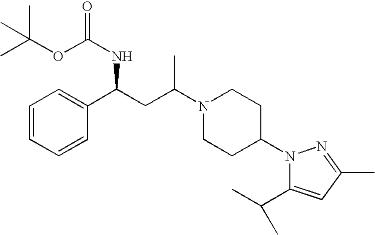

### EXAMPLE 114

### (1S)-3-[4-(5-isoPropyl-3-methyl-1H-pyrazol-1-yl)piperidin-1-yl]-1-phenylbutan-1-amine

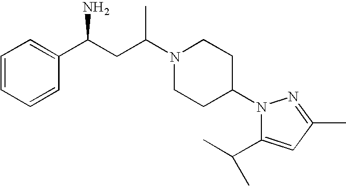

The procedure of Example 4 was followed reacting the compound of Example 113A with ethereal hydrogen chloride to give the title compound, the hydrochloride salt, as a white solid, 640 mg.

LRMS: m/z APCI 355 [MH]⁺

### EXAMPLE 115

### (1S)-3-[4-(5-isoPropyl-3-methyl-1H-pyrazol-1-yl)piperidin-1-yl]-1-phenylbutan-1-amine

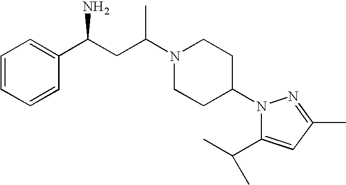

The procedure of Example 4 was followed reacting the compound of Example 113B with ethereal hydrogen chloride to give the title compound, the hydrochloride salt, as a white solid, 330 mg.

LRMS: m/z APCI 355 [MH]⁺

### EXAMPLE 116

### 4,4-Difluoro-N-{(1S)-3-[4-(5-isopropyl-3-methyl-1H-pyrazol-1-yl)piperidin-1-yl]-1-phenylbutyl}cyclohexanecarboxamide

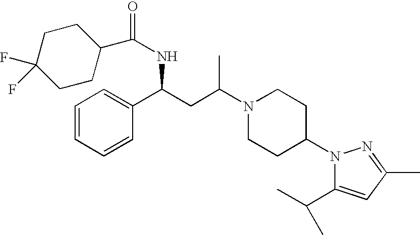

The procedure of Example 21 was followed reacting the compound of Example 114 with 4,4-Difluorocyclohexylcarboxylic acid in the presence of triethylamine to give the title compound as a white solid, 268 mg (%).

LRMS: m/z APCI 502 [MH]⁺

### EXAMPLE 117

### 4,4-Difluoro-N-{(1S)-3-[4-(5-isopropyl-3-methyl-1H-pyrazol-1-yl)piperidin-1-yl]-1-phenylbutyl}cyclohexanecarboxamide

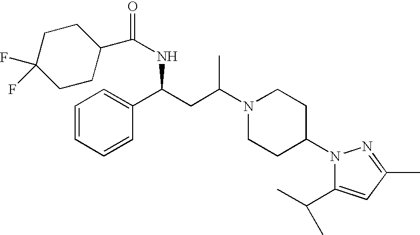

The procedure of Example 21 was followed reacting the compound of Example 115 with 4,4-Difluorocyclohexylcarboxylic acid in the presence of triethylamine to give the title compound as a white solid, 193 mg (%).

LRMS: m/z APCI 502 [MH]⁺

### EXAMPLES 118A AND 118B

### tert-Butyl {(1S)-3-[4-(5-isopropyl-1H-tetrazol-1-yl)piperidin-1-yl]-1-phenylbutyl}carbamate

The procedure of Example 70 was followed reacting tert-Butyl [(1S)-3-oxo-1-phenylbutyl]carbamate (preparation 42) with the compound of Preparation 27 in the presence of titanium tetraisopropoxide and sodium cyanoborohydride. The residue was purified by column chromatography on silica gel using 99.5:0.5:0.05 to 99:1:0.1 dichloromethane:methanol:0.88 ammonia as eluant to afford the first diastereoisomer, the compound of Example 118A (93 mg) as a white solid followed by some mixed fractions and then the second diastereoisomer, the compound of Example 118B (61 mg) as a white solid.

For Example 118A: LRMS: m/z ES 443 [MH]⁺; ¹H-NMR (CD₃OD, 400 MHz): δ 7.35-7.27 (4H, m), 7.21 (1H, m), 4.83 (1H, m), 4.40 (1H, m), 3.34 (1H, m), 3.00 (1H, m), 2.82 (1H, m), 2.68 (2H, m), 2.43-2.22 (3H, m), 2.09-1.99 (3H, m), 1.64 (1H, m), 1.43 (9H, s), 1.38 (6H, d), 0.98 (3H, d).

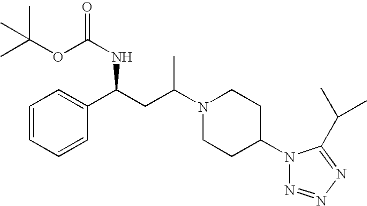

For Example 118B: LRMS: m/z ES 443 [MH]⁺; ¹H-NMR (CD₃OD, 400 MHz): δ 7.36-7.27 (4H, m), 7.22 (1H, m), 4.68 (1H, m), 4.41 (1H, m), 3.36 (1H, m), 3.06-2.74 (2H, m), 2.71-2.21 (4H, m), 2.19-1.89 (4H, m), 1.59 (1H, m), 1.43-1.34 (15H, m), 1.03 (3H, d).

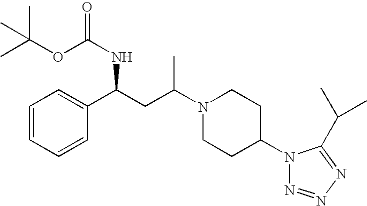

### EXAMPLE 119

### (1S)-3-[4-(5-isoPropyl-1H-tetrazol-1-yl)piperidin-1-yl]-1-phenylbutan-1-amine

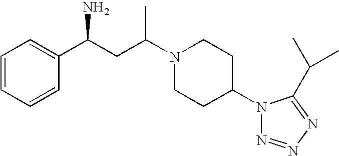

The procedure of Example 19 was followed reacting the compound of Example 118A with hydrogen chloride gas to give the title compound as the hydrochloride salt which was converted to the free-base as a colourless oil in 89% yield.

LRMS: m/z ES 343 [MH]⁺

### EXAMPLE 120

### (1S)-3-[4-(5-isoPropyl-1H-tetrazol-1-yl)piperidin-1-yl]-1-phenylbutan-1-amine

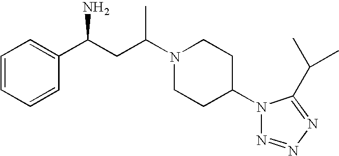

The procedure of Example 19 was followed reacting the compound of Example 118B with hydrogen chloride gas to give the title compound as the hydrochloride salt which was converted to the free-base as a colourless oil in 80% yield.

LRMS: m/z ES 343 [MH]⁺

### EXAMPLE 121

### 4,4-Difluoro-N-{(1S)-3-[4-(5-isopropyl-1H-tetrazol-1-yl)piperidin-1-yl]-1-phenylbutyl}cyclohexanecarboxamide

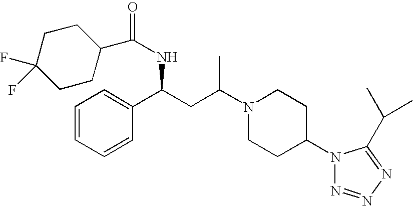

The procedure of Example 21 was followed reacting the compound of Example 119 with 4,4-Difluorocyclohexylcarboxylic acid to give the title compound as a white solid in quantitative yield.

LRMS: m/z ES 489 [MH]⁺

### EXAMPLE 122

### 4,4-Difluoro-N-{(1S)-3-[4-(5-isopropyl-1H-tetrazol-1-yl)piperidin-1-yl]-1-phenylbutyl}cyclohexanecarboxamide

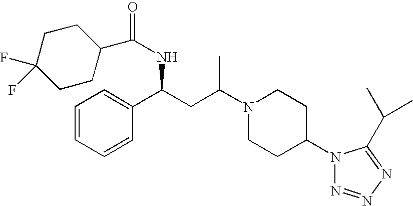

The procedure of Example 21 was followed reacting the compound of Example 120 with 4,4-Difluorocyclohexylcarboxylic acid to give the title compound as a white solid in 80% yield.

LRMS: m/z ES 489 [MH]⁺

### EXAMPLE 123

### tert-Butyl {(1S)-3-[4-(1-isopropyl-1H-tetrazol-5-yl)piperidin-1-yl]-1-phenylbutyl}carbamate

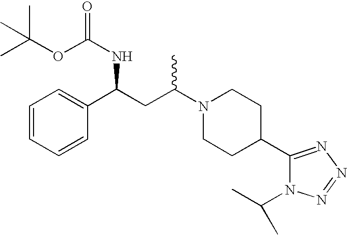

The procedure of Example 70 was followed reacting tert-Butyl [(1S)-3-oxo-1-phenylbutyl]carbamate (preparation 42) with the compound of Preparation 31 in the presence of titanium tetraisopropoxide and sodium cyanoborohydride. The residue was purified by column chromatography on silica gel using 98:2:0.2 dichloromethane:methanol:0.88 ammonia as eluant to give the title compound as a white solid in 35% yield.

LRMS: m/z ES 443 [MH]⁺

### EXAMPLE 124

### (1S)-3-[4-(1-isopropyl-1H-tetrazol-5-yl)piperidin-1-yl]-1-phenylbutan-1-amine

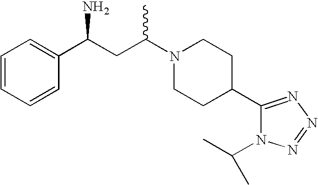

The procedure of Example 19 was followed reacting the compound of Example 123 with hydrogen chloride gas to give the title compound as the hydrochloride salt which was converted to the free-base, as a colourless oil in 75% yield.

LRMS: m/z ES 343 [MH]⁺

### EXAMPLES 125 AND 126

### 4,4-difluoro-N-{(1S)-3-[4-(1-isopropyl-1H-tetrazol-5-yl)piperidin-1-yl]-1-phenylbutyl}cyclohexanecarboxamide

The procedure of Example 21 was followed reacting the compound of Example 119 with 4,4-Difluorocyclohexylcarboxylic acid. The residue was purified by column chromatography on silica gel using 99:1:0.1 to 97.5:2.5:0.25 dichloromethane:methanol:0.88 ammonia as eluant to afford the first diastereoisomer, the compound of Example 125 as a white solid, 26 mg followed by some mixed fractions and then the second diastereoisomer, the compound of Example 126, as a white solid, 14 mg.

For Example 125: LRMS: m/z APCI 489 [MH]⁺

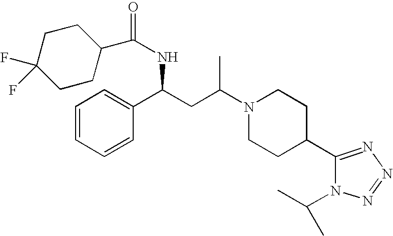

For Example 126: LRMS: m/z APCI 489 [MH]⁺

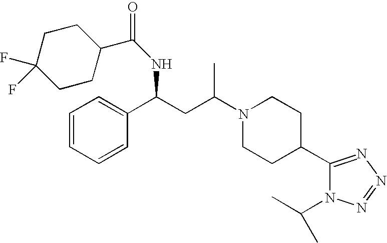

### EXAMPLES 127 TO 131

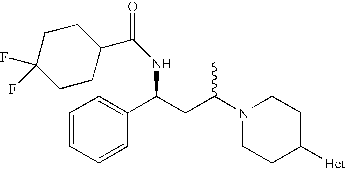

The procedure of Example 70 was followed reacting the compound of Example 136 with a set of 4 substituted piperidine heterocycles in the presence of titanium tetraisopropoxide and sodium cyanoborohydride to give the title compounds as detailed below.

| Ex. No. | Het | Data |
|---|---|---|
| 127 | 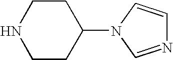 | LRMS: m/z APCl [MH]⁺ |
| | (prep. 32) | |
| 128 | 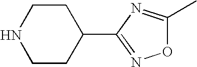 | LRMS: m/z APCl [MH]⁺ |
| | (prep. 34) | |
| 129 | 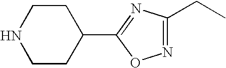 | LRMS: m/z APCl [MH]⁺ |
| | (prep. 35) | |
| 130 | 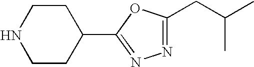 | LRMS: m/z APCl [MH]⁺ |
| | (prep. 36) | |
| 131 | 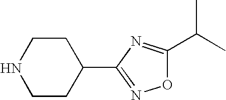 | LRMS: m/z APCl [MH]⁺ |
| | (prep. 35) | |

### EXAMPLE 132

### tert-butyl {(1S)-3-[4-(5-acetyl-2-methyl-4,5,6,7-tetrahydro-3H-imidazo[4,5-c]pyridin-3-yl)piperidin-1-yl]-1-phenylbutyl}carbamate

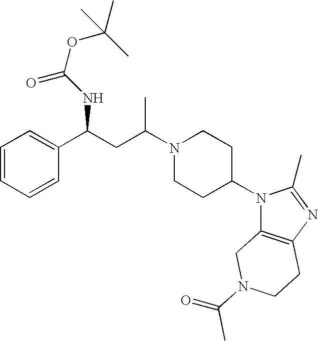

The procedure of Example 36 was followed reacting the compound of Example 15 with acetyl chloride and potassium carbonate in isopropyl alcohol to give the title compound in 67% yield.

LRMS: m/z ES 510 [MH]⁺

### EXAMPLE 133

### tert-butyl {(1S)-3-[4-(5-acetyl-2-methyl-4,5,6,7-tetrahydro-3H-imidazo[4,5-c]pyridin-3-yl)piperidin-1-yl]-1-phenylbutyl}carbamate

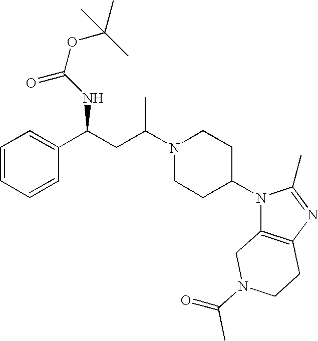

The procedure of Example 36 was followed reacting the compound of Example 16 with acetyl chloride and potassium carbonate in isopropyl alcohol to give the title compound in 68% yield.

LRMS: m/z ES 510 [MH]⁺

### EXAMPLE 134

### (1S)-3-[4-(5-acetyl-2-methyl-4,5,6,7-tetrahydro-3H-imidazo[4,5-c]pyridin-3-yl)piperidin-1-yl]-1-phenylbutan-1-amine

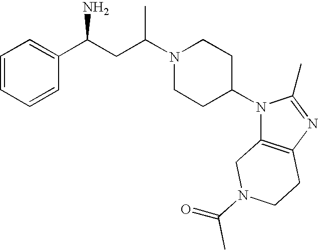

The procedure of Example 19 was followed reacting the compound of Example 132 with hydrogen chloride gas to give the title compound, which was converted to the free base, as a white solid in 85% yield.

LRMS: m/z ES 410 [MH]⁺

### EXAMPLE 135

### (4S)-4-amino-4-phenylbutan-2-one

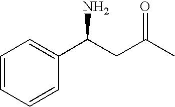

tert-Butyl [(1S)-3-oxo-1-phenylbutyl]carbamate (3.0 g, 11.4 mmol) (preparation 42) was dissolved in dichloromethane (50 mL) and 2N ethereal hydrochloric acid (50 mL) was added. The mixture was stirred at room temperature for 4 hours and then the solvent was removed in vacuo to give the title compound as a yellow solid, 2.5 g.

LRMS: m/z APCI 164 [MH]⁺

### EXAMPLE 136

### 4,4-difluoro-N-[(1S)-3-oxo-1-phenylbutyl]cyclohexanecarboxamide

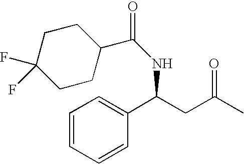

The procedure of Example 21 was followed reacting the compound of Example 135 with 4,4-Difluorocyclohexylcarboxylic acid to give the title compound as a white solid in 70% yield.

LRMS: m/z APCI-310 [MH]⁺

### Biological Data

The ability of the compounds of formula (I) and their pharmaceutically acceptable salts, solvates and derivatives to modulate chemokine receptor activity is demonstrated by methodology known in the art, such as by using the assay for CCR5 binding following procedures disclosed in Combadiere et al., J. Leukoc. Biol., 60, 147-52 (1996); and/or by using the intracellular calcium mobilisation assays as described by the same authors, and/or inhibiting cell fusion following procedures disclosed in Bradley et al., J Biomol Screen 9, 516-24 (2004).

Cell lines expressing the receptor of interest include those naturally expressing the receptor, such as PM-1, or IL-2 stimulated peripheral blood lymphocytes (PBL), or a cell engineered to express a recombinant receptor, such as CHO, 300.19, L1.2 or HEK-293.

The pharmacological activity of the compounds of formula (I) and their pharmaceutically acceptable salts, solvates and derivatives is further demonstrated using a gp160 induced cell-cell fusion assay to determine the IC₅₀ values of compounds against HIV-1 fusion. The gp160 induced cell-cell fusion assay uses a HeLa P4 cell line and a CHO-Tat10 cell line.

The HeLa P4 cell line expresses CCR5 and CD4 and has been transfected with HIV-1 LTR-β-Galactosidase. The media for this cell line is Dulbecco modified eagle's medium (D-MEM) (without L-glutamine) containing 10% foetal calf serum (FCS), 2 mM L-glutamine, penicillin/streptomycin (Pen/Strep; 100 U/mL penicillin+10 mg/mL streptomycin), and 1 μg/ml puromycin.

The CHO cell line is a Tat (transcriptional trans activator)-expressing clone from a CHO JRR17.1 cell line that has been transfected with pTat puro plasmid. The media for this cell line is rich medium for mammalian cell culture originally developed at Roswell Park Memorial Institute RPMI1640 (without L-glutamine) containing 10% FCS, 2 mM L-glutamine, 0.5 mg/ml Hygromycin B and 12 pg/ml puromycin. The CHO JRR17.1 line expresses gp160 (JRFL) and is a clone that has been selected for its ability to fuse with a CCR5/CD4 expressing cell line.

Upon cell fusion, Tat present in the CHO cell is able to transactivate the HIV-1 long terminal repeat (LTR) present in the HeLa cell leading to the expression of the β-Galactosidase enzyme. This expression is then measured using a Fluor Ace™ β-Galactosidase reporter assay kit (Bio-Rad cat no. 170-3150). This kit is a quantitative fluorescent assay that determines the level of expression of β-galactosidase using 4-methylumbelliferyl-galactopyranoside (MUG) as substrate. β-Galactosidase hydrolyses the fluorogenic substrate resulting in release of the fluorescent molecule 4-methylumbelliferone (4MU). Fluorescence of 4-methylumbelliferone is then measured on a fluorometer using an excitation wavelength of 360 nm and emission wavelength of 460 nm.

Compounds that inhibit fusion will give rise to a reduced signal and, following solubilisation in an appropriate solvent and dilution in culture medium, a dose-response curve for each compound can be used to calculate IC₅₀ values.

The compounds of formula (I) have an IC₅₀ in the above cell fusion assay, of less than 1.5 μM

| | Example No | | | | | | | | | | | |
|---|---|---|---|---|---|---|---|---|---|---|---|---|
| | 2 | 3 | 5 | 6 | 7 | 8 | 9A | 9B | 10 | 11 | 12 | 13A |
| IC₅₀ | 14.0 | 434 | 0.7 | 1.0 | 0.9 | 6.0 | 2.5 | 5.9 | 0.3 | 0.6 | 0.6 | 37.1 |
| (nM) | | | | | | | | | | | | |
| | Example No | | | | | | | | | | | |
| | 13B | 14 | 21 | 22 | 23 | 24 | 25 | 26 | 27 | 28 | 29 | 30 |
| C₅₀ | 600 | 17.2 | 0.1 | 0.5 | 0.1 | 0.2 | 0.4 | 38.5 | 115 | 79.2 | 29.9 | 11.0 |
| (nM) | | | | | | | | | | | | |
| | Example No | | | | | | | | | | | |
| | 31 | 47 | 48 | 49 | 50 | 51 | 52 | 53 | 54 | 55 | 56 | 57 |
| C₅₀ | 191 | 0.7 | 0.8 | 0.4 | 0.4 | 0.3 | 1.0 | 0.5 | 0.4 | 0.3 | 1.2 | 0.2 |
| (nM) | | | | | | | | | | | | |
| | Example No | | | | | | | | | | | |
| | 58 | 59 | 60 | 61 | 62 | 63 | 64 | 65 | 66 | 67 | 68 | 69 |
| C₅₀ | 0.5 | 13.0 | 0.6 | 1.6 | 1.0 | 1.8 | 2.0 | 0.6 | 3.5 | 0.2 | 0.1 | 0.2 |
| (nM) | | | | | | | | | | | | |
| | Example No | | | | | | | | | | | |
| | 72 | 73 | 76 | 77 | 78 | 79 | 80 | 91 | 92 | 93 | 94 | 95 |
| C₅₀ | 2.4 | 9.1 | 5.5 | 0.6 | 1.8 | 0.1 | 0.2 | 102 | 0.4 | 12.9 | 0.2 | 5.5 |
| (nM) | | | | | | | | | | | | |
| | Example No | | | | | | | | | | | |
| | 96 | 97 | 98 | 99 | 100 | 101 | 102 | 103 | 104 | 105 | 106A | 109 |
| C₅₀ | 0.1 | 10.0 | 19.1 | 0.1 | 0.1 | 18.8 | 25.9 | 0.2 | 0.8 | 94.6 | 362 | 4.6 |
| (nM) | | | | | | | | | | | | |

| | Example No | | | | | | | | | | | | |
|---|---|---|---|---|---|---|---|---|---|---|---|---|---|
| | 110 | 111 | 112 | 116 | 117 | 121 | 125 | 126 | 127 | 128 | 129 | 130 | 131 |
| C₅₀ | 353 | 0.2 | 1.0 | 50.5 | 1,170 | 0.5 | 2.2 | 21.8 | 4.7 | 26.0 | 8.6 | 5.9 | 20.2 |
| (nM) | | | | | | | | | | | | | |

In general, the compounds tested displayed acceptable metabolic stability in our liver microsome in vitro assay.

1. A compound of formula (I): 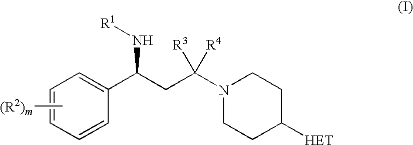
or a pharmaceutically acceptable salt, solvate or derivative thereof, wherein:
R¹ is COR⁵; CO₂R⁵; or CONR⁶R⁷;
R² is halogen, cyano, CF₃, C₁₋₄alkyl, or C₁₋₄alkyloxy;
R³ is C₁₋₄ alkyl;
R⁴ is H or C₁₋₄alkyl;
R⁵ is C₁₋₆alkyl; C₃₋₇cycloalkyl; or C₃₋₇cycloalkyl-C₁₋₂alkyl, wherein said alkyl and cycloalkyl are substituted by 0 to 3 halogen atoms; or a 4 to 7-membered saturated heterocycle containing one O or one S atom and wherein when the S atom is present, it is substituted by 0 to 2 oxo groups;
R⁶ is C₁₋₆alkyl;
R⁷ is H or C₁₋₆alkyl;
m is 0, 1 or 2;
n is 1 or 2;
HET is a:
(i) a 5 membered monocylic aromatic heterocycle containing from 1 to 4 heteroatoms selected from the group consisting of O, S and N, which is optionally substituted by C₁₋₄ alkyl, C₃₋₆cycloalkyl, C₁₋₄alkyloxy and C₁₋₄alkyloxyC₁₋₄alkyl; or
(ii) a tetrahyrodroimadazopyridine of formula 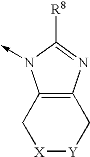
wherein:
R⁸ is methyl or ethyl substituted by 0 to 3 fluorine atoms;
X and Y are selected from CH₂ and NR⁹ such that one of X and Y is CH₂ and the other is NR⁹;
R⁹ is COR⁶; CO₂R⁶; or CONR⁶R⁷
with the proviso:
(i) that when R¹ is CO₂R⁵, R⁵ is not a tertiary alkyl group; and
(ii) that HET is not a 1,2,4-triazole or a 1,3,4-triazole.

2. The compound as claimed in claim 1 wherein the monocylic aromatic Het is selected from the following moieties: 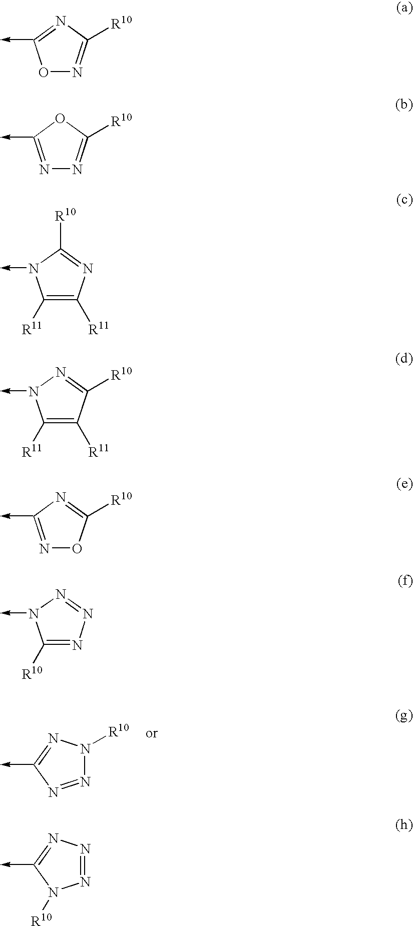
wherein R¹⁰ and each R¹¹ are independently selected from H, C₁₋₄ alkyl, C₃₋₆cycloalkyl, C₁₋₄alkyloxy and C₁₋₄alkyloxyC₁₋₄alkyl; or a pharmaceutically acceptable salt, solvate or derivative thereof.

3. The compound as claimed in claim 1 wherein R¹⁰ is C₁₋₄ alkyl, C₃₋₆cycloalkyl, C₁₋₄alkyloxy or C₁₋₄alkyloxy-C₁₋₄alkyl; and R¹¹ is H; or a pharmaceutically acceptable salt, solvate or derivative thereof.

4. The compound as claimed in claim 1 wherein R¹⁰ is C₁₋₄ alkyl or a pharmaceutically acceptable salt, solvate or derivative thereof.

5. The compound as claimed in claim 1 wherein HET is a tetrahydroimadazopyridine to give a formula (IA) 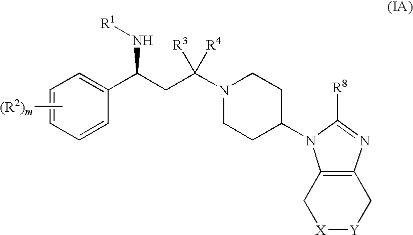
or a pharmaceutically acceptable salt, solvate or derivative thereof,
wherein R¹ to R⁴ are as defined in claim 1, R⁸ is methyl or ethyl substituted by 0 to 3 fluorine atoms;
one of X and Y are selected from CH₂ and NR⁹ such that one of X and Y are CH₂ and the other is NR⁹; and
R⁹ is COR⁶, CO₂R⁶ or CONR⁶R⁷, wherein R⁶ and R⁷ are as defined in claim 1.

6. The compound as claimed in claim 1 wherein R⁸ is methyl; or a pharmaceutically acceptable salt, solvate or derivative thereof.

7. The compound as claimed in claim 1 wherein R⁹ is COR⁶ or CO₂R⁶; or a pharmaceutically acceptable salt, solvate or derivative thereof.

8. The compound as claimed in claim 1 wherein R⁶ is methyl, ethyl, n-propyl or isopropyl; or a pharmaceutically acceptable salt, solvate or derivative thereof.

9. The A compound as claimed in claim 1 wherein the saturated heterocycle of R⁵ is 1,1-dioxo-tetrahydrothiopyran or tetrahydropyran; or a pharmaceutically acceptable salt, solvate or derivative thereof.

10. The compound as claimed in claim 1 wherein R¹ is COR⁵ or CO₂R⁵; or a pharmaceutically acceptable salt, solvate or derivative thereof.

11. The compound as claimed in claim 1 wherein R¹ is COR⁵ or CO₂R⁵ and R⁵ is C₁₋₄alkyl or C₃₋₇cycloalkyl wherein the cycloalkyl is optionally substituted by 0 to 2 fluoro atoms; or a pharmaceutically acceptable salt, solvate or derivative thereof.

12. The compound as claimed in claim 1 wherein R² is halogen; or a pharmaceutically acceptable salt, solvate or derivative thereof.

13. The compound as claimed in claim 1 wherein m is 0 or 1; or a pharmaceutically acceptable salt, solvate or derivative thereof.

14. The compound as claimed in claim 1 wherein R³ is methyl; or a pharmaceutically acceptable salt, solvate or derivative thereof.

15. The compound as claimed in claim 1 wherein R⁴ is H; or a pharmaceutically acceptable salt, solvate or derivative thereof.

16. The compound as claimed in claim 5 wherein R¹ is COR⁵ or CO₂R⁵ wherein R⁵ is C₁₋₄alkyl; m is 0 or 1 and R² is halogen; R³ is methyl; R⁴ is H; R⁸ is methyl; and R⁹ is COR⁶ or CO₂R⁶ wherein R⁶ is C₁₋₄alkyl; or a pharmaceutically acceptable salt, solvate or derivative thereof.

17. A compound selected from the group consisting of
N-{(1S)-3-[4-(5-acetyl-2-methyl-4,5,6,7-tetrahydro-3H-imidazo[4,5-c]pyridin-3-yl)piperidin-1-yl]-1-phenylbutyl}butanamide;
N-[(1S)-3-[4-(5-acetyl-2-methyl-4,5,6,7-tetrahydro-3H-imidazo[4,5-c]pyridin-3-yl)piperidin-1-yl]-1-(3-fluorophenyl)butyl]-2-methylpropanamide;
N-[(1S)-3-[4-(5-acetyl-2-methyl-4,5,6,7-tetrahydro-3H-imidazo[4,5-c]pyridin-3-yl)piperidin-1-yl]-1-(3-fluorophenyl)butyl]propanamide;
ethyl 3-{1-[(3S)-3-(acetylamino)-3-(3-fluorophenyl)-1-methylpropyl]piperidin-4-yl}-2-methyl-3,4,6,7-tetrahydro-5H-imidazo[4,5-c]pyridine-5-carboxylate;
methyl 2-methyl-1-{1-[(3S)-1-methyl-3-phenyl-3-(propionylamino)propyl]piperidin-4-yl}-1,4,6,7-tetrahydro-5H-imidazo[4,5-c]pyridine-5-carboxylate; N-[(1S)-3-[4-(5-acetyl-2-methyl-4,5,6,7-tetrahydro-1H-imidazo[4,5-c]pyridin-1-yl)piperidin-1-yl]-1-(3-fluorophenyl)butyl]-2-methylpropanamide; and
N-[(1S)-3-[4-(5-acetyl-2-methyl-4,5,6,7-tetrahydro-1H-imidazo[4,5-c]pyridin-1-yl)piperidin-1-yl]-1-(3-fluorophenyl)butyl]propanamide;
or a pharmaceutically acceptable salt, solvate or derivatives thereof.

18. A pharmaceutical composition comprising a compound of formula (I) or a pharmaceutically acceptable salt, solvate or derivative thereof as claimed in claim 1 together with one or more pharmaceutically acceptable excipients, diluents or carriers.

19. The pharmaceutical composition as claimed in claim 18 comprising one or more additional therapeutic agents.

20. (canceled)

21. (canceled)

22. (canceled)

23. (canceled)

24. (canceled)

25. A method of treating a disorder in which the modulation of CCR5 receptors is implicated which comprises administering to a patient in need thereof a therapeutically effective amount of a compound of formula (I) or a pharmaceutically acceptable salt, solvate or derivative thereof as claimed in claims 1.

26. The method according to claim 25 wherein the disorder is HIV, a retroviral infection genetically related to HIV, AIDS, an inflammatory disease, an autoimmune disease, or pain.

27. A process for preparing a compound of formula (I) as defined in claim 1 or a pharmaceutically acceptable salt, solvate or derivative thereof which comprises:
(a) reacting a compound of formula V: 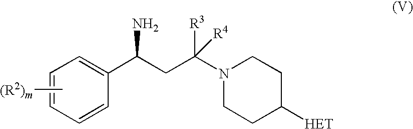
with R¹X, wherein X is a leaving group; or
(b) reacting a compound of formula (VI) with a compound of formula (IV) 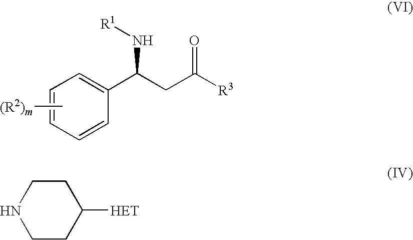
and optionally converting the compound obtained in step (a) or step (b) to a pharmaceutically acceptable salt, solvate or derivative thereof;
wherein R¹, R², R³, R⁴, m and HET are as defined in claim 1

28. A process for preparing a compound of formula (V) which comprises deprotecting a compound of formula (II) 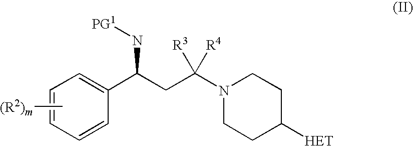
wherein R², R³, R⁴, m and HET are as defined in claim 1 and PG¹ is a nitrogen protecting group.

29. A compound of formula 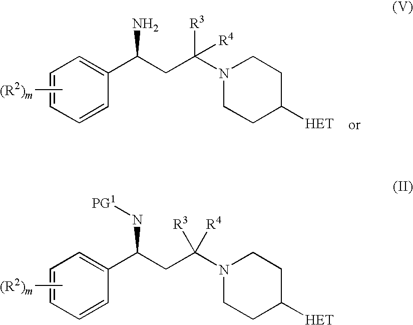
wherein R², R³, R⁴, m and HET are as defined in claim 1 and PG¹ is a nitrogen protecting group.
